(19) United States
(12) Patent Application Publication
(10) Pub. No.: US 2025/0262268 A1
(43) Pub. Date: Aug. 21, 2025

(54) Methods and Compositions for Inhibiting Viral Infection

(71) Applicants: New York Genome Center, Inc., New York, NY (US); New York University, New York, NY (US)

(72) Inventors: Neville E. Sanjana, New York, NY (US); Zharko Daniloski, New York, NY (US)

(21) Appl. No.: 18/041,627

(22) PCT Filed: Aug. 18, 2021
(86) PCT No.: PCT/US2021/046512

§ 371 Date: Jun. 2, 2023

Related U.S. Application Data
(60) Provisional application No. 63/092,827, filed on Oct. 16, 2020.
(60) Provisional application No. 63/066,950, filed on Aug. 18, 2020.

(51) Int. Cl. A61K 38/06; A61K 31/138; A61K 31/405; A61K 31/4985; A61K 31/506; A61K 31/5377; A61K 31/706; A61P 31/14; C12N 9/22; C12N 15/10; C12N 15/11; C12N 15/86; C12Q 1/6883

(52) U.S. Cl. CPC A61K 38/06; A61K 31/138; A61K 31/405; A61K 31/4985; A61K 31/506; A61K 31/5377; A61K 31/706; A61P 31/14; C12N 9/226; C12N 15/1082; C12N 15/111; C12N 15/86; C12Q 1/6883; C12N 2310/20; C12N 2740/15043; C12Q 2600/136; C12Q 2600/156

(57) ABSTRACT

A method of treating or inhibiting a viral infection in a human subject, such as a SARS-CoV-2 infection, involves inhibiting in vivo the expression or activity of one or a combination of the subject's genes required for viral infection. Single genes or subsets of genes for inhibition of activity or expression are selected from certain identified genes. Methods of administration of certain known small molecules or other therapeutics which mimic loss of function of the identified genes are provided. Similar methods for conducting screens of host genes required for viral infection are shown.

### CROSS-REFERENCE TO RELATED APPLICATIONS

This application claims the benefit of the priorities of U.S. Provisional Patent Application No. 63/066,950 filed Aug. 18, 2020, and U.S. Provisional Patent Application No. 63/092,827, filed Oct. 16, 2020, which applications are incorporated herein by reference.

### STATEMENT REGARDING FEDERALLY SPONSORED RESEARCH OR DEVELOPMENT

This invention was made with government support under grant number R01AI123155, HR0011-20-2-0040, R01MH106842, DP2HG010099, UM1HG008901, R01HL142028, R01CA218668, HR0011-20-2-0040 and D18AP00053 awarded by the National Institutes of Health. The government has certain rights in this invention.

### INCORPORATION-BY REFERENCE OF MATERIAL SUBMITTED IN ELECTRONIC FORM

Applicant hereby incorporates by reference the Sequence Listing file labeled “NYGLIPP-118_US_ST25” (created on Jun. 2, 2023, 2,401 bytes).

### BACKGROUND OF THE INVENTION

As of August 2020, SARS-CoV-2 (Severe Acute Respiratory Syndrome-Coronavirus-2), the virus that causes COVID-19, has infected more than 19 million people worldwide and led to the deaths of more than 700,000 people, according to the John Hopkins Research Center (Gardner, 2020). SARS-CoV-2 belongs to the family enveloped viruses known as Coronaviridae and was first reported in late 2019 in China. Over the past two decades, it is the third zoonotic virus to emerge: Compared to the other two coronaviruses, SARS-CoV (2002) and Middle East Respiratory Syndrome (MERS)-CoV (2012), SARS-CoV-2 shows an increased infectivity and lower case-fatality rate, contributing to its wide-spread transmission and resulting in a pandemic (Gates, 2020; Liu et al., 2020). Given that SARS-CoV-2 has already taken a major toll on human life and livelihoods worldwide, many research institutions, governmental organizations and pharmaceutical companies are working to identify antiviral drugs and develop new vaccines. Currently, there are at least 28 vaccines against SARS-CoV-2 in clinical trials and one FDA approved antiviral drug (remdesivir) that acts as an inhibitor of the SARS-CoV-2 viral RNA-dependent RNA polymerase (Beigel et al., 2020; Funk et al., 2020). A recent study identified small molecules that antagonize SARS-CoV-2 replication and infection by testing ˜12,000 clinical-stage and FDA-approved inhibitors (Riva et al., 2020).

SARS-CoV-2 is an enveloped positive-sense RNA virus that relies on host factors for all stages of its lifecycle (Kim et al., 2020; Zhou et al., 2020). The viral envelope is coated by Spike protein heterotrimers that bind to angiotensin converting-enzyme 2 (ACE2) receptor, which is required for SARS-CoV-2 infection (Hoffmann et al., 2020a; Zhou et al., 2020). The Spike protein undergoes proteolytic cleavage that is catalyzed several host proteases, such as furin, TMPRSS2 and Cathepsin L, and can occur in the secretory pathway of the host cell or during viral entry in the target cell. Proteolytic cleavage is considered to be required for activation of Spike that in turn allows for viral-host membrane fusion and release of the viral RNA into the host cytoplasm (Hoffmann et al., 2020b). Once in the cytoplasm, the virus utilizes the host and its own machinery to replicate its genetic material and assemble new viral particles. Recent proteomic studies have identified hundreds of host genes that directly bind to SARS-CoV-2 viral proteins and have mapped changes in the global protein phosphorylation landscape in response to viral infection, highlighting the interest in better understanding of host-virus genetic dependencies (Bouhaddou et al., 2020; Gordon et al., 2020).

A continuing need in the art exists for new and effective tools and methods for the treatment and prevention of viruses, including SARs-CoV-2.

### SUMMARY OF THE INVENTION

To better understand host-virus genetic dependencies and find potential therapeutic targets, we performed a genome-scale CRISPR loss-of-function screen in human alveolar basal epithelial cells to identify genes whose loss confers resistance to SARS-CoV-2 viral infection. In the case of SARS-CoV-2, we identify genes whose loss confers resistance to viral infection of human alveolar basal epithelial cells. These gene targets (and inhibitors of these genes) provide new therapies in the battle against COVID-19.

We validate that these genes reduce SARS-CoV-2 infection using multiple orthogonal cell perturbations (CRISPR knock-out, RNA interference knock-down, and small-molecule inhibitors). For the top gene hits, we explore mechanisms of their antiviral activity using single-cell transcriptomics, flow cytometry and immunofluorescence. In one embodiment, we show that loss of RAB7A reduces viral entry by sequestering ACE2 receptors inside cells through altered endosomal trafficking. Top-ranked genes cluster into distinct pathways, including the vacuolar ATPase proton pump, Retromer, the Commander complex, and the Arp2/3 complex, with multiple genes in each pathway highly enriched in the CRISPR screen.

We demonstrate that genes required for SARS-CoV-2 infection in vitro also contribute to COVID-19 severity in human patients and that we can use the pooled CRISPR screen to pinpoint likely protein-coding genes responsible for noncoding variants associated with COVID-19 risk in human patients.

In one aspect, a method of treating or inhibiting a viral infection in a human subject comprises inhibiting in vivo the expression or activity of one or a combination of the subject's genes required for viral infection, wherein said gene or combination is selected from the genes identified in Tables I, II, III or IV. In one embodiment, such inhibition is temporary for the duration of the viral infection, if established, or to suppress susceptibility to viral infection for a short time period when the virus is present in the community.

In another embodiment, a method of treating, preventing or inhibiting a viral infection in a human subject comprises administering to a mammalian subject in need thereof one or more inhibitors of the activity or expression of said genes or combination of genes. In one embodiment, such inhibition is temporary for the duration of the viral infection, if established, or to suppress susceptibility to viral infection for a short time period when the virus is present in the community. Such inhibitors may be selected from one or a combination of the inhibitors identified in Tables III or IV. In other embodiment, the inhibitor is administered to the subject pre-infection or post-infection at a dosage effective to mimic a loss of function of its corresponding gene.

In one aspect, a method of treating or inhibiting a viral infection in a human subject comprises inhibiting in vivo the expression or activity of one or a combination of the subject's genes required for SARS-CoV-2 viral infection.

In one aspect, a method for identifying host target genes required for viral infection targeting lung cells comprising performing a genome-scale loss of function screen as described in the specification.

In another aspect, in concert with expression quantitative trait loci (eQTLs) and genome-wide association studies (GWAS), the pooled CRISPR screen can pinpoint protein-coding genes responsible for noncoding variants associated with COVID-19 risk in human patients and through which noncoding variants associated with COVID-19 risk in human patients may function. This method provides a quantitative resource of the impact of the loss of each host gene on response to viral infection for every protein-coding gene in the human genome.

Still other aspects and advantages of these compositions and methods are described further in the following detailed description of the preferred embodiments thereof.

### BRIEF DESCRIPTION OF THE DRAWINGS

FIG. 1A-1D. A genome-scale CRISPR loss-of-function screen to identify genes that prevent SARS-CoV-2 infection of human alveolar epithelial cells. FIG. 1A is an overview of the genome-scale loss-of-function screen for host factors in human A549^{ACE2} cells requires for SARS-CoV-2 infection. Screen was performed by transducing human lung A549-ACE2 cells with CRISPR sequences from a GeCKOv2 genome-wide CRISPR Knockout library used to identify human host genes required for SARS-CoV-2 infection. Six different CRISPR sequences were used/gene to determine if knockout of individual genes produced a consistent result. After 10 days of cell growth, the cells were selected with puromycin and infected with SARS-CoV-2 at a low multiplicity of infection (MOI). Surviving cells were examined for guide RNAs vs. cells which were not infected (Control) by counting sgRNA counts. FIG. 1B shows the percent survival of human A549^{ACE2} cells transduced with the GeCKOv2 library with the indicated SARS-CoV-2 viral amount (MOI) at 6 days post-infection. FIG. 1C is a scatterplot of guide RNA read counts from A549^{ACE2} cells at 6 days post-infection with SARS-CoV-2 (MOI˜0.01) versus cells prior to infection. Read counts are normalized log 2 reads. FIG. 1D is a volcano plot of median fold-change of guide RNAs for each gene and log 10 Robust Rank Aggregation (RRA) p-values. All genes with |fold-change|>4 and RRA p<10−3 are labeled. 54% of the top 50 ranked genes overlapped between the MOI 0.01 and MOI 0.3 screen.

FIG. 2 is a schematic of SARS-CoV-2 docking, entry, RNA genome release and transcription, and virion replication, assembly and release with top-ranked host genes from the CRISPR screen highlighted in red. All genes shown are ranked in the top 50 genes (top ˜0.25% of library) in the low MOI CRISPR screen using Robust Rank Aggregation.

FIGS. 3A-3E demonstrate that enriched genes cluster into related pathways, are expressed broadly, interact directly with viral proteins, and are also involved in viral pathogenesis of pandemic flu and ZIKA. FIG. 3A is a classification of genes shown in FIG. 2 (top-ranked ˜0.25% of the GeCKOv2 library) into specific complexes. FIG. 3B is a gene set enrichment analysis normalized enrichment scores for all significant (FDR q<0.1) Gene Ontology (GO) biological processes. FIG. 3C is the expression of top-ranked genes (same as in FIG. 3A) across the indicated human tissues from GTEx v8. Gene expression color scale is transcripts per million (TPM). FIG. 3D shows the RRA fold-change for the low MOI CRISPR screen for the high-confidence protein-protein interaction with the maximum fold-change for each viral gene from the Gordon et al. mass spectrometry dataset (2020). FIG. 3E is a clustering of top-ranked GO biological processes for CRISPR screens for ZIKA (Y. Li et al., 2019), H1N1 pandemic avian influenza IAV (B. Li et al., 2020), and SARS-CoV-2 (this study).

FIGS. 4A-4E showarrayed validation of genome-scale SARS-CoV-2 screen and identification of druggable gene targets. FIG. 4A is a quantification of SARS-CoV-2 infected A549^{ACE2} knockout lines immuno-stained with N protein as shown in A. Each gene was targeted with 3 different guide RNAs represented as diamond symbols (n=3 biological replicates, error bars indicate s.e.m.). The graph showing the SARS-Cov-2 CRISPR KO Screen arrayed validation, plotting % virus infected cells resulting from knocking out (inhibiting or suppressing expression of) the human genes expressed by lung cells. The genes identified at the left hand side of the graph showed the greatest impact on infectivity when suppressed. (NT) refers to no perturbation/control. FIG. 4B shows the correlation of log 2 median fold change from the genome-scale CRISPR screen (low MOI) and percent of infected cells after individual (arrayed) gene perturbation shown in FIG. 4A. FIG. 4C shows druggable genes found in the Drug Gene Interaction database (DGIdb) among top 500 genes the genome-scale CRISPR screen ranked by RRA p-value. FIG. 4D shows the quantitative PCR (qPCR) of SARS-CoV-2 viral load present in A549^{ACE2} cells pretreated with 10 μM for 2 hours of indicated small molecule inhibitors and then infected with SARS-CoV-2 at MOI of 0.1. The qPCR was performed on cells collected at 36 hours post-infection (hpi). Red bars indicate inhibitors that yield a greater than 100-fold reduction in viral load. Bars with hatch marks indicate an unreliable viral load measurement due to a large reduction in cell viability (see FIG. 4E). Inhibitors were maintained at the same concentration throughout the experiment (n=6 biological replicates, error bars indicate s.e.m.). FIG. 4E is a percent of A549^{ACE2} viable cells following inhibitor treatments at 10 μM for 36 hpi determined using LIVE/DEAD stain and flow cytometry. Bars with red hatch marks indicate that inhibitor treatment had a large impact on viability (<90% viability). Significance testing for panels B, E, and F (data not shown).

FIG. 5A is a schematic of pooled CRISPR perturbations with Expanded CRISPR-compatible Cellular Indexing of Transcriptomes and Epitopes by sequencing (ECCITE-seq). FIG. 5B is a UMAP visualization of non-targeting and gene-perturbed cells after Linear Discriminant Analysis (LDA), revealing perturbation-specific clustering. FIG. 5C is a single-cell mRNA expression heatmap showing all differentially upregulated genes (adjusted p-value <0.01) for each target gene perturbation. Labeled genes were either a top-ranked gene from the genome-wide CRISPR screens (red) or among the top 5 differentially-expressed genes in any gene perturbation (black). FIG. 5D is a heatmap summarizing Gene Set Enrichment Analysis results for genes upregulated in any of the indicated target gene KO cells (all genes with p-value <0.01 and with a limit to the 300 most differentially-expressed genes; all enriched pathways with adjusted p-value <10⁻¹³).

FIG. 6A-6F demonstrate RAB7A loss results in a reduced cell surface expression and an increased endosomal accumulation of ACE2. FIG. 6A shows representative histograms of flow cytometry analysis to determine cell surface expression of ACE2 on A549 cell lines (A549 wild type (WT), A549^{ACE2}, and ACE2 with Cas9 and non-targeting (NT) or RAB7A-targeting guide RNAs). The dashed line indicates the gate between the ACE2 negative and positive cells. FIG. 6B is a quantification of cell surface expression of ACE2 across indicated A549 cell lines. ACE2 expression level was normalized across all samples to the A549^{ACE2} cells targeted with non-targeting (NT) guides (n=2-3 gRNA-transduced lines per gene, error bars are s.e.m.). FIG. 6C shows the percent of cells with ACE2 accumulation in vesicles in NT and RAB7A-transduced A549^{ACE2} cells (n=2 biological replicates, error bars are s.e.m.). FIG. 6D shows representative histograms of flow cytometry analysis to determine cell surface expression in Calu-3 cells. The dashed line indicates the gate between the ACE2 negative and positive cells. FIG. 6E shows a fraction of ACE2+ cells (using gating shown in 6D). ACE2 expression level was normalized across all samples to the Calu-3 cells transduced with a non-targeting (NT) guide (n=3 biological replicates, error bars are s.e.m.). FIG. 6F shows the mean area of ACE2 foci in Caco-2 cells transduced with a NT or a RAB7A-targeting guide (n=4 biological replicates, 80-105 cells per replicate were scored, error bars are s.e.m.). Significance testing for 6B and 6E was performed with a one-way ANOVA (B: F=9.8, p <10−4; G: Calu-3: F=378, p<10−4, Caco-2: F=222, p<10−4) with false-discovery rate-corrected post-hoc tests. Significance testing for 6B and 6C and 6F was performed with an unpaired t-test. For all panels, * indicates p≤0.05, ** indicates p≤0.01, and *** indicates p≤0.001, and **** indicates p≤0.0001.

FIGS. 7A-7D demonstrate genome-wide loss-of-function CRISPR screen enriched gene identification. FIG. 7A is a scatterplot of guide RNA read counts from A549^{ACE2} cells at 3 days post-transduction with the GeCKOv2 library versus read counts from the library plasmid. Read counts are normalized log 2 reads. FIG. 7B is a RRA p-value distribution for all genes in the GeCKOv2 library. FIG. 7C is the overlap of top-ranked (top 500) genes between 3 different analysis methods (RRA, RIGER, and SBR). 142 genes are found by all 3 methods. FIG. 7D is the overlap of top 50 ranked genes between the MOI 0.01 and MOI 0.3 screen.

FIGS. 8A-8E show gene set enrichment and overlap of top-ranked genes with other viral infections. FIG. 8A-8D show four of the significant (FDR<0.1) top-ranked GO biological process terms and the fold-change ranks of their genes in the SARS-CoV-2 low MOI CRISPR screen. FIG. 8E shows normalized gene ranks of the top 50 genes from the SARS-CoV-2 low MOI CRISPR screen in CRISPR screens for ZIKA and H1N1 avian influenza (IAV).

FIGS. 9A-9E show orthogonal perturbations of enriched CRISPR screen genes with RNAi and small-molecule inhibitors. FIG. 9A shows immunofluorescence quantification of SARS-CoV-2 N protein at 36 hours post-infection (hpi) at MOI 0.1 in A549^{ACE2} cells pretreated with siRNAs for 48 hours (n=3 technical replicates, error bars represent s.e.m., NT indicates non-targeting control guide RNA). FIG. 9B shows the immunofluorescence quantification of SARSCoV-2 N protein at 36 hpi (MOI 0.1) in A549^{ACE2} cells pretreated for 2 hours with 10 μM of the indicated inhibitors (n=3 biological replicates, error bars represent s.e.m.) FIG. 9C shows the immunofluorescence quantification of SARS-CoV-2 N protein at 36 hpi (MOI 0.1) in A549^{ACE2} cells pretreated with a combination of indicated inhibitors at 10 μM each for 2 hours (n=3 biological replicates, error bars represent s.e.m.). FIGS. 10D and 10E show CD8+ T-cells, preincubated for 1 hour with the indicated inhibitor followed by CD3/CD28 stimulation, stained for TNF-α or INFγ, respectively (n=3 biological replicates, error bars represent s.e.m.).

FIG. 10 shows the quantitative PCR (qPCR) of SARS-CoV-2 viral load present in A549ACE2 cells (CRISPR-perturbed with either non-targeting or PIK3C3-targeting guide RNAs) pretreated for 2 hours with the indicated PIK3C3 molecule inhibitors at 10 μM and then infected with SARS-CoV-2 at MOI of 0.1. The qPCR was performed at 36 hours post-infection (hpi). Inhibitors were maintained at the same concentration throughout the experiment (n=6 biological replicates, error bars indicate s.e.m.).

FIGS. 11A-11E show ECCITE-seq identifies common target gene signatures. FIG. 11A is a stacked violin plot of all genes targeted in ECCITE-seq (n=29; TMPRSS2 was not among the detected features). Single-cells are grouped by unique guide RNA target gene label of cells with a single detected guide RNA (n=4013 cells, median n=152 cells per target gene). Target gene expression is highlighted. FIG. 11B is a heatmap of Gene Set Enrichment Analysis results for genes downregulated in any of the indicated target gene perturbed cells (all genes with p-value <0.01 and with a limit to the 300 most differentially genes; all enriched pathways with adjusted p-value <0.01). FIG. 11C is a UMAP visualization of all DMSO and drug treated cells (n=3070 cells, median of n=604 cells per treatment) after running Linear Discriminant Analysis (LDA). Only enzastaurin clearly clusters away from DMSO control cells. FIGS. 11D-11E are heatmaps of Gene Set Enrichment Analysis results for genes upregulated (11D) and downregulated (11E) in any of the indicated treated cells to DMSO control cells (all genes with p-value <0.01 and with a limit to the 300 most differentially expressed genes; all enriched pathways with adjusted p-value <0.01).

FIGS. 12A-12B show flow cytometry for cell surface ACE2 expression and protein analysis of RAB7A protein after CRISPR targeting. FIGS. 12A and 12B show flow cytometry gating strategy to quantify cell surface expression of ACE2. FIG. 12A shows Live cells were first gated by the forward and side scatter area, then doublets were excluded by gating with the forward scatter area and width. Viable cells were selected by gating on side scatter area and LIVE/DEAD violet. FIG. 12B shows the gating strategy to determine ACE2+ cells. The gate was position such that <3% of A549 wild type and >85% of A549^{ACE2} cells were ACE2 positive. The same gating strategy was applied to all samples. Western blot on A549^{ACE2} cells perturbed with non-targeting (NT) or RAB7A-targeting guide RNAs and probed with a RAB7A antibody with GAPDH was used as loading control was performed (data not shown).

FIGS. 13A-13B demonstrate inflation of COVID-19 GWAS signal for eQTLs for CRISPR screen hit genes in lung and lead GWAS variant as an eQTL for the 8 genes in the 3p21.31 locus. FIGS. 13A and 13B are heatmaps showing the eQTL P-value of the lead variant 3:45867022:C:G from COVID-19 vs population GWAS (ANA_C2_V2) for the 8 genes in the 3p21.31 locus in 61 cell types and tissues from the eQTL Catalogue (13A) and 49 tissues from GTEx v8 (13B). A cell is colored in white, if there was no data for the variant-gene pair in the given eQTL dataset (e.g., the gene was not tested in eQTL mapping). eQTL datasets are clustered using the Euclidean distance and complete linkage method.

### DETAILED DESCRIPTION

Given the current COVID-19 global pandemic, there is an urgent need to better understand the complex relationships between host and virus genetic dependencies. We report a genome-wide loss-of-function screen in human lung cells that identified host genes required for SARS-CoV-2 viral infection. We selected and validated 30 genes that were ranked among the top 200 genes. To support the ability of the screen to identify key dependencies, some of the well-known host genes involved in SARS-CoV-2 Spike protein binding and entry such as the ACE2 receptor and Cathepsin L were among the top-scoring genes (Hoffmann et al., 2020a). One of the validated genes (SIGMAR) encodes the Sigma-1 receptor that was recently identified to be modulated by drugs effective against SARS-CoV-2 in vitro (Gordon et al., 2020). Overall, the top-ranked genes clustered within several protein complexes including vacuolar ATPases, Retromer and endosome, Commander, ARP2/3, PI3K and others, highlighting both the critical importance of multiple genes within each pathway to viral pathogenesis and the diversity of molecular pathways involved in SARS-CoV-2 infection.

Using a “minipool” CRISPR library of perturbations targeting top-ranked genes from the genome-scale CRISPR screen and single-cell transcriptomics, we identified a group of 6 genes (RAB7A, PIK3C3, NPC1, CCDC22, ATP6V1A, and ATP6AP1) that had a similar transcriptional signature-upregulation of the cholesterol synthesis pathway. We have recently, in an independent study, identified that SARS-CoV-2 infection negatively downregulates the cholesterol synthesis pathway and that viral infection can be counteracted by drug treatments that upregulate the same pathway (Hoagland et al., 2020). It is possible that changes in lipid composition directly impacts SARS-CoV-2 virion maturation and infectivity, as has been previously shown for Hepatitis C and Influenza A (Aizaki et al., 2008; Bajimaya et al., 2017). Some of the 6 genes have previously been implicated in regulating low-density lipoprotein (LDL) cholesterol. For example, depletion of Rab7a leads to LDL accumulation in endosomes and NPC1 knockout cells show a reduction of cholesterol at the plasma membrane and an accumulation in the late endosome/lysosome compartments (Chang et al., 2005; Girard et al., 2014; Millard et al., 2000; Neufeld et al., 1996). An important future research direction will be to further understand the relationship between cholesterol synthesis pathways and SARS-CoV-2.

Furthermore, we screened a panel of the top-ranked genes and identified that RAB7A regulates cell surface expression of ACE2, likely by sequestering ACE2 in endosomal vesicles. Rab7a is involved in vesicular trafficking and its depletion has been shown to sequester other cell receptors in endosomes (Rush and Ceresa, 2013). Interestingly, RAB7A knock-out cell lines showed both altered cholesterol biosynthesis and sequestration of ACE2 receptor. Also, previous proteomics work showed that Rab7a has a strong interaction with viral protein nsp7 (Gordon et al., 2020). However, there is no NSP7 in the incoming virion, implying a post-entry/post-translational role for RAB7A. Thus, it is possible that loss of RAB7A blocks SARS-CoV-2 pathogenesis via multiple separate pathways, which is supported by the observation that it is the top-performing gene in our arrayed validation.

We also sought to use our CRISPR screen to help interpret recent human genetic studies to identify putative causal genes for COVID-19 (COVID-19 Host Genetics Initiative, 2020). A pervasive problem in human genetic studies is that a majority of variation is in noncoding regions of the genome, making it challenging to understand mechanisms of action (ENCODE Project Consortium, 2012; Visel et al., 2009). Furthermore, this can make it challenging to identify causal variants and, even when identified, to map the genes through which these variants exert their biological effects (Sanjana et al., 2016). In our study, we first showed that eQTLs for top-ranked genes from the CRISPR screen are enriched for GWAS variants that correlate with COVID-19 severity. Then, we examined the locus with the strongest genome-wide signal for COVID-19 risk (3p21.31) and used our CRISPR screen results to identify 2 putative genes whose expression is modulated by these human genetic variants. Beyond COVID-19, this novel approach integrates colocalization analysis of eQTLs and GWAS variants with genome-scale loss-of-function CRISPR screens to bridge the divide between correlational and causal studies of human biology. This approach should prove useful for other human diseases and pathogens.

The methods and compositions described herein harnessed genome-scale loss-of-function to develop more refined therapeutic hypothesis and to identify certain drugs or compounds that may be useful in the inhibition of viral infection. In one embodiment, as shown herein, the methods and compositions identify PIK3C3 is a promising drug target. In the experiments described herein, four out of seven PIK3C3 inhibitors resulted in more than 100-fold reduction of SARS-CoV-2 viral load (SAR405, Compound-19, PIK-III, and Autophinib). Using a PIK3C3 polyclonal knockout A549ACE2 cell line, we found that among the top four PIK3C3 inhibitors, SAR405 may have some off-target effects (FIG. 10). In another embodiment, one of the drugs that shows a substantial reduction in SARS-CoV-2 viral load is tamoxifen. Tamoxifen is an FDA-approved drug given as prophylaxis to patients at risk of breast cancer and works via modulation of the estrogen receptor. Tamoxifen was included in our study as it targets protein kinase C as a secondary target (O'Brian et al., 1985). This mechanism is further supported by the observation that A549 cells have undetectable transcript levels of estrogen receptor 1 (Human Protein Atlas) (Uhlen et al., 2010). Considering that tamoxifen is typically given to patients for years as a cancer therapy and prophylactic (Marchant, 1976), it would be interesting to investigate if patients taking tamoxifen have a reduced risk of SARS-CoV-2 infection and/or display less severe symptoms post-infection.

Finally, many approaches for therapeutic discovery have focused on large-scale screens of compound libraries. Even when promising therapeutic candidates are identified, it can be challenging to understand the mechanisms responsible for reducing viral pathogenesis. Our forward-genetics approach allowed the inventors to first identify key host genes, which can then be targeted through a diversity of methods such as small-molecule inhibitors, blocking antibodies or gene knockdown. A key advantage of this approach is that the mechanism of action for any therapeutic is well-established from the outset. Taken together, this invention and the examples and figures herein identify essential host genes in SARS-CoV-2 viral pathogenesis and, through a broad range of analytic and experimental approaches, and validate their central role in infection. We identify mechanisms underlying top-ranked genes, including cholesterol synthesis and endosomal function, and use the high-throughput screen to further illuminate human genetic studies of COVID-19 risk and disease severity. In addition to providing new therapeutic targets to help end this pandemic, these methods and compositions provide a framework for harnessing massively-parallel genome editing to understand disease genetics and mechanisms.

In one aspect a method of treating, inhibiting, and/or preventing viral diseases comprising administering a composition that blocks or inhibits the expression, induction, activity, or signaling of one or more of one or a combination of the subject's genes required for viral infection. In one embodiment, the viral infection is caused by infection with the virus SARS-CoV-2. In certain embodiments, the methods involve administering to a mammalian subject (e.g., a human subject) in need thereof one or more inhibitors of the activity or expression of said genes or combination of such genes. The inhibitor can comprise a small molecule, a nucleic acid therapeutic, a peptide therapeutic, or another biologic therapeutic that mimics in the subject a loss of function of one or more of the selected genes.

### A. Subject

“Patient” or “subject” or “individual” as used herein means a mammalian animal, including a human, a veterinary or farm animal, a domestic animal or pet, and animals normally used for clinical research. In one embodiment, the subject of these methods and compositions is a human. In one embodiment, the subject has no viral infection but is in need of a prophylactic therapy. In another embodiment, the subject has a viral infection and has yet to be treated with any therapy. In another embodiment, the subject has a viral infection and is asymptomatic. In another embodiment, the subject has a viral infection, symptoms and is being treated with conventional methodologies, e.g., administration of currently available antivirals or anti-inflammatories, but is not responding to the treatment optimally or in a manner sufficient to achieve a sufficient therapeutic benefit. In another embodiment, the subject has a viral infection and is treated with conventional methodologies but is not achieving the desired therapeutically maximal response that been observed in other patients. In any of these embodiments, the viral infection is a SARS-CoV-2 infection.

### B. Human Genes or Combinations of Human Genes Required for Viral Infection

In one embodiment, the gene or combination is selected from the human genes identified in Table I. Table I is a list of the top 1000 human genes (i.e., top 5% of the CRISPR screen of ˜20,000 genes in the human genome) for both high MOI and low MOI SARS-CoV-2 virus screens as described herein. Each gene is identified with a ranking number as given by the RRA (Robust Rank Aggregation) algorithm and gene name for top 1000 genes in the low and high MOI screen. Inhibition of the expression or activity of these genes or their activity can retard or inhibit infection of the human cells by SARS-CoV-2. The genes are ranked by ability to prevent SARS-CoV-2 infection.

TABLE 1

| Rank | Top 1000 genes - low MOI | Top 1000 genes - high MOI |
|---|---|---|
| 1 | VPS35 | VPS35 |
| 2 | VPS29 | ATP6AP1 |
| 3 | SNX27 | SNX27 |
| 4 | ATP6AP1 | ATP6V0C |
| 5 | ATP6AP2 | ATP6V1G1 |
| 6 | ATP6V0C | ATP6V0B |
| 7 | COMMD3 | ACE2 |
| 8 | COMMD3-BMI1 | ATP6V1B2 |
| 9 | ATP6V1H | ATP6V0D1 |
| 10 | VPS26A | VPS29 |
| 11 | ACTR3 | SPEN |
| 12 | ACE2 | ACTR3 |
| 13 | ATP6V1G1 | ATP6V1H |
| 14 | ATP6V1C1 | ATP6AP2 |
| 15 | ATP6V1B2 | ATP6V1E1 |
| 16 | SPEN | TMEM199 |
| 17 | CCDC22 | ACTR2 |
| 18 | CTSL1 | RAB7A |
| 19 | RNF4 | COMMD3 |
| 20 | NUFIP1 | PPID |
| 21 | UVRAG | COMMD2 |
| 22 | HDAC9 | TOR1AIP1 |
| 23 | COMMD2 | ATP6V1A |
| 24 | HAL | PIK3C3 |
| 25 | KIAA1033 | COMMD3-BMI1 |
| 26 | ATP6V0B | CTSL1 |
| 27 | CTSL | VPS26A |
| 28 | IL17D | CCDC22 |
| 29 | CCDC93 | ARPC4-TTLL3 |
| 30 | COMMD10 | ATP6V1C1 |
| 31 | PIK3R4 | SLTM |
| 32 | SRSF1 | POLE2 |
| 33 | C11orf74 | WDR81 |
| 34 | ATP6V0D1 | ARPC3 |
| 35 | ACTR2 | CTSL |
| 36 | COL11A1 | C17orf50 |
| 37 | SOSTDC1 | C11orf96 |
| 38 | USP33 | LOC100289561 |
| 39 | RAB7A | UVRAG |
| 40 | COMMD4 | C10orf131 |
| 41 | PGBD4 | CNOT4 |
| 42 | SIAH1 | CCZ1B |
| 43 | CHST10 | ACP5 |
| 44 | ARPC4 | MRPS27 |
| 45 | CCDC74A | CHST14 |
| 46 | ETV1 | DPM3 |
| 47 | SAMD15 | ERMP1 |
| 48 | FUNDC2 | SFN |
| 49 | CCZ1B | COMMD4 |
| 50 | TNIP3 | MDFI |
| 51 | AP1G2 | RAET1G |
| 52 | ORAI2 | ATL1 |
| 53 | SLC38A8 | KRTAP9-6 |
| 54 | GEMIN2 | CAPN7 |
| 55 | CYB5R4 | S1PR5 |
| 56 | TMEM199 | CCDC115 |
| 57 | LRFN5 | EIF4A3 |
| 58 | ATPAF1 | KIAA1033 |
| 59 | PCDH17 | KCNIP2 |
| 60 | GNGT1 | NAT8 |
| 61 | STX7 | ARPC2 |
| 62 | SEMA6A | GNB2L1 |
| 63 | LLPH | ODF3 |
| 64 | C5orf55 | ATAD3C |
| 65 | MYRIP | SOCS1 |
| 66 | KIAA0319 | NRAP |
| 67 | NCAPG2 | NECAP2 |
| 68 | RNF187 | SMARCB1 |
| 69 | BCHE | FBXO27 |
| 70 | PPP1CB | LIN52 |
| 71 | RASA3 | KPNA4 |
| 72 | FAM211A | LGALS1 |
| 73 | SETD1B | CEP104 |
| 74 | ZNF639 | HNRNPC |
| 75 | PODN | NBPF4 |
| 76 | REV1 | NBPF6 |
| 77 | AFF2 | MCM3AP |
| 78 | CD1D | LPCAT2 |
| 79 | ARPC3 | OGG1 |
| 80 | GRID1 | LOC388813 |
| 81 | MBD6 | PCID2 |
| 82 | ZMYM6 | ARID1A |
| 83 | TMX2 | GOLPH3 |
| 84 | DNM2 | EPHX3 |
| 85 | TGFBR2 | NARS |
| 86 | SLC35B2 | C3orf33 |
| 87 | ZNF341 | AK6 |
| 88 | PPP3R1 | ARPC4 |
| 89 | NXN | C1orf63 |
| 90 | MAP3K3 | GFER |
| 91 | RBM24 | RNF125 |
| 92 | ERI3 | THBD |
| 93 | ARF5 | TNKS |
| 94 | ZNF107 | hsa-let-7i |
| 95 | ACTRT1 | MBD6 |
| 96 | MRPS23 | CLDN14 |
| 97 | BARD1 | C1orf68 |
| 98 | RAP1B | UBC |
| 99 | WDR7 | HIST1H3G |
| 100 | POLE | OR2T11 |
| 101 | OR10A6 | WBSCR17 |
| 102 | NUPR1 | OR5D13 |
| 103 | WDTC1 | SEC22A |
| 104 | CERS1 | SRD5A1 |
| 105 | WDR91 | KCTD3 |
| 106 | SERPINB9 | CCDC71L |
| 107 | LPPR5 | PRMT7 |
| 108 | OR5M1 | CCDC93 |
| 109 | SRSF6 | STX7 |
| 110 | ARSD | BOC |
| 111 | OR5T1 | SDPR |
| 112 | TMEM117 | DEPDC1B |
| 113 | HNRNPC | KIAA1683 |
| 114 | MYF6 | AGXT |
| 115 | COG2 | KLHL28 |
| 116 | CA6 | SF1 |
| 117 | PGD | CCDC77 |
| 118 | FAP | FBXO33 |
| 119 | DPF3 | THEM6 |
| 120 | WNT2 | PLXNB3 |
| 121 | IL20RA | CATSPERB |
| 122 | OR13J1 | KLC4 |
| 123 | OR2T33 | NPC1 |
| 124 | SLCO1A2 | SAP30L |
| 125 | C10orf131 | TBXAS1 |
| 126 | TMEM33 | MID2 |
| 127 | APCDD1 | SGOL1 |
| 128 | STK19 | SLC9A3R2 |
| 129 | GZMK | C9orf171 |
| 130 | BAX | OR6Q1 |
| 131 | SRFBP1 | SRXN1 |
| 132 | C17orf96 | RPS19BP1 |
| 133 | DNMT3B | SCGB2B2 |
| 134 | TECR | SLC29A4 |
| 135 | MCF2L | TMEM8C |
| 136 | BBS12 | WFDC9 |
| 137 | SH2D3A | NCOA5 |
| 138 | DEFA4 | TMEM82 |
| 139 | NAA16 | AGAP3 |
| 140 | PSD4 | SUSD4 |
| 141 | ADCY4 | EPPK1 |
| 142 | HAGHL | CALCOCO2 |
| 143 | ZNF878 | IL37 |
| 144 | WDR93 | BECN1 |
| 145 | DNAJB11 | FAM166B |
| 146 | AGAP3 | SLC37A1 |
| 147 | C22orf23 | OR6C1 |
| 148 | CCL13 | MRPS30 |
| 149 | IKZF4 | GLT8D1 |
| 150 | GNB2L1 | OTOS |
| 151 | ATP2C2 | NELL1 |
| 152 | CHKA | ADSS |
| 153 | DNAJB8 | ACAD10 |
| 154 | LAMP5 | TMED9 |
| 155 | WBP2 | POLR3A |
| 156 | HAND1 | SLC35A2 |
| 157 | LY96 | ACSL3 |
| 158 | DSTYK | MRPL37 |
| 159 | CKMT1B | VPS11 |
| 160 | C14orf166B | ADAD2 |
| 161 | FUS | SHMT1 |
| 162 | BRPF1 | ANKLE2 |
| 163 | ABCB4 | CENPV |
| 164 | NARS2 | IPO7 |
| 165 | AOX1 | DSTYK |
| 166 | ATP6V1A | FHL5 |
| 167 | RGP1 | SLC26A10 |
| 168 | XAGE2 | SLC39A10 |
| 169 | SEPT9 | AKT1S1 |
| 170 | ACTB | TMEM47 |
| 171 | CMTM2 | PRR19 |
| 172 | SLC41A2 | IL2 |
| 173 | ZBTB40 | CERS1 |
| 174 | BCL2 | HIGD1A |
| 175 | GBP1 | COG3 |
| 176 | SDR16C5 | CDK5 |
| 177 | SAPCD2 | SRSF6 |
| 178 | FAM167A | EZH1 |
| 179 | ARID1A | MAST4 |
| 180 | ATP5L2 | ZNF655 |
| 181 | LMLN | ALX3 |
| 182 | HIST1H2BJ | FUT5 |
| 183 | FAM90A1 | HES7 |
| 184 | C9orf156 | RSRC1 |
| 185 | MYB | WDR91 |
| 186 | NDUFV1 | EFCAB1 |
| 187 | CLEC16A | COG8 |
| 188 | KDM4E | CASP5 |
| 189 | ESYT1 | GPR15 |
| 190 | PLXNA1 | FITM1 |
| 191 | ZCCHC14 | MX2 |
| 192 | FOXH1 | NKX6-3 |
| 193 | EMILIN1 | TPR |
| 194 | ABO | VMA21 |
| 195 | ZNF24 | LARP7 |
| 196 | IL1RAPL2 | E2F3 |
| 197 | ZPBP | VPS54 |
| 198 | AGPAT9 | C1orf115 |
| 199 | KRTAP15-1 | C16orf3 |
| 200 | SMAD4 | PMFBP1 |
| 201 | SEPT2 | RPN2 |
| 202 | ACADS | SMC2 |
| 203 | GTDC1 | GRID1 |
| 204 | PDCD2L | FANCG |
| 205 | ITGA4 | KLHL8 |
| 206 | DECR1 | DEXI |
| 207 | FAIM | OR10Q1 |
| 208 | IL23A | WDR7 |
| 209 | FBXW2 | PTK6 |
| 210 | CPLX2 | ACVR1C |
| 211 | NDUFB2 | GALT |
| 212 | C20orf194 | SERPING1 |
| 213 | CDK3 | SLC46A1 |
| 214 | USP21 | MRGPRX3 |
| 215 | CENPI | VSTM2B |
| 216 | ZPLD1 | SMCR8 |
| 217 | ANKRD49 | MYH15 |
| 218 | RAB11FIP1 | TRIM40 |
| 219 | LOXHD1 | HACL1 |
| 220 | EMC9 | SSSCA1 |
| 221 | ATIC | KRT19 |
| 222 | LGALS9C | CEP97 |
| 223 | CEP128 | TMEM243 |
| 224 | NSUN4 | SNTN |
| 225 | CREBL2 | CABP2 |
| 226 | NRBF2 | GPR33 |
| 227 | CPT1B | SYCP1 |
| 228 | OR1C1 | ARHGAP19 |
| 229 | EIF3L | OLFML2A |
| 230 | TOP1 | NPAS1 |
| 231 | NAA30 | ZC3H7A |
| 232 | POU2AF1 | TMEM57 |
| 233 | PRR5L | ZBTB48 |
| 234 | CHRNG | AP2A2 |
| 235 | VN1R2 | ACOT6 |
| 236 | CCNT1 | RNF114 |
| 237 | MDGA1 | JUN |
| 238 | ATRIP | UBE2R2 |
| 239 | PLA2G12A | TEX33 |
| 240 | ORC6 | LNPEP |
| 241 | VTI1A | GMPPA |
| 242 | MAPK4 | MMP12 |
| 243 | WFDC13 | SH3RF3 |
| 244 | PRSS46 | FGFR4 |
| 245 | FGF7 | HDAC9 |
| 246 | C21orf58 | ARL8B |
| 247 | NXPE3 | OR2T4 |
| 248 | TMC1 | GAN |
| 249 | NME6 | ATOH1 |
| 250 | CTAGE4 | BCL7C |
| 251 | PPP1R12A | TANC2 |
| 252 | NXF1 | NVL |
| 253 | NUDT18 | EMILIN3 |
| 254 | ADPRH | DMBX1 |
| 255 | KRT18 | SMAD5 |
| 256 | ZNF442 | ENY2 |
| 257 | NTN5 | DEPDC5 |
| 258 | PSMB9 | PDSS1 |
| 259 | KCTD4 | PTRF |
| 260 | USF2 | KLK12 |
| 261 | VAC14 | DHX9 |
| 262 | TRIQK | C20orf166 |
| 263 | CCDC74B | BRK1 |
| 264 | TMSB4Y | ARHGAP35 |
| 265 | C9orf170 | C1orf52 |
| 266 | PTPN14 | DDX51 |
| 267 | DNM1 | UBE4A |
| 268 | ZFYVE16 | ZNF700 |
| 269 | REC8 | GNRH1 |
| 270 | TSPAN4 | COX8C |
| 271 | ETFB | C8B |
| 272 | PSMB4 | PRMT6 |
| 273 | TNFAIP8L1 | INO80E |
| 274 | TSTD2 | KCNG2 |
| 275 | SLC30A1 | SLC7A9 |
| 276 | HIF3A | SLC7A2 |
| 277 | C16orf78 | ZPLD1 |
| 278 | SYCP1 | VPS18 |
| 279 | ITPK1 | ZNF124 |
| 280 | SMYD2 | SLC6A9 |
| 281 | KCTD19 | WDR17 |
| 282 | TBCE | IQCH |
| 283 | RNF139 | TMPRSS3 |
| 284 | SHOC2 | MAP7D3 |
| 285 | PAQR4 | TM9SF2 |
| 286 | AQP6 | TRIM58 |
| 287 | EREG | PSPH |
| 288 | CERCAM | TCTEX1D4 |
| 289 | CD3EAP | EEF1B2 |
| 290 | DNMBP | OR4M2 |
| 291 | CBLN4 | EIF5B |
| 292 | HNRNPAB | ZNF300 |
| 293 | ACOX3 | IL12RB2 |
| 294 | FRY | WFDC13 |
| 295 | PHF19 | TNS1 |
| 296 | NAALAD2 | C16orf62 |
| 297 | VPS33A | CPLX3 |
| 298 | ZNF616 | EIF3I |
| 299 | BAP1 | C5orf34 |
| 300 | NOVA1 | TINAGL1 |
| 301 | PCCA | DSCAML1 |
| 302 | TNPO2 | SLC25A42 |
| 303 | S100A4 | C19orf24 |
| 304 | PPIH | GAMT |
| 305 | SPDL1 | RAD54L |
| 306 | CAPZA2 | FAM83G |
| 307 | STC2 | ABCF1 |
| 308 | WNT5A | ZNF322 |
| 309 | RAB17 | TNFAIP8L2-SCNM1 |
| 310 | IFNA17 | SOX14 |
| 311 | TMEM59 | CIDEC |
| 312 | IQSEC1 | SUB1 |
| 313 | CNTN1 | PSORS1C2 |
| 314 | KLHL30 | C8orf37 |
| 315 | ZNF641 | CPN2 |
| 316 | CCDC91 | TXK |
| 317 | ZNF521 | GNPTAB |
| 318 | UNC93A | MRPS24 |
| 319 | GMEB1 | DUS1L |
| 320 | PRRX2 | ASPDH |
| 321 | MTERFD1 | SRD5A3 |
| 322 | TMEM221 | F2R |
| 323 | COX6C | MTRNR2L8 |
| 324 | PRELID1 | CCL20 |
| 325 | SCGB1D1 | MIXL1 |
| 326 | TPBG | ZDBF2 |
| 327 | GAB3 | ATP6V1D |
| 328 | UBE2V2 | MAP3K11 |
| 329 | LRRFIP1 | MCM10 |
| 330 | OAZ2 | MFSD10 |
| 331 | DMTF1 | RARRES3 |
| 332 | SEC62 | UBE2V2 |
| 333 | ENC1 | AK3 |
| 334 | PPP1R11 | SETBP1 |
| 335 | PRAMEF1 | ZNF529 |
| 336 | C14orf169 | C8orf44-SGK3 |
| 337 | BCAR1 | PTCD1 |
| 338 | POU5F2 | PVRL3 |
| 339 | LARGE | SIGMAR1 |
| 340 | SLCO1B1 | SLC37A2 |
| 34 | KIAA1644 | ABCB10 |
| 342 | NCOA5 | PAX7 |
| 343 | FASLG | DDN |
| 344 | SCAPER | LRAT |
| 345 | IFNE | GLTP |
| 346 | CHST12 | NARFL |
| 347 | DPP6 | FAM120B |
| 348 | ADAD1 | CCDC64 |
| 349 | ARHGAP28 | LEPRE1 |
| 350 | RGPD3 | RTP1 |
| 351 | ENY2 | LDHB |
| 352 | ELF5 | COPZ2 |
| 353 | MRPS17 | PROSER2 |
| 354 | PSMC4 | CATSPER4 |
| 355 | SPANXN2 | CCT4 |
| 356 | CUTC | PRKCA |
| 357 | GATA1 | ARPP19 |
| 358 | FCAMR | SNRNP200 |
| 359 | CPS1 | GPR12 |
| 360 | ANTXR2 | SLC35B2 |
| 361 | WDR27 | COMMD10 |
| 362 | PFKM | LRRN2 |
| 363 | FAM127C | C10orf82 |
| 364 | HRAS | IL12A |
| 365 | DRD1 | PPAPDC1B |
| 366 | ZNF624 | PGD |
| 367 | EPSTI1 | ZNF626 |
| 368 | CCDC138 | KRTAP21-3 |
| 369 | PSMA3 | SYNE1 |
| 370 | CXCL5 | C8orf48 |
| 371 | H1FOO | KLK14 |
| 372 | CYBB | PKNOX1 |
| 373 | TMEM11 | GLTPD1 |
| 374 | WDR26 | KIF5C |
| 375 | PRKCSH | LIMA1 |
| 376 | SPERT | UBXN2B |
| 377 | SNX12 | HSPBAP1 |
| 378 | C16orf62 | MRPL20 |
| 379 | LGALSL | GLUD2 |
| 380 | ZNF607 | ACTG2 |
| 381 | C1orf173 | NME8 |
| 382 | NBPF4 | SGCZ |
| 383 | NBPF6 | DIAPH2 |
| 384 | CHAF1A | GTPBP4 |
| 385 | FRAT1 | SLCO2A1 |
| 386 | CCNA1 | AP4B1 |
| 387 | PAQR9 | CD81 |
| 388 | MMP9 | ZNF382 |
| 389 | TMEM186 | BCDIN3D |
| 390 | RAD18 | ZNF331 |
| 391 | PDE6A | C21orf2 |
| 392 | C17orf51 | SH3BGRL |
| 393 | CXCL1 | ATL2 |
| 394 | DYRK2 | ZNF414 |
| 395 | NUDT10 | POLR3C |
| 396 | GPR155 | HBQ1 |
| 397 | ZNF304 | PLBD1 |
| 398 | AFF4 | PTCD2 |
| 399 | BCL10 | BAX |
| 400 | CWC15 | COG4 |
| 401 | SNX22 | ZNF641 |
| 402 | AIM2 | MORC1 |
| 403 | ABCC11 | C19orf53 |
| 404 | RNF223 | WHSC1 |
| 405 | CCDC24 | MRPS23 |
| 406 | GPR113 | TGFB2 |
| 407 | TTC27 | BAD |
| 408 | PHF20 | MOB3B |
| 409 | CHCHD10 | ATF5 |
| 410 | WBP4 | FCAMR |
| 411 | TRMT112 | REG1A |
| 412 | UBQLNI | PHTF1 |
| 413 | IFI16 | CHD5 |
| 414 | WDR81 | ENTHD1 |
| 415 | AGL | POC1A |
| 416 | MAP3K10 | PHF3 |
| 417 | OSM | TMEM147 |
| 418 | RPL39 | C9orf89 |
| 419 | C4BPA | CCDC92 |
| 420 | ZDHHC4 | TNFRSF12A |
| 421 | GNAS | UNC5D |
| 422 | SOAT1 | TPCN1 |
| 423 | C12orf75 | HIST1H3H |
| 424 | ANO6 | HSD17B8 |
| 425 | ZCCHC16 | DSCR3 |
| 426 | ZFHX2 | ALG14 |
| 427 | CCDC151 | TTLL1 |
| 428 | MYO7B | GPR116 |
| 429 | UNC5C | SHCBP1L |
| 430 | GNPTAB | TMEM105 |
| 431 | STON1-GTF2A1L | MYO7B |
| 432 | WDR48 | PRSS33 |
| 433 | MTRF1 | MXRA5 |
| 434 | CCDC130 | COX17 |
| 435 | HYAL2 | EEF2 |
| 436 | SYNC | NUPR1 |
| 437 | MYBL1 | IQSEC1 |
| 438 | ELMO1 | UPK3BL |
| 439 | THTPA | CLEC16A |
| 440 | HTN1 | SLC6A16 |
| 441 | ZC3H18 | MYL6 |
| 442 | GDAP2 | PEX11G |
| 443 | RBPMS | ARR3 |
| 444 | BCORL1 | ZNF273 |
| 445 | DDX39A | SPTBN5 |
| 446 | TYW5 | LCAT |
| 447 | LAMTOR5 | RNF183 |
| 448 | RNF141 | NAGPA |
| 449 | KCNIP3 | OR8K3 |
| 450 | HTR1F | SLC35A1 |
| 451 | CLEC19A | NUTF2 |
| 452 | TMEM50A | TCEANC |
| 453 | TUSC2 | ABHD6 |
| 454 | SLC25A15 | MARK1 |
| 455 | UVSSA | SPOCK3 |
| 456 | NR0B1 | EREG |
| 457 | ADSS | PLRG1 |
| 458 | GALNT14 | ZNF644 |
| 459 | TEX101 | POPDC3 |
| 460 | ENOSF1 | PRMT8 |
| 461 | SAMD4A | FUT2 |
| 462 | GOLGB1 | C1orf210 |
| 463 | COL17A1 | FAM162A |
| 464 | ZCCHC4 | OPN1LW |
| 465 | VMA21 | SPRR1B |
| 466 | SMPD3 | BRDT |
| 467 | OFD1 | NTMT1 |
| 468 | SNAPC4 | GPR82 |
| 469 | TBC1D10C | CLCN6 |
| 470 | HECW2 | GPATCH1 |
| 471 | PLEKHA3 | ACKR2 |
| 472 | GPER | ZSCAN30 |
| 473 | DRC1 | DDX18 |
| 474 | TCHP | PSD3 |
| 475 | HLA-DRA | MGAM |
| 476 | ISLR | ARRDC3 |
| 477 | NOL12 | KHDRBS2 |
| 478 | SLC18A2 | TMEM41B |
| 479 | RTN1 | AAK1 |
| 480 | DPP9 | SLC16A14 |
| 481 | IL26 | ANKRD36 |
| 482 | RGL1 | RAB11FIP1 |
| 483 | KIAA1432 | LECT1 |
| 484 | TMEM134 | LRRC8D |
| 485 | CELA2A | DSE |
| 486 | PPP1R15A | FAM134A |
| 487 | EIF3G | NR2F6 |
| 488 | C3orf62 | ACRV1 |
| 489 | TGFBR3 | TNS4 |
| 490 | PLEKHS1 | CFHR1 |
| 491 | PTF1A | PRAMEF22 |
| 492 | LTBP2 | ROPN1L |
| 493 | IL1F10 | EPOR |
| 494 | KIF3A | OTUD7B |
| 495 | C3orf18 | DCAF13 |
| 496 | FAM78B | B3GALT2 |
| 497 | GMNC | KCNA4 |
| 498 | ATP13A4 | COG7 |
| 499 | INSIG2 | FAM21B |
| 500 | SPTAN1 | SSH1 |
| 501 | HYAL4 | RTF1 |
| 502 | ITGB1BP1 | AIM2 |
| 503 | SEMA7A | C1QTNF7 |
| 504 | SLC3A2 | PLGRKT |
| 505 | RLN3 | TRIM23 |
| 506 | INHBB | CXCR6 |
| 507 | PDSS1 | CER1 |
| 508 | XPO6 | CACNG2 |
| 509 | EPHA5 | CUL3 |
| 510 | WDR31 | HNF1A |
| 511 | MOB3B | RASAL1 |
| 512 | TIPARP | OSGIN1 |
| 513 | HSP90AB1 | GUK1 |
| 514 | ZNF175 | WDR20 |
| 515 | UBAP2 | TSHZ2 |
| 516 | MICALL2 | RANBP9 |
| 517 | SRSF3 | CPB1 |
| 518 | ZNF182 | ST3GAL3 |
| 519 | RBM20 | TMEM179B |
| 520 | MKX | SOCS7 |
| 521 | BARX2 | FDPS |
| 522 | PARK7 | GSKIP |
| 523 | PUM1 | ADARB1 |
| 524 | TMEM5 | DBF4B |
| 525 | GPR68 | OR10AG1 |
| 526 | VWA2 | LAIR2 |
| 527 | FAM73A | COL3A1 |
| 528 | CREBZF | GOLGB1 |
| 529 | ASB16 | FAP |
| 530 | USP9Y | ALPP |
| 531 | TTI2 | ZNF227 |
| 532 | RELT | ITPRIPL1 |
| 533 | GFER | ALDH18A1 |
| 534 | TMEM119 | RPA1 |
| 535 | STK17A | C11orf21 |
| 536 | TEX28 | FBLN5 |
| 537 | GFOD1 | SLC16A3 |
| 538 | RAB1A | HMGB4 |
| 539 | TGIF1 | SPATA31A6 |
| 540 | SKA3 | TSC1 |
| 541 | LOXL4 | INHBC |
| 542 | KRT80 | SNTA1 |
| 543 | ICA1 | PCSK9 |
| 544 | FOXO3 | VSIG1 |
| 545 | VARS2 | TP53BP2 |
| 546 | BLOC1S1 | ADCK1 |
| 547 | LMO4 | NIPSNAP3A |
| 548 | PHF3 | CDC42EP1 |
| 549 | HIST1H3B | XPNPEP2 |
| 550 | PRLHR | PSMC3IP |
| 551 | TSHZ1 | MAMLD1 |
| 552 | OR6C1 | CTIF |
| 553 | ERCC1 | RAB34 |
| 554 | TRIM50 | FCRLA |
| 555 | HEPH | GPR158 |
| 556 | SPRR4 | MRI1 |
| 557 | RNF20 | NUMB |
| 558 | KIAA0930 | RBKS |
| 559 | GLIPR1L2 | PCNXL3 |
| 560 | HDAC7 | ACYP2 |
| 561 | TFAM | CCND1 |
| 562 | CRLF1 | PLOD1 |
| 563 | C11orf16 | ANKRD10 |
| 564 | LEO1 | OR6A2 |
| 565 | ZFP36L2 | B3GNT9 |
| 566 | WDSUB1 | NXF1 |
| 567 | TRIM52 | GALE |
| 568 | TNRC6A | PLBD2 |
| 569 | XKR9 | PRKD2 |
| 570 | TBC1D8 | C19orf60 |
| 571 | PPAT | ZNF671 |
| 572 | GP5 | S1PR2 |
| 573 | DUX2 | DYRK3 |
| 574 | MYOCD | BPIFA2 |
| 575 | RSPH10B2 | ECM2 |
| 576 | DNAAF3 | NGRN |
| 577 | SLC9A7 | H3F3C |
| 578 | ACVR1 | CHEK2 |
| 579 | HBS1L | LSM6 |
| 580 | PCP4 | CPM |
| 58 | TGIF2LX | UCK2 |
| 582 | PDZRN4 | AMPD2 |
| 583 | CCDC126 | ZNF253 |
| 584 | KRTAP9-8 | OR8B3 |
| 585 | PTGER1 | TROVE2 |
| 586 | ADRA1D | CCNI |
| 587 | PAQR5 | EPB41 |
| 588 | MAP7D3 | GJA4 |
| 589 | KRTCAP2 | SWSAP1 |
| 590 | C1orf63 | SRSF3 |
| 59 | FSD1L | SPDYA |
| 592 | OR10G7 | HOXD13 |
| 593 | IFNL1 | MS4A10 |
| 594 | ZFAND2B | EYA3 |
| 595 | POLR3A | KIF2C |
| 596 | CAPG | ETV1 |
| 597 | STK31 | GUSB |
| 598 | IQCA1 | TOMM40L |
| 599 | B4GALNT1 | ZMYND8 |
| 600 | OR4A16 | HP1BP3 |
| 601 | GJB3 | IL27RA |
| 602 | SAMD5 | AGER |
| 603 | CTDSP2 | NUP50 |
| 604 | FNDC3B | SNX14 |
| 605 | EPPK1 | TMEM55B |
| 606 | ACSF3 | BCAN |
| 607 | CAPZA1 | NAA11 |
| 608 | KLHL3 | MSANTD2 |
| 609 | FBXL13 | DACT3 |
| 610 | ARL1 | OTUD5 |
| 611 | CLEC1B | FAM131A |
| 612 | LRRC8D | STRA8 |
| 613 | CYP19A1 | DCAF8L1 |
| 614 | CCDC92 | NOX4 |
| 615 | TTC4 | OR5R1 |
| 616 | FCHO1 | TXLNA |
| 617 | SLC37A1 | GSTO2 |
| 618 | PSMD1 | RHOJ |
| 619 | RAP1GDS1 | PVALB |
| 620 | IFRD1 | NEFL |
| 621 | TFAP4 | CLASP2 |
| 622 | RNASE1 | CDKN1C |
| 623 | HELLS | CNNM3 |
| 624 | CXXC11 | C1orf94 |
| 625 | OR8G2 | HOXD12 |
| 626 | ACTG2 | C22orf39 |
| 627 | NDUFA8 | FNDC1 |
| 628 | PEX1 | ELOVL1 |
| 629 | GPR17 | AREL1 |
| 630 | PGLYRP1 | MIA2 |
| 631 | FAM135B | TMEM86A |
| 632 | OR4D1 | CLTA |
| 633 | ARHGAP31 | USP28 |
| 634 | CDR2 | MILR1 |
| 635 | KIAA0430 | NFKBIL1 |
| 636 | MORN1 | GSTCD |
| 637 | TEX13B | TNFSF18 |
| 638 | SOCS7 | ARHGAP22 |
| 639 | C4orf17 | EPS8L3 |
| 640 | AP3M1 | CCT8L2 |
| 641 | SUGP2 | RPL11 |
| 642 | PTP4A3 | KLHL9 |
| 643 | RAD51B | PNMA2 |
| 644 | EPHB3 | TRAPPC12 |
| 645 | DTX4 | FAM122B |
| 646 | NINL | SPRED3 |
| 647 | TRPC7 | MPST |
| 648 | PRKCG | PRKAA1 |
| 649 | NFKBIL1 | WDR67 |
| 650 | PSMD9 | PDCD1 |
| 651 | OR6K3 | PGPEP1 |
| 652 | RPA1 | CHMP4C |
| 653 | A2ML1 | RNF139 |
| 654 | SSPO | TUSC3 |
| 655 | NCOA3 | CYP11B2 |
| 656 | CRISP3 | GPX3 |
| 657 | TAAR6 | INPP4B |
| 658 | STT3B | FOXI3 |
| 659 | C7orf71 | SPATA12 |
| 660 | CENPV | GPR182 |
| 661 | KCNQ4 | HPDL |
| 662 | CSF1R | TTC21B |
| 663 | TGFBR3L | IKBKAP |
| 664 | TIE1 | SGCG |
| 665 | SWSAP1 | TTC25 |
| 666 | BECN1 | NUFIP1 |
| 667 | NMUR1 | ZBTB40 |
| 668 | C1orf61 | DCUN1D4 |
| 669 | ZMYND10 | C11orf83 |
| 670 | OR8B4 | OR2B11 |
| 671 | FMNL2 | IGFBP3 |
| 672 | ZNF529 | ANTXR2 |
| 673 | ME2 | PI3 |
| 674 | HMG20B | HTRA3 |
| 675 | FOXRED1 | EDARADD |
| 676 | DSE | QPRT |
| 677 | GABARAPL2 | ZBTB8B |
| 678 | FREM3 | ETV7 |
| 679 | ACER3 | SEC31B |
| 680 | SNX15 | AMDHD2 |
| 681 | FCHO2 | HTR1E |
| 682 | RBFOX2 | CPNE6 |
| 683 | KIAA1109 | SYNGAP1 |
| 684 | ANKRD29 | CCDC85A |
| 685 | NET1 | ZBTB21 |
| 686 | NUP205 | FER |
| 687 | VPS36 | FAM175A |
| 688 | APAF1 | SLC25A33 |
| 689 | NPAS3 | ZNF101 |
| 690 | CCDC169-SOHLH2 | EPHX1 |
| 691 | LSM6 | A3GALT2 |
| 692 | CBR4 | DNAL4 |
| 693 | ARPC4-TTLL3 | KIF2A |
| 694 | IQCC | ALG6 |
| 695 | SDK1 | PSEN1 |
| 696 | SLC25A23 | AATF |
| 697 | FBXL5 | MORN3 |
| 698 | RAB39B | HHIPL2 |
| 699 | CASP8 | TMEM110-MUSTN1 |
| 700 | TMEM161A | KLHL15 |
| 701 | TF | TRPC4 |
| 702 | SGCB | MAPK8IP2 |
| 703 | LCE5A | GHDC |
| 704 | LGI1 | PPP1CB |
| 705 | NLK | WDR16 |
| 706 | HECW1 | BCORL1 |
| 707 | VEZT | MLKL |
| 708 | NANOS2 | LIPC |
| 709 | MOS | HIST1H2BH |
| 710 | MTPN | TBCEL |
| 711 | TNKS2 | WNT16 |
| 712 | FAM104A | ZNF248 |
| 713 | SLC28A1 | CDRT15 |
| 714 | CEP68 | CNTROB |
| 715 | OR51A4 | KLKB1 |
| 716 | PRKRIP1 | RBM15 |
| 717 | NDUFB6 | TMC4 |
| 718 | PPAPDC1A | TRMT112 |
| 719 | SMOX | CACNA2D1 |
| 720 | BCAS2 | C2CD3 |
| 721 | OR8K3 | PARG |
| 722 | PLCXD3 | HSDL1 |
| 723 | DENND5B | OR5B2 |
| 724 | TP53BP2 | KCNE1 |
| 725 | HRH4 | SLC25A31 |
| 726 | OGG1 | NDUFA3 |
| 727 | RAB38 | ANKRD54 |
| 728 | TSPAN8 | SLC1A4 |
| 729 | CYB5R2 | GAL3ST2 |
| 730 | BRSK2 | VPS41 |
| 731 | CCDC107 | TRAK1 |
| 732 | TAF8 | WDR18 |
| 733 | HILPDA | PSTPIP1 |
| 734 | NDN | MEOX1 |
| 735 | ZNF45 | NIT2 |
| 736 | IL27 | TLE3 |
| 737 | UPK1B | TSPAN9 |
| 738 | KMT2C | RPS6KA4 |
| 739 | MFSD8 | MTRNR2L2 |
| 740 | NRTN | TIPARP |
| 741 | PARP9 | EPHB4 |
| 742 | NEURL1B | NPAP1 |
| 743 | ARNT | PRAF2 |
| 744 | PRRC1 | IL27 |
| 745 | ST3GAL6 | QPCTL |
| 746 | BHMT | PCK1 |
| 747 | TEAD2 | POLR1D |
| 748 | ZNF567 | AGTR1 |
| 749 | PPID | SNAP47 |
| 750 | SKOR1 | FAM196B |
| 751 | ARSF | SON |
| 752 | ODF3L2 | CYP4F12 |
| 753 | HLA-F | CS |
| 754 | TCEA1 | MALL |
| 755 | C9orf171 | GPD1L |
| 756 | RHOBTB2 | ARAP2 |
| 757 | C4orf51 | DNAJC5 |
| 758 | ZNF574 | COX6C |
| 759 | HIF1A | AADAT |
| 760 | LINGO2 | HBE1 |
| 761 | MIEN1 | ATP5J2-PTCD1 |
| 762 | DPYSL4 | UBFD1 |
| 763 | NEK1 | COX5B |
| 764 | GOLGA2 | SLC10A5 |
| 765 | HIST3H2A | CASQ2 |
| 766 | TSPAN16 | TRA2B |
| 767 | NANOS3 | BHLHE41 |
| 768 | TMEM9B | ZNF792 |
| 769 | F7 | ENAH |
| 770 | PASK | HUS1 |
| 771 | OR8J1 | IL17D |
| 772 | TNFSF12 | PRKRIR |
| 773 | HOXB3 | MAFB |
| 774 | FAM83C | KCTD8 |
| 775 | F13A1 | COX6B1 |
| 776 | OMA1 | MAX |
| 777 | FCN1 | LIMS2 |
| 778 | SURF4 | CD7 |
| 779 | FABP3 | CCDC68 |
| 780 | ZSCAN30 | SIRPG |
| 781 | KLK10 | NODAL |
| 782 | OR5W2 | WDR47 |
| 783 | ERGIC1 | CCL13 |
| 784 | PSMC3 | IFNE |
| 785 | COPRS | GIMAP1 |
| 786 | ATL1 | HIPK3 |
| 787 | ERF | LYRM2 |
| 788 | ATG5 | TRUB1 |
| 789 | MEF2BNB | LAMC3 |
| 790 | CAPN5 | TECR |
| 791 | C11orf49 | CCIN |
| 792 | LIPT1 | DIDO1 |
| 793 | MAP4K3 | ERAP2 |
| 794 | BSND | SNRPC |
| 795 | CUZD1 | C2orf57 |
| 796 | CXorf23 | CAMK2D |
| 797 | PSME3 | C20orf85 |
| 798 | C12orf56 | PIWIL3 |
| 799 | UBE2R2 | LMAN1L |
| 800 | SYT3 | SOX12 |
| 801 | PDAP1 | NDC1 |
| 802 | ASGR2 | OR10Z1 |
| 803 | RABL2B | LIN28B |
| 804 | HS2ST1 | STARD13 |
| 805 | AP4S1 | EMC10 |
| 806 | HAP1 | AMOTL2 |
| 807 | SLC26A8 | WISP3 |
| 808 | BFSP1 | KIAA0100 |
| 809 | PSD2 | SMIM10 |
| 810 | GLIS3 | RNF34 |
| 811 | VIL1 | RTN4R |
| 812 | CALCOCO2 | PRR9 |
| 813 | HMBS | HTR1A |
| 814 | OCEL1 | LGALS7B |
| 815 | ARHGAP17 | VSIG2 |
| 816 | RHCE | SLC27A2 |
| 817 | HRASLS | MTHFD1L |
| 818 | DCX | LDOC1 |
| 819 | G6PC | TAF1L |
| 820 | ZNF503 | KLHL35 |
| 821 | FOXD4 | REV1 |
| 822 | ANK2 | SPSB4 |
| 823 | GNRH1 | IRX3 |
| 824 | GPR153 | HOXA3 |
| 825 | SAE1 | PTGES |
| 826 | KXD1 | CT62 |
| 827 | NOA1 | ZNF195 |
| 828 | LAIR1 | EBF2 |
| 829 | PSG9 | CCDC102B |
| 830 | MRI1 | NKAPL |
| 831 | FFAR4 | PDF |
| 832 | FRA10AC1 | VTI1B |
| 833 | ALX4 | LIG3 |
| 834 | PRKRIR | OVOL3 |
| 835 | IL37 | PPP3CB |
| 836 | ANXA8 | ENSA |
| 837 | PGM5 | KCNJ11 |
| 838 | NME8 | SPEF2 |
| 839 | COX10 | PRKX |
| 840 | LRCH1 | SPACA3 |
| 841 | GOLGA7B | DERL1 |
| 842 | MRPL47 | LRRC20 |
| 843 | YTHDF1 | SLC26A1 |
| 844 | NTRK1 | RBM18 |
| 845 | SLC25A51 | UFM1 |
| 846 | RCBTB2 | CHADL |
| 847 | KRT16 | ADAM10 |
| 848 | SOX21 | FAM46D |
| 849 | CD164 | CD8B |
| 850 | CD3G | ARHGEF18 |
| 851 | MPL | SFTPA1 |
| 852 | TMPRSS3 | SCARF2 |
| 853 | TPGS2 | SLC25A5 |
| 854 | MYH9 | DOK7 |
| 855 | C10orf2 | LRFN3 |
| 856 | RAB39A | MFAP3L |
| 857 | MPPED1 | TMEM117 |
| 858 | KCNS3 | ALOX15 |
| 859 | TARS | BFSP1 |
| 860 | TPP2 | ADCK2 |
| 861 | SGMS2 | POU3F2 |
| 862 | KCNE2 | BPIFB2 |
| 863 | MRPS34 | NYAP2 |
| 864 | MTRNR2L8 | TST |
| 865 | NF2 | TSGA10 |
| 866 | PTPN6 | RAB3D |
| 867 | GMEB2 | TNNT3 |
| 868 | C3 | SCEL |
| 869 | FZR1 | CSNK2A3 |
| 870 | OR3A2 | GAB4 |
| 871 | DDX55 | CD96 |
| 872 | CLK4 | ALDH16A1 |
| 873 | CHGB | TPD52L1 |
| 874 | THSD7B | USP35 |
| 875 | MFSD9 | ARL15 |
| 876 | PCK1 | TAS2R14 |
| 877 | FAM71C | TRHDE |
| 878 | METTL3 | DOCK6 |
| 879 | TAF11 | OR56A5 |
| 880 | MDK | NKD1 |
| 881 | DYDC2 | RNASE1 |
| 882 | FAM21A | ARFRP1 |
| 883 | HORMAD2 | LATS2 |
| 884 | C10orf82 | RABL2B |
| 885 | CHCHD7 | FZD8 |
| 886 | GBP4 | ECI1 |
| 887 | OGN | ANKRD50 |
| 888 | SRPK2 | USP50 |
| 889 | FDX1 | DCST1 |
| 890 | CTNNB1 | NUDCD3 |
| 891 | PARPBP | GDF1 |
| 892 | C1orf233 | VPS13D |
| 893 | TRIM31 | TIGD5 |
| 894 | CEP63 | ITGB1BP1 |
| 895 | POGLUT1 | RRAGA |
| 896 | KLHL5 | TIGD1 |
| 897 | AGBL3 | KPNA7 |
| 898 | RNF39 | GPR17 |
| 899 | ZNF749 | COX4I1 |
| 900 | COQ3 | PPIAL4F |
| 901 | PLEK | TUBB2B |
| 902 | TRIP11 | FMN2 |
| 903 | DEXI | SMIM12 |
| 904 | CCDC168 | HS2ST1 |
| 905 | MRPS26 | NPY4R |
| 906 | ANKRD33B | REEP4 |
| 907 | KIAA1191 | CDKN2D |
| 908 | TUT1 | SLC38A8 |
| 909 | ETV5 | ANG |
| 910 | BCL2L2 | ZBED1_X |
| 911 | CD97 | ZBED1_Y |
| 912 | GAL3ST4 | CA5B |
| 913 | EPOR | ENTPD4 |
| 914 | OR9I1 | OR2C1 |
| 915 | PMCH | CHPF |
| 916 | ITGAE | TRIM33 |
| 917 | BTBD18 | STEAP1 |
| 918 | CIDEC | NUAK2 |
| 919 | GBE1 | LAMP5 |
| 920 | ACADM | OR2T35 |
| 921 | CLIP3 | POLD1 |
| 922 | HEY2 | COL9A2 |
| 923 | HTRA2 | RSPH1 |
| 924 | ROCK2 | FOLR3 |
| 925 | EHD3 | BRWD1 |
| 926 | PECR | BCL2L13 |
| 927 | PTPDC1 | MOB3C |
| 928 | PLEKHG6 | LRRC15 |
| 929 | C2orf44 | GH1 |
| 930 | RRAD | TNFSF12 |
| 931 | OR10J5 | CDT1 |
| 932 | IL17RA | ZNF556 |
| 933 | BAD | SH3BP1 |
| 934 | STK33 | RTN3 |
| 935 | HES3 | STEAP2 |
| 936 | PLXDC2 | NF2 |
| 937 | SNAP25 | TMEM63C |
| 938 | MBL2 | KIAA1191 |
| 939 | CCNB1 | KRTAP4-2 |
| 940 | FMR1 | DFFA |
| 941 | ALG9 | CSF3R |
| 942 | STX5 | PCNT |
| 943 | LOC100507003 | NFATC2 |
| 944 | SF1 | FGF22 |
| 945 | ACAT1 | C12orf23 |
| 946 | TKTL2 | C10orf95 |
| 947 | HOMEZ | PPP1R35 |
| 948 | ANKZF1 | EYA2 |
| 949 | PPP1R3C | KIAA1467 |
| 950 | CNTNAP2 | CPA3 |
| 951 | LPIN1 | HEBP1 |
| 952 | DDX59 | EPHA3 |
| 953 | KATNBL1 | TSPAN11 |
| 954 | PDHA2 | C4orf46 |
| 955 | FAM98C | GBE1 |
| 956 | CCDC51 | CORO2A |
| 957 | FBXO46 | NRN1L |
| 958 | GPR56 | PRAMEF15 |
| 959 | TRIM6-TRIM34 | PRAMEF9 |
| 960 | SRPX2 | RRAGB |
| 961 | SMAGP | MAN2B1 |
| 962 | SLC12A1 | SNX12 |
| 963 | TRIP10 | CHCHD10 |
| 964 | LSM2 | GCOM1 |
| 965 | RERGL | MYZAP |
| 966 | MMADHC | ME2 |
| 967 | USP40 | TRIP11 |
| 968 | FAM149A | WAC |
| 969 | WDR49 | RASL10B |
| 970 | SIGLEC5 | CDC25A |
| 971 | OR51V1 | AMBN |
| 972 | NEU4 | HIST1H2AM |
| 973 | CYP2J2 | ZNF345 |
| 974 | C14orf182 | RPTOR |
| 975 | FDX1L | TNNC2 |
| 976 | NDUFC2 | TIMM9 |
| 977 | TMEM167B | ACBD5 |
| 978 | TUSC5 | FAM96A |
| 979 | C7orf73 | TMEM185A |
| 980 | RGAG1 | FAM21C |
| 981 | PRR21 | AHNAK2 |
| 982 | CACNG6 | SPHK2 |
| 983 | ZNF74 | POGZ |
| 984 | PTPRF | GPR52 |
| 985 | LOC389895 | RGS13 |
| 986 | TRIM72 | FRMD8 |
| 987 | ITPRIPL1 | MED29 |
| 988 | SSX1 | NCBP1 |
| 989 | TCEANC | PTK2B |
| 990 | S100A9 | ABHD8 |
| 991 | DHX57 | LAPTM5 |
| 992 | MPZL2 | KRTAP4-4 |
| 993 | CBX8 | GABRQ |
| 994 | OR10W1 | RPS6KL1 |
| 995 | NKX2-5 | CD320 |
| 996 | LOC100130357 | MARVELD2 |
| 997 | ZNF354C | SCAF8 |
| 998 | LOC286238 | RGS2 |
| 999 | ACAP3 | ARX |
| 1000 | PINK1 | SEMA6A |

In one embodiment, the gene or combination is selected from the genes identified in Table II. Table II is a list of the 69 (low MOI SARS-CoV-2 screen) or 73 (high MOI SARS-CoV-2 screen) genes that were identified among the top 500 genes ranked in the CRISPR screens described herein. These genes were among the top 500 in each screen for which drugs that inhibit their expression or activity are useful in retarding or inhibiting infection of the human cells by SARS-CoV-2. Among these are 10 genes that overlapped in both screens. The genes are ranked by ability to prevent SARS-CoV-2 infection as given in the ranked lists.

TABLE 2

| | Low MOI | High MOI | |
|---|---|---|---|
| Rank | Druggable Genes | Druggable Genes | Common |
| 1 | ACE2 | ACE2 | ACE2 |
| 2 | ACTR2 | HDAC9 | ACTR2 |
| 3 | PPID | CTSL | ATP6V1A |
| 4 | ATP6V1A | PIK3R4 | CTSL |
| 5 | PIK3C3 | ACTR2 | ARID1A |
| 6 | CTSL | COL11A1 | ADSS |
| 7 | ACP5 | SIAH1 | HDAC9 |
| 8 | S1PR5 | ETV1 | PGD |
| 9 | SOCS1 | BCHE | BAX |
| 10 | SMARCB1 | TGFBR2 | EREG |
| 11 | OGG1 | MAP3K3 | |
| 12 | ARID1A | POLE | |
| 13 | GFER | CA6 | |
| 14 | THBD | PGD | |
| 15 | SRD5A1 | FAP | |
| 16 | PRMT7 | SLCO1A2 | |
| 17 | TBXAS1 | BAX | |
| 18 | SLC29A4 | DEFA4 | |
| 19 | ADSS | CHKA | |
| 20 | IPO7 | LY96 | |
| 21 | AKT1S1 | FUS | |
| 22 | IL2 | BRPF1 | |
| 23 | CDK5 | ABCB4 | |
| 24 | EZH1 | AOX1 | |
| 25 | CASP5 | ATP6V1A | |
| 26 | E2F3 | ACTB | |
| 27 | PTK6 | BCL2 | |
| 28 | ACVR1C | ARID1A | |
| 29 | SERPING1 | MYB | |
| 30 | KRT19 | NDUFV1 | |
| 31 | JUN | KDM4E | |
| 32 | LNPEP | SMAD4 | |
| 33 | MMP12 | ITGA4 | |
| 34 | FGFR4 | IL23A | |
| 35 | HDAC9 | NDUFB2 | |
| 36 | GNRH1 | CDK3 | |
| 37 | PRMT6 | ATIC | |
| 38 | KCNG2 | CPT1B | |
| 39 | SLC6A9 | TOP1 | |
| 40 | IL12RB2 | CHRNG | |
| 41 | TXK | KRT18 | |
| 42 | SRD5A3 | PSMB9 | |
| 43 | F2R | PSMB4 | |
| 44 | MAP3K11 | SMYD2 | |
| 45 | SETBP1 | AQP6 | |
| 46 | SIGMAR1 | EREG | |
| 47 | CATSPER4 | BAP1 | |
| 48 | PRKCA | CNTN1 | |
| 49 | IL12A | TPBG | |
| 50 | PGD | SLCO1B1 | |
| 51 | KLK14 | FASLG | |
| 52 | SLCO2A1 | PSMC4 | |
| 53 | BAX | GATA1 | |
| 54 | TGFB2 | CPS1 | |
| 55 | BAD | HRAS | |
| 56 | REG1A | DRD1 | |
| 57 | TNFRSF12A | PSMA3 | |
| 58 | TPCN1 | MMP9 | |
| 59 | EEF2 | PDE6A | |
| 60 | LCAT | DYRK2 | |
| 61 | ABHD6 | MAP3K10 | |
| 62 | MARK1 | OSM | |
| 63 | EREG | GNAS | |
| 64 | PRMT8 | SOAT1 | |
| 65 | BRDT | HYAL2 | |
| 66 | CLCN6 | BCORL1 | |
| 67 | MGAM | HTR1F | |
| 68 | EPOR | ADSS | |
| 69 | KCNA4 | ENOSF1 | |
| 70 | | HLA-DRA | |
| 71 | | SLC18A2 | |
| 72 | | DPP9 | |
| 73 | | TGFBR3 | |

In one embodiment, the gene or combination is selected from the genes identified in Table III. Table III is a list of the top 69 genes identified in the low MOI SARS-CoV-2 screen and the corresponding drugs known to inhibit expression or activity of these genes. These genes are not ranked in the same order as for Table I or Table II. The drugs are listed as inhibiting the corresponding gene (see, e.g., the Drug Gene Interaction database: www.dgidb.org/). Previous reports have established that these small-molecule drugs can inhibit (“mimic loss-of-function”) of the corresponding gene in the list. That is, the drug copies loss-of-function/knock-out of the gene without the need for other means of knocking out gene expression or activity, such as CRISPR, genome editing, etc.

TABLE III

| Rank | Gene | Inhibitor |
|---|---|---|
| 1 | ABHD6 | WWL70 \| WWL123 |
| 2 | ACE2 | COMPOUND 28 [PMID: 18324760] \| XNT [PMID: 18391097] \| |
| | | MLN-4760 \| GL1001 \| ABZ-SER-PRO-TYR(NO2)—OH \| |
| | | LISINOPRIL |
| 3 | ACP5 | STREPTOZOTOCIN \| G-CSF \| ANALGESICS \| TPA \| M-CSF |
| 4 | ACTR2 | ETOPOSIDE \| MCP-1 |
| 5 | ACVR1C | GROWTH/DIFFERENTIATION FACTOR-1 \| |
| | | GROWTH/DIFFERENTIATION FACTOR-10 \| ACTIVIN B |
| | | GROWTH/DIFFERENTIATION FACTOR-9 \| |
| | | GROWTH/DIFFERENTIATION FACTOR-7 \| SB-431542 \| |
| | | GROWTH/DIFFERENTIATION FACTOR-3 \| ACTIVIN A \| |
| | | INHIBIN A \| ACTIVIN AB |
| 6 | ADSS | ALANOSINE |
| 7 | AKT1S1 | OSI-027 \| AZD8055 \| MLN0128 |
| 8 | ARID1A | DASATINIB \| Trastuzumab \| GSK126 |
| 9 | ATP6V1A | CRUENTAREN |
| 10 | BAD | AT-101 |
| 11 | BAX | PREDNISOLONE \| METHOTREXATE \| NITROGEN MUSTARD \| |
| | | DOXORUBICIN \| CA4P \| PHENYLEPHRINE \| DHEA \| |
| | | PHENOBARBITAL \| BICALUTAMIDE \| DHT \| MELATONIN \| |
| | | SULFORAPHANE \| MEDROXYPROGESTERONE \| ETOPOSIDE |
| | | \| COBALT \| C5A \| ADM \| FLUDARABINE \| TESTOSTERONE |
| | | UNDECANOATE \| NFX \| QUERCETIN \| M-CSF \| CARVEDILOL |
| | | \| INTERLEUKIN-1 \| VINORELBINE \| SALINE \| ERBITUX \| |
| | | OXALIPLATIN \| ESTRAMUSTINE \| HUMAN CHORIONIC |
| | | GONADOTROPIN \| IL-4 \| PMSG \| NICOTINE \| |
| | | NEUROPROTECTANTS \| TNP-470 \| VERAPAMIL \| RETINOL \| |
| | | ANTHRACYCLINE \| 5-FLUOROURACIL \| BORTEZOMIB \| |
| | | ERYTHROMYCIN \| INHIBINS \| PROCARBAZINE \| |
| | | RAPAMYCIN \| BETA-LAPACHONE \| HYPERBARIC OXYGEN \| |
| | | CIPROFLOXACIN \| BCG \| PRAVASTATIN \| ATENOLOL \| |
| | | INTERFERON ALFA-2B \| OXYTOCIN \| ANTI- |
| | | INFLAMMATORY DRUGS \| LEUCOVORIN \| DOCETAXEL \| |
| | | NALTREXONE \| CAMPTOTHECIN \| PROTEIN PHOSPHATASE |
| | | INHIBITOR \| ANDROSTENEDIONE \| RETINOIC ACID \| |
| | | MITOXANTRONE \| XK469 \| CYCLOPHOSPHAMIDE \| |
| | | DACARBAZINE \| FLT3 LIGAND \| CISPLATIN \| VIOXX \| |
| | | LEVODOPA |
| 12 | BRDT | XD14 \| (+)-JQ1 |
| 13 | CASP5 | M826 \| EMRICASAN \| Z-WEHD-FMK |
| 14 | CATSPER4 | PGF1Î± \| PGE1 \| PGE2 \| PROGESTERONE \| MIBEFRADIL \| |
| | | NNC55-0396 |
| 15 | CDK5 | PURVALANOL B \| BS-194 \| AT-7519 \| ALVOCIDIB \| |
| | | DINACICLIB \| Roniciclib \| ALOISINE A \| RGB-286638 \| |
| | | DINACICLIB \| PURVALANOL A \| CDK1/5 INHIBITOR \| AZD- |
| | | 5438 \| SCH727965 \| AT-7519 \| CDK2 INHIBITOR IV \| 1- |
| | | AZAKENPAULLONE \| GSK-3 INHIBITOR IX \| SELICICLIB \| |
| | | AMINOPURVALANOL A \| DINACICLIB \| TG02 \| DINACICLIB \| |
| | | ALVOCIDIB \| RGB-286638 \| PHA-793887 |
| 16 | CLCN6 | DIDS |
| 17 | CTSL | HORMONES \| LHVS \| KGP94 |
| 18 | E2F3 | LYMPHOTOXIN-Î± \| TUMOUR NECROSIS FACTOR |
| | | MEMBRANE FORM |
| 19 | EEF2 | SS1(DSFV)-PE38 \| DENILEUKIN DIFTITOX \| VB4-845 |
| 20 | EPOR | METHOXY POLYETHYLENE GLYCOL-EPOETIN BETA \| |
| | | DARBEPOETIN ALFA \| HEMATIDE \| HEMATIDE \| |
| | | DARBEPOETIN ALFA \| PEGINESATIDE \| DARBEPOETIN |
| | | ALFA \| DARBEPOETIN ALFA \| ERYTHROPOIETIN \| EPOETIN |
| | | ALFA \| CAPSAICIN \| EPOETIN ALFA \| ERYTHROPOIESIS- |
| | | STIMULATING AGENT \| PEGINESATIDE ACETATE \| LU |
| | | AA24493 \| METHOXY POLYETHYLENE GLYCOL-EPOETIN |
| | | BETA \| PEGINESATIDE \| EPOETIN ALFA \| ERYTHROPOIETIN |
| | | \| EPOETIN ALFA |
| 21 | EREG | PANITUMUMAB \| CETUXIMAB \| HUMAN CHORIONIC |
| | | GONADOTROPIN |
| 22 | EZH1 | GSK343 |
| 23 | F2R | RIGOSERTIB SODIUM \| TYROSINE KINASE INHIBITOR \| |
| | | MORPHINE \| TFLLR-NH2 \| ETHANOL \| ATOPAXAR \| |
| | | VORAPAXAR SULFATE \| WORTMANNIN \| TRANSFORMING |
| | | GROWTH FACTOR \| [3H]HATRAP \| LEPIRUDIN \| |
| | | VORAPAXAR \| PROSTAGLANDIN E1 \| THALIDOMIDE \| |
| | | THROMBIN \| BLEOMYCIN \| RWJ-56110 \| F16357 \| KINASE |
| | | INHIBITOR \| GLUCOCORTICOID \| VORAPAXAR \| |
| | | RUSALATIDE \| ASPIRIN \| DALTEPARIN \| ATROPINE \| ANTI- |
| | | VEGF \| ARGATROBAN \| SCH-530348 \| ATOPAXAR |
| 24 | FGFR4 | BGJ398 \| DOVITINIB \| AZD4547 \| CVBT-141H \| CVBT-141B \| |
| | | ENMD-2076 \| PALIFERMIN \| PALIFERMIN \| BRIVANIB \| ARQ- |
| | | 087 \| FP-1039 \| LY-2874455 \| FGF/VEGF RECEPTOR TYROSINE |
| | | KINASE INHIBITOR, PD173074 \| BGJ-398 \| INFIGRATINIB \| |
| | | ACTB1003 \| LY2874455 \| FGF401 \| ERDAFITINIB \| FGF-6 \| XL- |
| | | 999 \| CP-459632 \| BAY-1163877 \| BRIVANIB ALANINATE \| ISIS- |
| | | FGFR4 \| NINTEDANIB \| DOVITINIB \| ENMD-981693 \| |
| | | TRAFERMIN \| NINTEDANIB ESYLATE \| NINTEDANIB \| AZD- |
| | | 4547 \| BLU9931 \| ORANTINIB |
| 25 | GFER | ETHANOL |
| 26 | GNRH1 | DDS \| LITHIUM \| ABARELIX \| DDC \| GOSERELIN \| GNRH |
| | | PHARMACCINE \| AMINOGLUTETHIMIDE \| |
| | | DIETHYLDITHIOCARBAMATE \| CONJUGATED ESTROGENS |
| | | \| 6-OHDA \| RESERPINE \| VITAMIN E \| LEUPROLIDE \| |
| | | OZARELIX \| TNF-ALPHA \| RALOXIFENE \| CAPTOPRIL \| |
| | | ANTIDEPRESSANT \| BUTYRATE \| BESTATIN \| NORELIN |
| 27 | HDAC9 | SB939 \| PCI-24781 \| TMP269 \| ROMIDEPSIN \| CUDC-101 \| |
| | | VORINOSTAT \| GIVINOSTAT \| CUDC-101 \| BELINOSTAT \| |
| | | BELINOSTAT \| PRACINOSTAT \| AR-42 \| PANOBINOSTAT \| |
| | | PANOBINOSTAT \| PANOBINOSTAT \| PIVANEX \| SCRIPTAID \| |
| | | BELINOSTAT \| MGCD-0103 \| DACINOSTAT \| BELINOSTAT \| |
| | | VORINOSTAT \| TRICHOSTATIN A \| ROMIDEPSIN \| |
| | | VORINOSTAT \| GIVINOSTAT \| CUDC-101 \| GIVINOSTAT \| |
| | | QUISINOSTAT \| ROMIDEPSIN \| ENTINOSTAT \| VALPROIC |
| | | ACID \| PANOBINOSTAT \| RESMINOSTAT \| VORINOSTAT \| |
| | | VALPROIC ACID \| SODIUM PHENYLBUTYRATE |
| 28 | IL12A | BRIAKINUMAB \| AD-RTS-IL-12 \| USTEKINUMAB \| |
| | | VIRULIZIN \| DC-RTS-IL-12 \| BRIAKINUMAB \| CID 446414 \| |
| | | DNA IL-12 \| HUMANIZED SMART ANTI-IL-12 ANTIBODY \| |
| | | Ustekinumab |
| 29 | IL12RB2 | INCELL-1001 + AD-1001 \| AS1409 \| DENILEUKIN DIFTITOX |
| 30 | IL2 | GARLIC EXTRACT \| PHYTOCHEMICALS \| HALOFUGINONE \| |
| | | DIETHYLSTILBESTROL DIPHOSPHATE \| MACROLIDE |
| | | ANTIBIOTIC \| TRICHOSTATIN A \| ALPHA-METHYL-P- |
| | | TYROSINE \| BROMOCRIPTINE \| CSA \| QUININE \| CEFEPIME \| |
| | | CAROTENOIDS \| RCD4 \| HUMAN CHORIONIC |
| | | GONADOTROPIN \| PRAVASTATIN \| PROCHLORPERAZINE \| |
| | | OFLOXACIN \| DHEA \| PENTAMIDINE ISETHIONATE \| |
| | | AMIKACIN SULFATE \| TICARCILLIN \| METOCLOPRAMIDE \| |
| | | LAK \| PEFLOXACIN \| GLYBURIDE \| PROTEASOME |
| | | INHIBITOR \| CALCITRIOL \| ROXITHROMYCIN \| CLOZAPINE \| |
| | | BECLOMETHASONE \| AZIRIDINE \| RTGF-BETA \| |
| | | ITRACONAZOLE \| IMIPENEM \| DIDEMNIN B \| BACILLUS |
| | | CALMETTE-GUERIN \| CELL DIFFERENTIATING AGENT \| |
| | | MONOCLONAL ANTIBODY 17-1A \| MARIJUANA \| |
| | | PARACETAMOL \| PROTEIN KINASE C INHIBITOR \| |
| | | PYRIMETHAMINE \| 4- |
| | | HYDROPEROXYCYCLOPHOSPHAMIDE \| CGP41251 \| |
| | | INTERFERON BETA-1B \| MTX \| CYCLOOXYGENASE |
| | | INHIBITOR \| GARLIC \| SIGNAL TRANSDUCTION |
| | | INHIBITORS \| NG-MONOMETHYL-L-ARGININE \| MISTLETOE |
| | | LECTIN \| GENTAMICIN \| TROPISETRON |
| 31 | IPO7 | MKC-1 |
| 32 | JUN | ALPHA-DIFLUOROMETHYLORNITHINE \| DFMO \| ARSENIC |
| | | TRIOXIDE \| T-5224 \| T-5224 |
| 33 | KCNA4 | VERAPAMIL \| NERISPIRDINE \| DALFAMPRIDINE \| |
| | | TEDISAMIL \| GUANIDINE HYDROCHLORIDE |
| 34 | KCNG2 | TEDISAMIL \| DALFAMPRIDINE \| NERISPIRDINE \| |
| | | GUANIDINE HYDROCHLORIDE |
| 35 | KLK14 | COMPOUND 4D [PMID: 25489658] \| COMPOUND 3 [PMID: |
| | | 23849879] |
| 36 | KRT19 | PD-98059 \| DEXAMETHASONE |
| 37 | LCAT | STREPTOZOTOCIN \| TESTOSTERONE PROPIONATE \| |
| | | TESTOSTERONE \| PREDNISOLONE |
| 38 | LNPEP | COMPOUND 17 [PMID: 23916253] \| INHIBITOR 19 [PMID: |
| | | 21476495] \| TOSEDOSTAT |
| 39 | MAP3K11 | URMC-099 \| CEP-1374 \| CEP-1347 \| COMPOUND 8 [PMID: |
| | | 24044867] |
| 40 | MARK1 | 7-HYDROXYSTAUROSPORINE |
| 41 | MGAM | MIGLITOL \| MIGLITOL \| ACARBOSE \| VOGLIBOSE \| |
| | | MIGLITOL \| VOGLIBOSE \| VOGLIBOSE \| ACARBOSE \| |
| | | ACARBOSE \| MIGLITOL |
| 42 | MMP12 | AZD6605 \| COMPOUND 20 [PMID: 22153340] \| COMPOUND 5 |
| | | [PMID: 24900526] \| V85546 \| ILOMASTAT \| AZD1236 \| |
| | | COMPOUND 1 [PMID: 24900526] \| AZD1236 \| NEOVASTAT \| |
| | | RXP470.1 |
| 43 | OGG1 | CAFFEINE \| SELENIUM |
| 44 | PGD | PENICILLAMINE \| PHENOBARBITAL |
| 45 | PIK3C3 | PKI-587 \| PWT33597 \| XL-765 \| SF1126 \| SAR405 \| GDC-0980 \| |
| | | PX-866 \| GDC-0941 \| COMPOUND 82 [PMID: 21332118] \| |
| | | BKM120 \| BEZ235 \| SF1126 \| XL147 \| PF-4691502 \| VPS34-IN-1 \| |
| | | COMPOUND 28 [PMID: 24387221] \| GSK2636771 \| PI-103 \| |
| | | SOPHORETIN \| BYL719 \| TORIN 2 \| BAY80-6946 \| PIK-III \| |
| | | BGJ398 |
| 46 | PPID | SCY-635 |
| 47 | PRKCA | G√∂ 6983 \| 7-HYDROXYSTAUROSPORINE \| SOPHORETIN \| |
| | | G√∂ 6976 \| BALANOL \| MIDOSTAURIN \| BRYOSTATIN-1 \| |
| | | SOTRASTAURIN \| SOTRASTAURIN \| SOTRASTAURIN |
| | | ACETATE \| CEP-2563 \| RUBOXISTAURIN \| ENZASTAURIN \| |
| | | BRYOSTATIN \| INGENOL MEBUTATE \| AFFINITAC \| |
| | | ENZASTAURIN \| MIDOSTAURIN \| (7S)-HYDROXYL- |
| | | STAUROSPORINE \| INGENOL MEBUTATE \| GSK-690693 |
| 48 | PRMT6 | EPZ020411 \| MS023 \| MS049 \| C21 |
| 49 | PRMT7 | DS-437 |
| 50 | PRMT8 | MS023 \| MS049 |
| 51 | PTK6 | COMPOUND 19A [PMID: 21855335] \| VANDETANIB \| |
| | | TILFRINIB |
| 52 | REGIA | LINOMIDE \| INSULIN |
| 53 | S1PR5 | Siponimod \| FINGOLIMOD HYDROCHLORIDE \| AMISELIMOD |
| | | HYDROCHLORIDE |
| 54 | SERPING1 | C1-INH \| RECOMBINANT HUMAN C1 ESTERASE INHIBITOR |
| 55 | SETBP1 | N/A |
| 56 | SIGMAR1 | E-5842 \| (RS)-PPCC \| RIMCAZOLE \| ANAVEX 1007 \| SSR- |
| | | 125047 \| DEXTROMETHORPHAN \| [3H]PENTAZOCINE \| MS- |
| | | 377 \| ANAVEX 2-73 \| (+)-SKF 10.047 \| NE-100 \| E-6276 \| SIGMA |
| | | ANTAGONIST \| ANAVEX 2-73 \| OPC-14523 \| |
| | | DEXTROMETHORPHAN HYDROBROMIDE MONOHYDRATE |
| | | \| PRX-00023 \| PRE-084 \| RIMCAZOLE \| BD-1047 \| OPC-14523 \| |
| | | SA4503 \| PENTAZOCINE LACTATE \| DEXTROMETHORPHAN |
| | | \| DEXTROMETHORPHAN \| (−)-PENTAZOCINE \| SR-31742A \| |
| | | NALTREXONE \| DEXTROMETHORPHAN POLISTIREX \| |
| | | DEXTROMETHORPHAN \| NE-100 \| PENTAZOCINE |
| | | HYDROCHLORIDE \| DEUTERATED DEXTROMETHORPHAN \| |
| | | ANAVEX 1-41 |
| 57 | SLC29A4 | CIMETIDINE \| DIPYRIDAMOLE \| RHODAMINE123 \| |
| | | DECYNIUM 22 \| QUININE \| FLUOXETINE \| QUINIDINE \| |
| | | VERAPAMIL \| DESIPRAMINE |
| 58 | SLC6A9 | [3H](R)-NPTS \| SSR-103800 \| BITOPERTIN \| GSK931145 \| ORG- |
| | | 24461 \| ALX-5407 \| ORGANON \| [3H]SB-733993 \| [3H]NFPS \| PF- |
| | | 03463275 \| BITOPERTIN \| ORGANON-2 \| [35S]ACPPB \| (R)- |
| | | NFPS \| [3H]N-METHYL-SSR504734 \| LY2365109 \| SSR-504734 \| |
| | | N-METHYL-SSR504734 \| GSK1018921 \| ORGANON-3 \| R1678 \| |
| | | SSR-103800 \| GLIATECH \| [3H]GSK931145 |
| 59 | SLCO2A1 | [3H]PGE2 |
| 60 | SMARCB1 | radiotherapy \| EPZ-6438 \| Alvocidib \| Tazemetostat \| DZNeP \| |
| | | Vorinostat \| PANOBINOSTAT \| Fenretinide \| Palbociclib \| CFI- |
| | | 400945 |
| 61 | SOCS1 | INSULIN \| IL-2 |
| 62 | SRD5A1 | LEVONORGESTREL \| DUTASTERIDE \| FINASTERIDE \| |
| | | FINASTERIDE \| DUTASTERIDE \| DUTASTERIDE |
| 63 | SRD5A3 | DUTASTERIDE |
| 64 | TBXAS1 | NV-52 \| NM-702 \| COMPOUND 7P [PMID: 7861416] \| NV-52 \| |
| | | PMA \| COMPOUND 10A [PMID: 1447738] |
| 65 | TGFB2 | LERDELIMUMAB \| TRABEDERSEN \| BELAGENPUMATUCEL- |
| | | L \| FRESOLIMUMAB \| AP12009 \| FRESOLIMUMAB \| |
| | | LERDELIMUMAB |
| 66 | THBD | SIMVASTATIN \| CILOSTAZOL \| LEVOTHYROXINE \| GINKGO |
| | | BILOBA \| CURCUMIN \| PROSTAGLANDIN E1 |
| 67 | TNFRSF12A | ENAVATUZUMAB \| TWEAK |
| 68 | TPCN1 | VERAPAMIL \| PHOSPHATIDYL (3,5) INOSITOL |
| | | BISPHOSPHATE \| CD2+ |
| 69 | TXK | COMPOUND 31 [PMID: 24915291] \| ACALABRUTINIB \| |
| | | IBRUTINIB \| COMPOUND 38 [PMID: 24915291] \| PRN694 \| |
| | | COMPOUND 23 [PMID: 17600705] \| BOSUTINIB \| COMPOUND |
| | | 7 [PMID: 22464456] |

In one embodiment, the gene or combination is selected from the genes identified in Table IV. Table IV is a list of the top 73 genes identified in the high MOI SARS-CoV-2 screen and the corresponding drugs known to inhibit expression or activity of these genes. These genes are not ranked in the same order as for Table I or Table II. The drugs are listed as inhibiting the corresponding gene (see, e.g., the Drug Gene Interaction database: www.dgidb.org/). Previous reports have established that these small-molecule drugs can inhibit (“mimic loss-of-function”) of the corresponding gene in the list. That is, the drug copies loss-of-function/knock-out of the gene without the need for other means of knocking out gene expression or activity, such as CRISPR, genome editing, etc.

| Rank | Gene | Inhibitor Drug |
|---|---|---|
| 1 | ABCB4 | ORTATAXEL \| AMYLIN \| VINBLASTINE \| AMYLIN \| |
| | | TARIQUIDAR \| TYR0Î±- CGRP (HUMAN) \| VINCRISTINE \| |
| | | PRAMLINTIDE \| CYCLOSPORINE \| MITOMYCIN-C \| Î±- |
| | | CGRP \| ADRENOMEDULLIN \| LY335979 \| |
| | | ADRENOMEDULLIN 2/INTERMEDIN \| CALCITONIN \| |
| | | VALSPODAR \| CURCUMIN I \| [125I]BH-AMY (RAT, |
| | | MOUSE) \| BENZQUINAMIDE \| CBT-1 |
| 2 | ACE2 | COMPOUND 28 [PMID: 18324760] \| XNT [PMID: 18391097] \| |
| | | MLN-4760 \| GL1001 \| ABZ-SER-PRO-TYR(NO2)—OH \| |
| | | LISINOPRIL |
| 3 | ACTB | ETHINYLESTRADIOL \| ENVIRONMENTAL ESTROGEN \| |
| | | CYCLOPHOSPHAMIDE |
| 4 | ACTR2 | ETOPOSIDE \| MCP-1 |
| 5 | ADSS | ALANOSINE |
| 6 | AOX1 | MENADIONE \| ISOVANILLIN |
| 7 | AQP6 | HG2+ |
| 8 | ARID1A | DASATINIB \| Trastuzumab \| GSK126 |
| 9 | ATIC | FOLIC ACID \| METHOTREXATE |
| 10 | ATP6V1A | CRUENTAREN |
| 11 | BAP1 | TRICHOSTATIN A (TSA) \| EVEROLIMUS \| LBH-589 \| |
| | | VALPROIC ACID \| VORINOSTAT (SAHA) \| SUNITINIB |
| 12 | BAX | PREDNISOLONE \| METHOTREXATE \| NITROGEN |
| | | MUSTARD \| DOXORUBICIN \| CA4P \| PHENYLEPHRINE \| |
| | | DHEA \| PHENOBARBITAL \| BICALUTAMIDE \| DHT \| |
| | | MELATONIN \| SULFORAPHANE \| |
| | | MEDROXYPROGESTERONE \| ETOPOSIDE \| COBALT \| C5A |
| | | \| ADM \| FLUDARABINE \| TESTOSTERONE |
| | | UNDECANOATE \| NFX \| QUERCETIN \| M-CSF \| |
| | | CARVEDILOL \| INTERLEUKIN-1 \| VINORELBINE \| SALINE |
| | | \| ERBITUX \| OXALIPLATIN \| ESTRAMUSTINE \| HUMAN |
| | | CHORIONIC GONADOTROPIN \| IL-4 \| PMSG \| NICOTINE \| |
| | | NEUROPROTECTANTS \| TNP-470 \| VERAPAMIL \| RETINOL |
| | | \| ANTHRACYCLINE \| 5-FLUOROURACIL \| BORTEZOMIB \| |
| | | ERYTHROMYCIN \| INHIBINS \| PROCARBAZINE \| |
| | | RAPAMYCIN \| BETA-LAPACHONE \| HYPERBARIC |
| | | OXYGEN \| CIPROFLOXACIN \| BCG \| PRAVASTATIN \| |
| | | ATENOLOL \| INTERFERON ALFA-2B \| OXYTOCIN \| ANTI- |
| | | INFLAMMATORY DRUGS \| LEUCOVORIN \| DOCETAXEL \| |
| | | NALTREXONE \| CAMPTOTHECIN \| PROTEIN |
| | | PHOSPHATASE INHIBITOR \| ANDROSTENEDIONE \| |
| | | RETINOIC ACID \| MITOXANTRONE \| XK469 \| |
| | | CYCLOPHOSPHAMIDE \| DACARBAZINE \| FLT3 LIGAND \| |
| | | CISPLATIN \| VIOXX \| LEVODOPA |
| 13 | BCHE | IMMUNOTOXIN \| RIVASTIGMINE \| ECHOTHIOPHATE |
| | | IODIDE \| ISOFLUROPHATE \| PHYSOSTIGMINE \| |
| | | RIVASTIGMINE \| ISOFLUROPHATE \| DEMECARIUM |
| | | BROMIDE \| ECHOTHIOPHATE IODIDE \| RIVASTIGMINE \| |
| | | HEXAFLURONIUM BROMIDE \| HEXAFLURONIUM |
| | | BROMIDE \| RIVASTIGMINE TARTRATE \| TACRINE |
| | | HYDROCHLORIDE \| PHENOTHIAZINE \| ECHOTHIOPHATE |
| | | IODIDE \| TRIAMCINOLONE \| IRINOTECAN \| |
| | | BAMBUTEROL \| DONEPEZIL \| RIVASTIGMINE \| |
| | | ANESTHETICS \| HEXAFLURONIUM BROMIDE \| TACRINE \| |
| | | Succinylcholine \| PYRIDOSTIGMINE \| DEMECARIUM |
| | | BROMIDE \| BUPIVACAINE \| OPIOIDS |
| 14 | BCL2 | HYDROQUINONE \| PENTOXIFYLLINE \| ENDOTHELIN |
| | | RECEPTOR ANTAGONIST \| VENETOCLAX \| FAS LIGAND \| |
| | | SELENIUM \| ERYTHROMYCIN \| DES \| TAZAROTENE \| |
| | | DOLASTATIN 10 \| MICELLAR PACLITAXEL \| ISIS 3521 \| |
| | | OBATOCLAX \| ABT-263 \| ABT-737 \| CILENGITIDE \| 5- |
| | | FLUORODEOXYURIDINE \| CISPLATIN \| OBLIMERSON \| |
| | | ZD1694 \| URSODEOXYCHOLIC ACID \| CHEMOSENSITIZER |
| | | \| PACLITAXEL \| VALPROIC ACID \| PHENYLBUTYRATE \| |
| | | ORTATAXEL \| ANG1005 \| MITOXANTRONE \| EGFR |
| | | TYROSINE KINASE INHIBITOR \| ALPHA-TOCOPHEROL \| |
| | | INTERLEUKINS \| CISPLATIN \| OBATOCLAX \| |
| | | HYPOXANTHINE \| NSAIDS \| NAVITOCLAX, ABT-263 \| HEN |
| | | EGG LYSOZYME \| DDTC \| NAVITOCLAX \| LIPOSOMAL |
| | | DOXORUBICIN \| AT-101 \| VENETOCLAX \| AT101 \| AT-101 \| |
| | | CARBOPLATIN \| PEG-DOCETAXEL \| GAMMA- |
| | | INTERFERON \| ATRA \| IFN \| TESETAXEL \| OBLIMERSEN \| |
| | | PMSG \| BORTEZOMIB \| BEAUVERICIN \| TGFBETA1 \| |
| | | SAPONIN \| NAVITOCLAX \| MCI-186 \| DOCETAXEL\| |
| | | DOXORUBICIN \| STREPTOZOTOCIN \| C5A \| |
| | | ESTRAMUSTINE \| NAVITOCLAX \| PACLITAXEL \| |
| | | EPIRUBICIN \| 5-AMINOLAEVULINIC ACID \| OBLIMERSEN |
| | | \| CA4P \| ISOTRETINOIN \| ETOPOSIDE \| OBATOCLAX |
| | | MESYLATE \| MILATAXEL \| NDGA \| OXALIPLATIN \| |
| | | VENETOCLAX \| EMD 121974 \| OXALIPLATIN \| PROTEASE |
| | | INHIBITOR \| PACLITAXEL \| HYPERBARIC OXYGEN \| SPC- |
| | | 2996 \| OBLIMERSEN \| METHYLPREDNISOLONE \| ORAL |
| | | CONTRACEPTIVE \| EPISIALIN \| TENIPOSIDE \| TRAIL \| 13- |
| | | CIS-RETINOIC ACID \| RIBAVIRIN |
| 15 | BCORL1 | N/A |
| 16 | BRPF1 | PFI-4 \| COMPOUND 2 [PMID: 25408830] \| COMPOUND 3 |
| | | [PMID: 25408830] \| COMPOUND 34 [PMID: 25974391] \| NI-57 |
| | | \| COMPOUND 1 [PMID: 25408830] \| OF-1 |
| 17 | CA6 | SAR566658 \| ETHOXZOLAMIDE |
| 18 | CDK3 | RGB-286638 \| TG02 \| ALVOCIDIB \| N/A |
| 19 | CHKA | TCD-717 \| DEXAMETHASONE |
| 20 | CHRNG | ROCURONIUM BROMIDE \| RAPACURONIUM BROMIDE \| |
| | | VECURONIUM BROMIDE \| GALLAMINE TRIETHIODIDE \| |
| | | ATRACURIUM BESYLATE \| DOXACURIUM CHLORIDE \| |
| | | METOCURINE IODIDE \| TUBOCURARINE CHLORIDE \| |
| | | MIVACURIUM CHLORIDE \| PIPECURONIUM BROMIDE \| |
| | | SUCCINYLCHOLINE CHLORIDE \| PANCURONIUM |
| | | BROMIDE \| DECAMETHONIUM BROMIDE \| |
| | | CISATRACURIUM BESYLATE |
| 21 | CNTN1 | S-ALLYLGLYCINE |
| 22 | COL11A1 | OCRIPLASMIN \| COLLAGENASE CLOSTRIDIUM |
| | | HISTOLYTICUM |
| 23 | CPS1 | CARGLUMIC ACID \| CARGLUMIC ACID |
| 24 | CPT1B | L-CARNITINE \| PERHEXILINE |
| 25 | CTSL | HORMONES \| LHVS \| KGP94 |
| 26 | DEFA4 | CYTOKINES |
| 27 | DPP9 | 1G244 \| COMPOUND 24DD [PMID: 20684603] |
| 28 | DRD1 | METHYLERGONOVINE MALEATE \| CABERGOLINE \| |
| | | FENOLDOPAM \| PROCHLORPERAZINE \| LISURIDE \| |
| | | OLANZAPINE \| FLUPENTHIXOL \| QUETIAPINE \| |
| | | LOXAPINE \| PERGOLIDE \| ETILEVODOPA \| PERGOLIDE \| |
| | | SCH-23390 \| FENOLDOPAM \| FLUPENTIXOL \| |
| | | OLANZAPINE \| PROMAZINE \| (+)-SKF-82526 \| PERGOLIDE |
| | | MESYLATE \| THIORIDAZINE \| APOMORPHINE \| |
| | | ILOPERIDONE \| HALOPERIDOL \| ZUCLOPENTHIXOL \| |
| | | [3H]SCH-23390 \| MINAPRINE \| PERPHENAZINE \| |
| | | CARPHENAZINE \| CARPHENAZINE \| PROPIOMAZINE \| |
| | | ACETOPHENAZINE \| FENOLDOPAM MESYLATE \| |
| | | PERPHENAZINE \| BSF-78438 \| PROCHLORPERAZINE \| N- |
| | | PROPYLNORAPOMORPHINE \| THIORIDAZINE \| |
| | | PROMAZINE \| CLOZAPINE \| A68930 \| AMOXAPINE \| SKF- |
| | | 76783 \| LEVODOPA \| THIORIDAZINE \| 5- |
| | | HYDROXYTRYPTAMINE \| (+)-ADTN \| NORADRENALINE \| |
| | | FLUPHENAZINE \| A77636 \| PIMOZIDE \| LU AE04621 \| |
| | | FLUPENTHIXOL \| ASENAPINE \| THIETHYLPERAZINE \| |
| | | MELEVODOPA \| METHYLERGONOVINE \| KETANSERIN \| |
| | | FENOLDOPAM \| CLOZAPINE \| XP21279 \| |
| | | CHLORPROTHIXENE \| SPIPERONE \| PROPIOMAZINE \| |
| | | ACETOPHENAZINE \| ZUCLOPENTHIXOL \| LU 02-750 \| |
| | | FLUPHENAZINE \| DOPAMINE \| CHLORPROMAZINE \| SKF- |
| | | 38393 \| BROMOCRIPTINE \| ECOPIPAM \| (−)-SKF-82526 \| |
| | | PERICIAZINE \| METHYLERGONOVINE \| COCAINE \| |
| | | TRIFLUPROMAZINE \| [125I]SCH23982 \| LEVODOPA \| SKF- |
| | | 83566 \| FENOLDOPAM \| FLUPHENAZINE \| |
| | | TRIFLUPROMAZINE \| ROTIGOTINE \| MINAPRINE \| |
| | | ECOPIPAM \| LISURIDE \| THIETHYLPERAZINE \| |
| | | ROTIGOTINE \| SKF-83959 \| ERGOLOID MESYLATES \| |
| | | PERGOLIDE \| CLOZAPINE \| METHYLERGONOVINE \| |
| | | CHLORPROTHIXENE \| D1 AGONIST D2 ANTAGONIST \| |
| | | MESORIDAZINE \| (+)-BUTACLAMOL \| APOMORPHINE \| |
| | | PERGOLIDE \| LISURIDE |
| 29 | DYRK2 | COMPOUND 5B [PMID: 24900464] \| COMPOUND 3B [PMID: |
| | | 23454515] \| LEUCETTINE L41 |
| 30 | ENOSF1 | 3TC \| AZT \| ANTIVIRAL |
| 31 | EREG | PANITUMUMAB \| CETUXIMAB \| HUMAN CHORIONIC |
| | | GONADOTROPIN |
| 32 | ETV1 | TRAMETINIB |
| 33 | FAP | ARI-3099 |
| 34 | FASLG | ANGIOTENSIN CONVERTING ENZYME INHIBITOR \| |
| | | APG101 \| TUMOR NECROSIS FACTOR-BETA |
| 35 | FUS | ATRA |
| 36 | GATA1 | Daunorubicin \| Cytarabine \| N/A |
| 37 | GNAS | CISPLATIN \| 5-FLUOROURACIL |
| 38 | HDAC9 | SB939 \| PCI-24781 \| TMP269 \| ROMIDEPSIN \| CUDC-101 \| |
| | | VORINOSTAT \| GIVINOSTAT \| CUDC-101 \| BELINOSTAT \| |
| | | BELINOSTAT \| PRACINOSTAT \| AR-42 \| PANOBINOSTAT \| |
| | | PANOBINOSTAT \| PANOBINOSTAT \| PIVANEX \| |
| | | SCRIPTAID \| BELINOSTAT \| MGCD-0103 \| DACINOSTAT \| |
| | | BELINOSTAT \| VORINOSTAT \| TRICHOSTATIN A \| |
| | | ROMIDEPSIN \| VORINOSTAT \| GIVINOSTAT \| CUDC-101 \| |
| | | GIVINOSTAT \| QUISINOSTAT \| ROMIDEPSIN \| |
| | | ENTINOSTAT \| VALPROIC ACID \| PANOBINOSTAT \| |
| | | RESMINOSTAT \| VORINOSTAT \| VALPROIC ACID \| |
| | | SODIUM PHENYLBUTYRATE |
| 39 | HLA-DRA | NIVOLUMAB \| PEMBROLIZUMAB \| ATEZOLIZUMAB |
| 40 | HRAS | Pz-1 \| Sirolimus \| Selumetinib \| PD0325901 \| AZD8055 \| EGFR |
| | | INHIBITOR \| BRYOSTATIN-1 \| rigosertib \| VIRAL VECTOR \| |
| | | LONAFARNIB \| REOLYSIN \| Everolimus \| LAK \| |
| | | ADENOVIRAL VECTOR \| STZ \| HYDROXYUREA \| Dasatinib |
| | | \| AZD4547 \| DOCETAXEL \| Binimetinib \| L-744,832 \| BKM120 \| |
| | | EVEROLIMUS \| NS1 \| MK2206 \| IL-6 \| AZ8010 \| Panitumumabm \| |
| | | CI-1040 \| FTI-277 \| BINIMETINIB (MEK162) \| Trametinib \| |
| | | Tipifarnib \| AZD8055 \| E6201 \| GEMCITABINE \| ANTISENSE |
| | | OLIGONUCLEOTIDES \| ANTINEOPLASTIC \| DES \| |
| | | STREPTOZOTOCIN \| DIMETHYL SULFOXIDE \| |
| | | ANTHRACYCLINES \| BYL719 \| INSULIN \| PLX7904 \| |
| | | NITROGLYCERIN \| ERYTHROPOIETIN \| MEK162 \| |
| | | HEXAMETHYLENE BISACETAMIDE \| BEZ235 \| cabozantinib |
| | | \| ISIS-2503 \| BEVACIZUMAB \| ANTIBIOTIC \| SELUMETINIB |
| | | (AZD6244) \| PD173074 \| VITAMIN E \| MNU \| PD-98059 \| |
| | | SF1126 \| Metformin \| Cetuximab \| DIETHYLSTILBESTROL \| |
| | | SCH772984 |
| 41 | HTR1F | 5-CT \| ALMOTRIPTAN MALATE \| Î±-METHYL-5-HT \| GR |
| | | 127935 \| RIZATRIPTAN \| LY344864 \| DIPROPYL-5-CT \| |
| | | NARATRIPTAN \| METHIOTHEPIN \| SUMATRIPTAN \| DOI \| |
| | | TRYPTAMINE \| 5-HYDROXYTRYPTAMINE \| 5-BODMT \| 1- |
| | | NAPHTHYLPIPERAZINE \| DONITRIPTAN \| 5-MEO-DMT \| |
| | | LASMIDITAN \| 2-METHYL-5-HT \| ZOLMITRIPTAN \| |
| | | DIHYDROERGOTAMINE \| NARATRIPTAN \| OLANZAPINE \| |
| | | LASMIDITAN \| LASMIDITAN \| BRL-15572 \| BRL-54443 \| |
| | | RISPERIDONE \| SERTINDOLE \| XANOMELINE \| |
| | | METERGOLINE \| METHYLERGONOVINE \| |
| | | ZOLMITRIPTAN \| 8-OH-DPAT \| QUETIAPINE \| 5-MEOT \| |
| | | TFMPP \| ELETRIPTAN HYDROBROMIDE \| NAN 190 \| |
| | | ZOLMITRIPTAN \| ELETRIPTAN \| [3H]LY334370 \| |
| | | SUMATRIPTAN \| LY334370 \| METHYSERGIDE \| |
| | | NARATRIPTAN \| YOHIMBINE \| ERGOTAMINE \| |
| | | SUMATRIPTAN \| CLOZAPINE |
| 42 | HYAL2 | EPINEPHRINE \| HORMONES \| DEXAMETHASONE |
| 43 | IL23A | BRIAKINUMAB \| BRIAKINUMAB \| CNTO 1275 \| |
| | | USTEKINUMAB \| Ustekinumab \| USTEKINUMAB \| |
| | | USTEKINUMAB \| TILDRAKIZUMAB \| USTEKINUMAB \| |
| | | GUSELKUMAB |
| 44 | ITGA4 | VEDOLIZUMAB \| VEDOLIZUMAB \| ATL/TV1102 \| |
| | | NATALIZUMAB \| FIRATEGRAST \| CDP323 \| ATL1102 \| |
| | | VEDOLIZMAB \| NATALIZUMAB \| FIRATEGRAST \| |
| | | GSK683699 \| SB-683699 \| R1295 \| FIRATEGRAST \| |
| | | NATALIZUMAB \| R1295 \| NATALIZUMAB |
| 45 | KDM4E | ML324 \| COMPOUND 6A [PMID: 18942826] |
| 46 | KRT18 | ANTIVIRAL \| MITOMYCIN-C \| IL-6 \| RIBAVIRIN |
| 47 | LY96 | L6H21 \| ERITORAN TETRASODIUM \| NEOCEPTIN-3 |
| 48 | MAP3K10 | CEP-1374 \| URMC-099 \| CEP-1347 |
| 49 | MAP3K3 | COMPOUND 5N [PMID: 20483621] |
| 50 | MMP9 | ANDECALIXIMAB \| MARIMASTAT \| PG-116800 \| |
| | | NEOVASTAT \| COMPOUND 29E [PMID: 23631440] \| CGS- |
| | | 27023A \| PRINOMASTAT \| ANDECALIXIMAB \| S-3304 \| |
| | | BEVACIZUMAB \| SB-3CT \| ILOMASTAT \| COL-3 \| AZD6605 |
| | | \| AZD1236 \| SL422 \| TST10088 \| MARIMASTAT \| AE-941 \| |
| | | COMPOUND 1A [PMID: 15055993] |
| 51 | MYB | RETINOID \| 12-O-TETRADECANOYLPHORBOL-13- |
| | | ACETATE \| LR3001 \| 5-FLUOROURACIL \| DOXORUBICIN \| |
| | | G4460 \| TAXOL |
| 52 | NDUFB2 | NV-128 \| ME-344 \| METFORMIN HYDROCHLORIDE |
| 53 | NDUFV1 | NV-128 \| ME-344 \| METFORMIN HYDROCHLORIDE |
| 54 | OSM | IL-10 \| TNF-ALPHA |
| 55 | PDE6A | COMPOUND 53 [PMID: 19631533] \| PENTOXIFYLLINE \| |
| | | DIPYRIDAMOLE |
| 56 | PGD | PENICILLAMINE \| PHENOBARBITAL |
| 57 | PIK3R4 | PI-103 \| BYL719 \| BYL719 \| BUPARLISIB HYDROCHLORIDE |
| | | \| PX-866 \| GDC-0941 \| PKI-587 \| BEZ235 \| GSK2126458 \| |
| | | SF1126 \| PF-4691502 \| PWT33597 \| PKI-587 \| GDC-0980 \| XL- |
| | | 765 \| BAY80-6946 \| BAY80-6946 \| PX-866 \| BGJ398 \| |
| | | GSK2636771 \| XL147 \| BKM120 \| SOPHORETIN \| |
| | | DACTOLISIB |
| 58 | POLE | FLUDARABINE PHOSPHATE \| PEMBROLIZUMAB \| |
| | | GEMCITABINE HYDROCHLORIDE \| CLOFARABINE \| |
| | | CYTARABINE |
| 59 | PSMA3 | CARFILZOMIB \| BORTEZOMIB \| OPROZOMIB \| IXAZOMIB |
| | | CITRATE \| MARIZOMIB \| BORTEZOMIB \| CARFILZOMIB |
| 60 | PSMB4 | OPROZOMIB \| CARFILZOMIB \| BORTEZOMIB \| IXAZOMIB |
| | | CITRATE \| MARIZOMIB \| BORTEZOMIB \| CARFILZOMIB |
| 61 | PSMB9 | CARFILZOMIB \| OPROZOMIB \| BORTEZOMIB \| |
| | | BORTEZOMIB \| MARIZOMIB \| IXAZOMIB CITRATE \| |
| | | CARFILZOMIB |
| 62 | PSMC4 | CARFILZOMIB \| OPROZOMIB \| BORTEZOMIB \| IXAZOMIB |
| | | CITRATE \| CARFILZOMIB \| BORTEZOMIB |
| 63 | SIAH1 | OMIGAPIL |
| 64 | SLC18A2 | LISDEXAMFETAMINE DIMESYLATE \| [3H]TBZOH \| |
| | | TETRABENAZINE \| VALBENAZINE \| |
| | | DEXTROAMPHETAMINE \| RESERPINE \| TETRABENAZINE |
| | | \| BENZPHETAMINE HYDROCHLORIDE \| LOBELINE \| |
| | | RESERPINE \| [125I]IODOVINYL-TBZ \| ALSEROXYLON \| |
| | | DEXTROAMPHETAMINE SULFATE \| TETRABENAZINE \| |
| | | RESERPINE \| DEUTETRABENAZINE \| RESERPINE \| |
| | | RESCINNAMINE \| KETANSERIN \| METHAMPHETAMINE \| |
| | | DEXTROAMPHETAMINE \| DESERPIDINE \| |
| | | TETRABENAZINE \| DEXTROAMPHETAMINE ADIPATE \| |
| | | NBI-98854 \| ALSEROXYLON \| DEXTROAMPHETAMINE |
| | | SACCHARATE \| RESERPINE \| ALSEROXYLON \| [11C]DTBZ |
| | | \| NRP104 \| AMPHETAMINE \| [1251]7-AZIDO-8- |
| | | IODOKETANSERINE \| TETRABENAZINE |
| 65 | SLCO1A2 | TESTOSTERONE \| [3H]ESTRONE-3-SULPHATE \| |
| | | RIFAMYCIN SV \| HORMONES \| RIFAMPICIN \| NARINGIN \| |
| | | [3H]BSP \| [3H]DPDPE |
| 66 | SLCO1B1 | RIFAMYCIN SV \| CLOFIBRATE \| GEMFIBROZIL \| |
| | | GEMFIBROZIL \| FENOFIBRATE \| ESTRONE-3-SULPHATE \| |
| | | GLYCYRRHIZIN \| METHOTREXATE \| TROGLITAZONE \| |
| | | DOCETAXEL \| RIFAMPICIN \| INDOCY ANINE GREEN \| |
| | | PRAVASTATIN \| Rosuvastatin \| [3H]ESTRADIOL-17Î²- |
| | | GLUCURONIDE \| PRAVASTATIN \| [3H]ESTRONE-3- |
| | | SULPHATE \| CYCLOSPORIN A \| ATORVASTATIN |
| 67 | SMAD4 | Sapanisertib \| CETUXIMAB \| Gemcitabine \| 5- |
| | | FLUOROURACIL \| Cisplatin \| Crizotinib \| N/A \| Paclitaxel \| |
| | | Carboplatin \| SD-093 \| TGF \| Irinotecan \| Imatinib \| Alectinib |
| 68 | SMYD2 | LLY-507 \| AZ505 \| BAY-598 |
| 69 | SOAT1 | NIFEDIPINE \| 6-HYDROXYDOPAMINE \| |
| | | DEXAMETHASONE \| TESTOSTERONE \| EMZETIBE \| |
| | | ATORVASTATIN \| URSODEOXYCHOLIC ACID \| |
| | | LOVASTATIN |
| 70 | TGFBR2 | COMPOUND 15B [PMID: 16539403] \| COMPOUND 13A |
| | | [PMID: 23639540] \| TG-C \| LY2109761 \| COMPOUND 13D |
| | | [PMID: 23639540] |
| 71 | TGFBR3 | CYTOKINES \| DEXAMETHASONE \| RANTES \| BFGF \| GM- |
| | | CSF \| TG-C \| TGF-BETA 2 \| RETINOIC ACID |
| 72 | TOP1 | TOPOTECAN \| LIPOTECAN \| IRINOTECAN \| IRINOTECAN \| |
| | | BECATECARIN \| CAFFEINE \| ETIRINOTECAN PEGOL \| |
| | | IRINOTECAN HYDROCHLORIDE HYDRATE \| (S)-DRF-1042 |
| | | \| CAMPTOTHECIN \| IRINOTECAN \| ARQ-501 \| IRINOTECAN |
| | | SUCROSOFATE \| LE-SN38 \| IRINOTECAN \| PEG- |
| | | CAMPTOTHECIN \| IRINOTECAN \| PEG-IRINOTECAN \| |
| | | TOPOTECAN \| TOPOTECAN \| LURTOTECAN \| |
| | | TOPOISOMERASE INHIBITORS \| NKTR-102 \| RUBITECAN \| |
| | | KARENITECIN \| IRINOTECAN \| NEW AGENTS \| CISPLATIN |
| | | \| CYCLOPHOSPHAMIDE \| C-1311 \| TOPOTECAN \| |
| | | CAMPTOTHECIN \| PEG-SN38 \| N/A |
| 73 | TPBG | NAPTUMOMAB ESTAFENATOX \| ANATUMOMAB |
| | | MAFENATOX |

The following Table V identifies certain available inhibitors, target genes and notes the SelleckChem or MedChem Express vendor numbers for the inhibitors.

TABLE V

| | Available | SelleckChem/MedChem Express |
|---|---|---|
| Target Gene | inhibitors | Vendor Code |
| HDAC9 | Vorinostat | S1047 |
| | Belinostat | S1085 |
| | Panobinostat | S1030 |
| | Pracinostat | S1515 |
| | BRD 4354 | BRD4354 |
| | TMP195 | S8502 |
| Cathepsin L | Odanacatib | S1115 |
| | MG-101 (ALLN) | S7386 |
| | SID 26681509 | SID2668 |
| PRKCA | Tamoxifen | S1238 |
| | Sotrastaurin | S2791 |
| | Staurosporine | S1421 |
| | Enzastaurin | S1055 |
| | Serabelisib (MLN1117) | S8581 |
| PIK3C3 | Idelalisib | S2226 |
| (VPS34) | Buparlisib | S2247 |
| | SAR405 | S7682 |
| | VVPS34 inhibitor 1 | S8456 |
| | (Compound 19) | |
| | Vps34-PIK-III | S7683 |
| | Autophinib | S8596 |
| CALR | Gentamicin | S4030 |
| DRD2 | Quetiapine | S5741 |
| | Olanzapine | S2493 |
| MMP12 | Ilomastat | S7157 |
| | Doxycycline Hyclate | S4163 |
| MAPK3 | PD0325901(Mirdametinib) | S1036 |
| | Binimetinib (MEK162) | S7007 |
| BRPF1 | GSK6853 | GSK6853 |
| control | Remdesivir | S8932 |

In one embodiment, the gene is PIK3C3. In another embodiment, the gene or gene combination is selected from RAB7A, ACE2, ACTR2, ACTR3, ARPC3, and ARPC4. In another embodiment, the gene or combination comprises one or more of CTSL, ATP6AP1, ATP6AP2, ATP6V0B ATP6V0C, ATP6V0D1, ATP6V1A, ATP6V1B2, ATP6V1C1, ATP6V1E1, ATP6V1G1, ATP6V1H, TMEM199, and TOR1AIP1. In another embodiment, the gene or combination comprises one or more of VPS26A, VPS29, VPS35, and SNX27. In another embodiment, the gene or combination comprises one or more of COMMD2, COMMD3, COMMD3-BMI1, and COMMD4. In still a further embodiment, the gene or combination comprises one or more of PIK3C3/VPS34, WDR81, and ACP5.

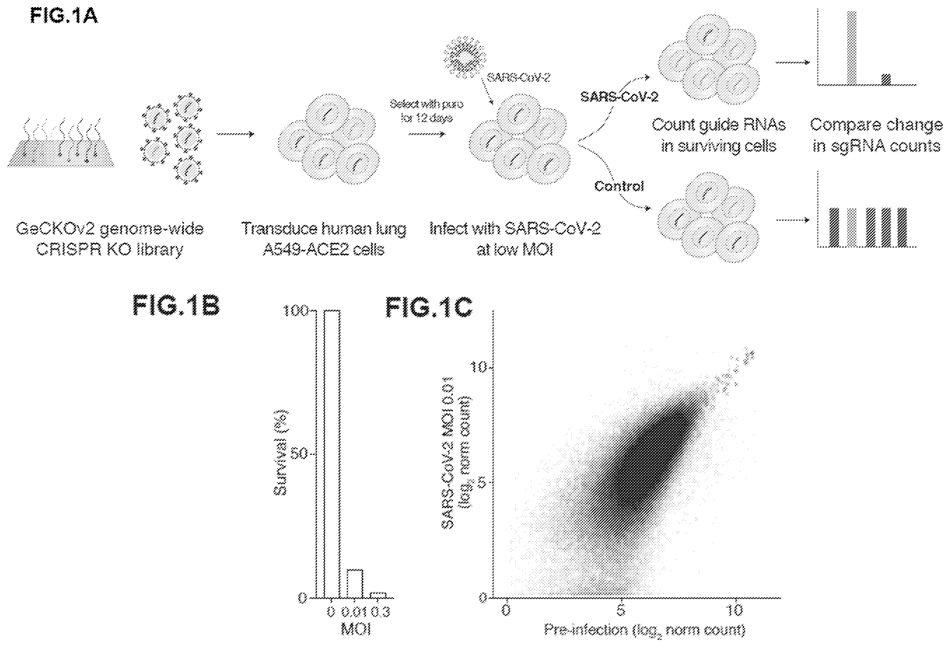

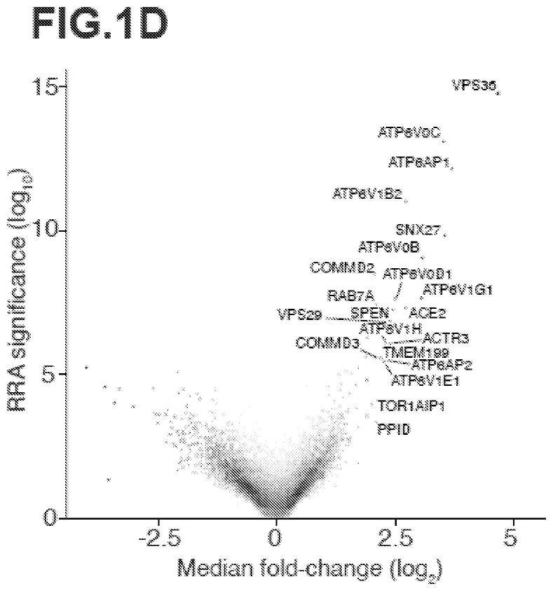

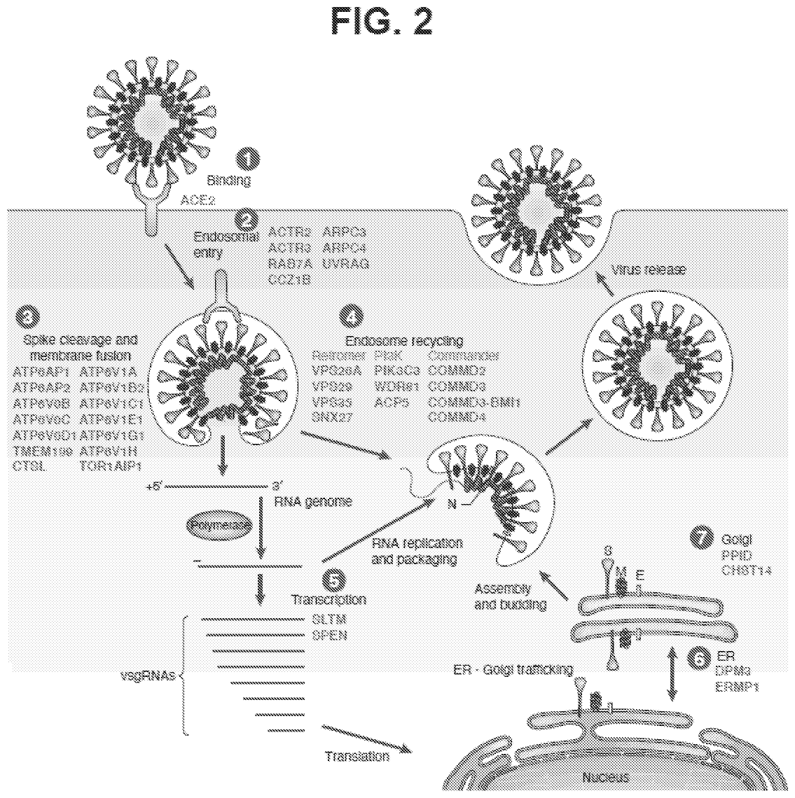

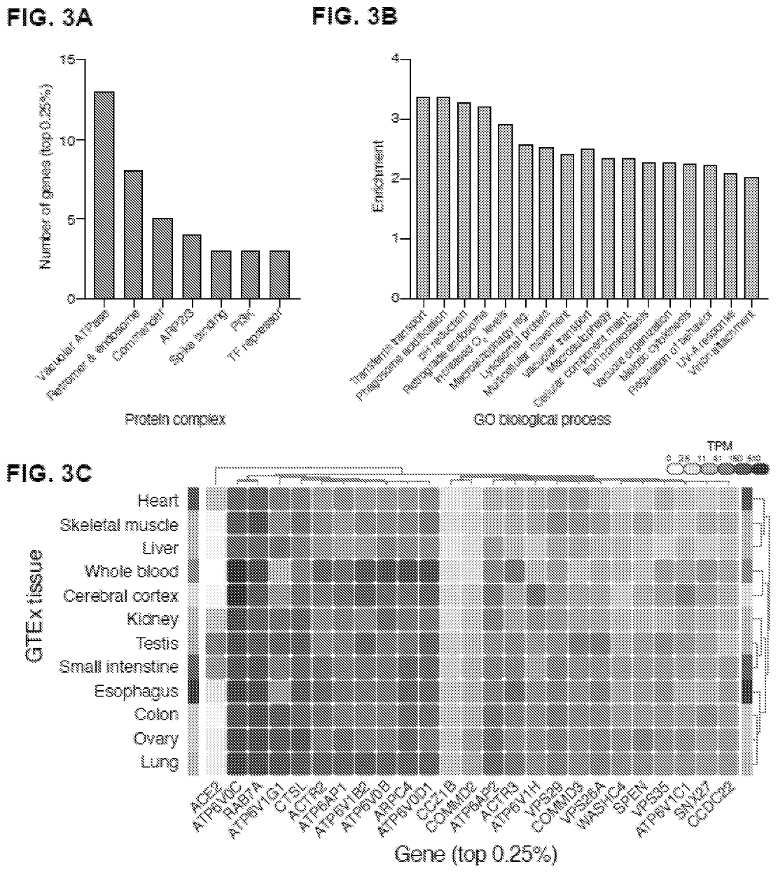

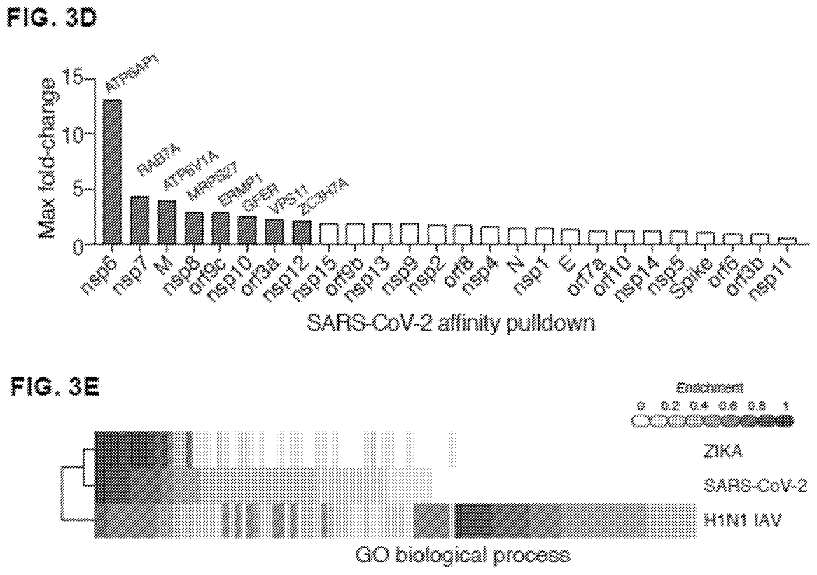

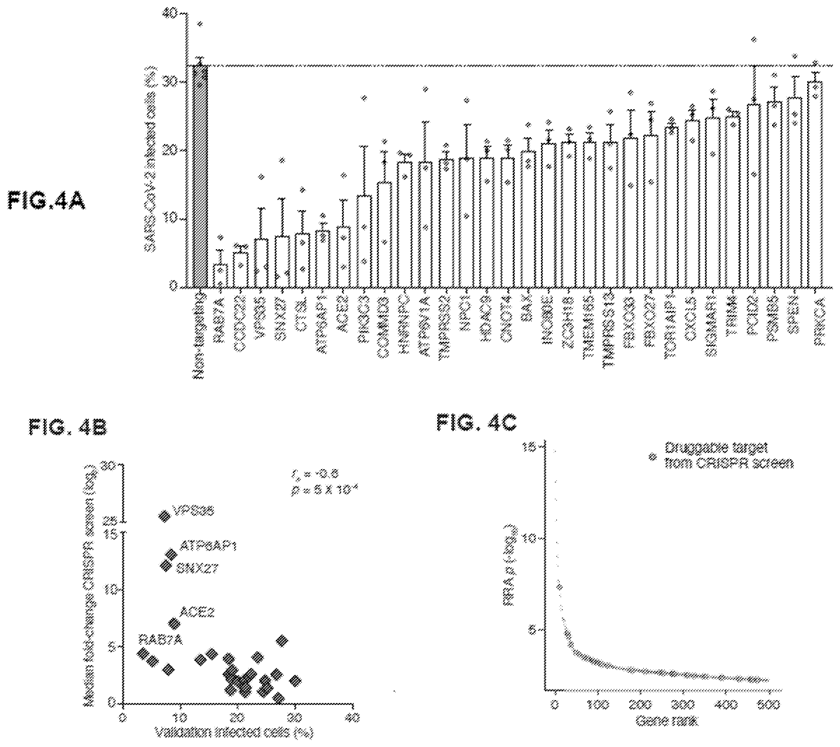

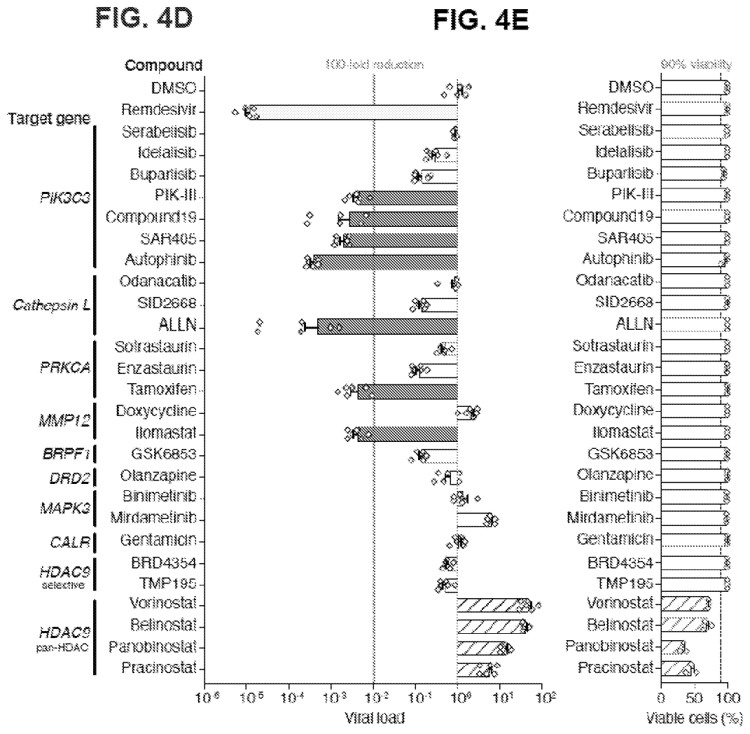

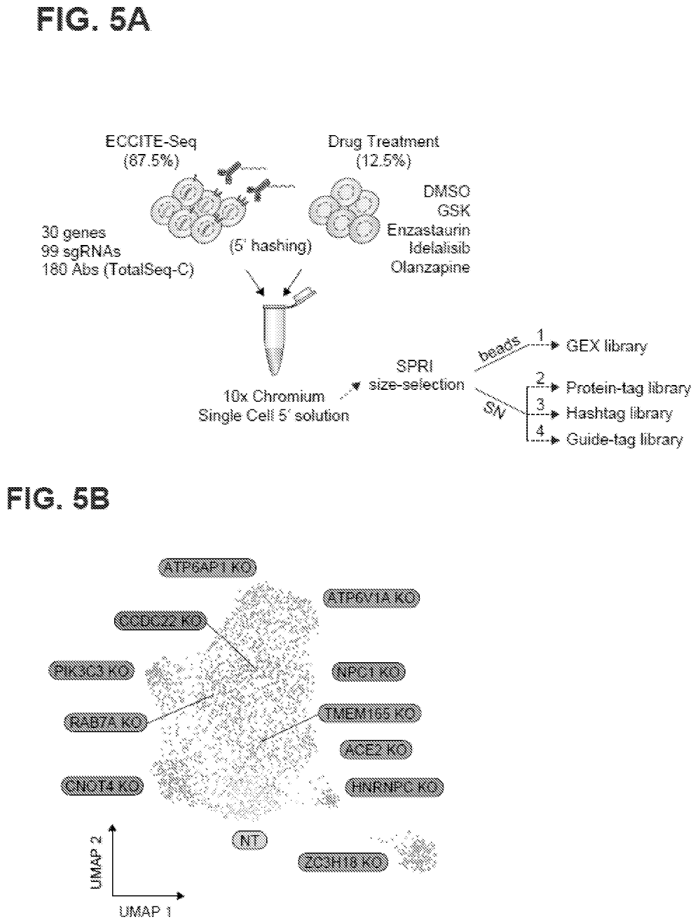

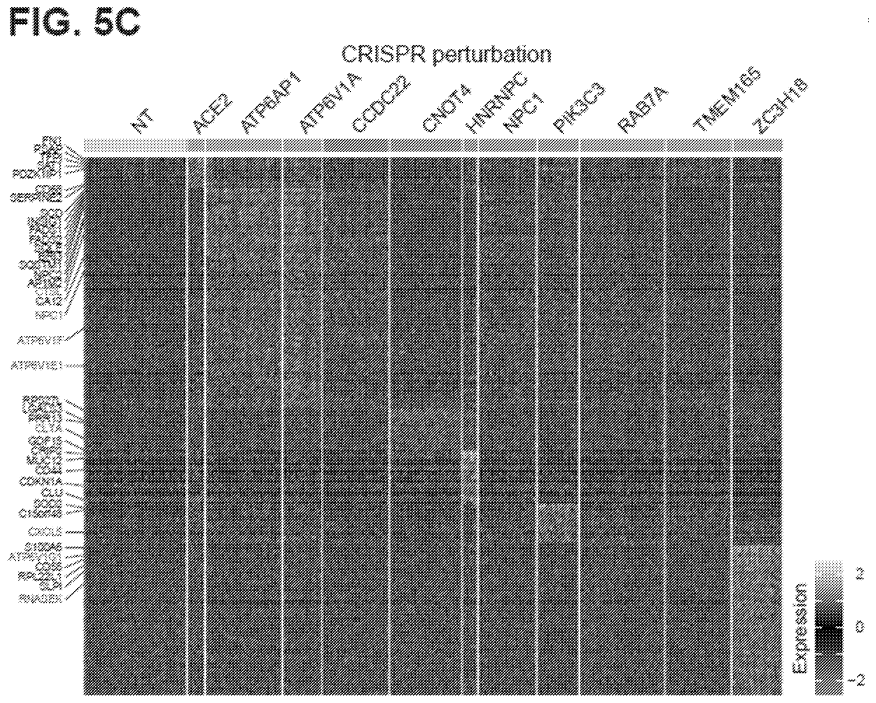

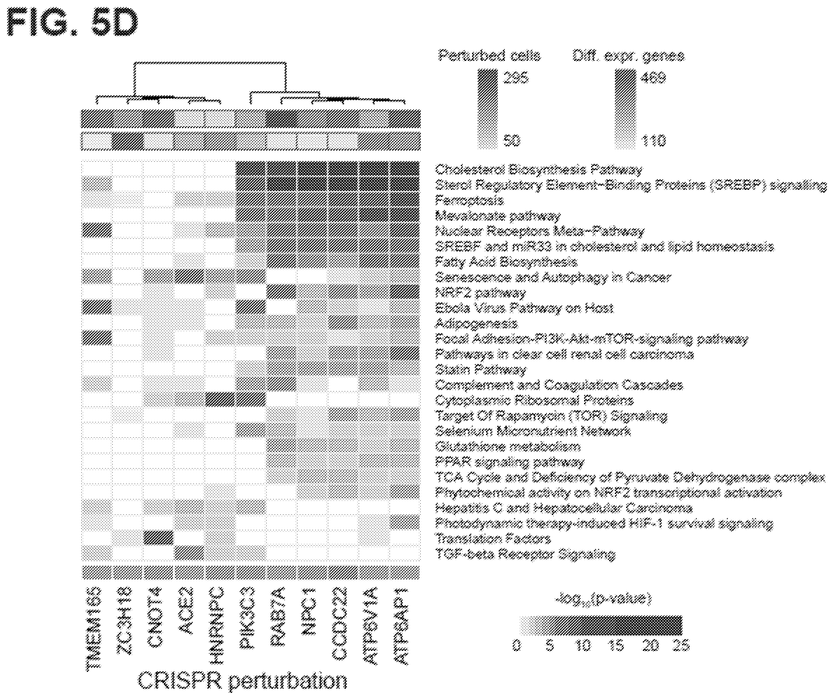

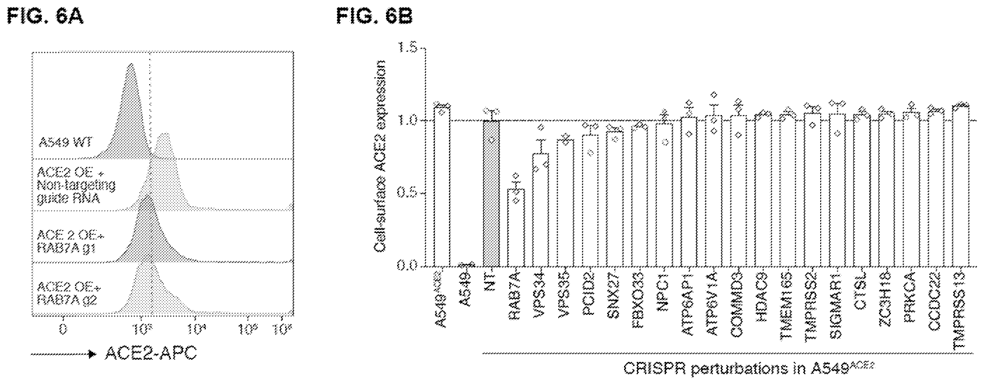

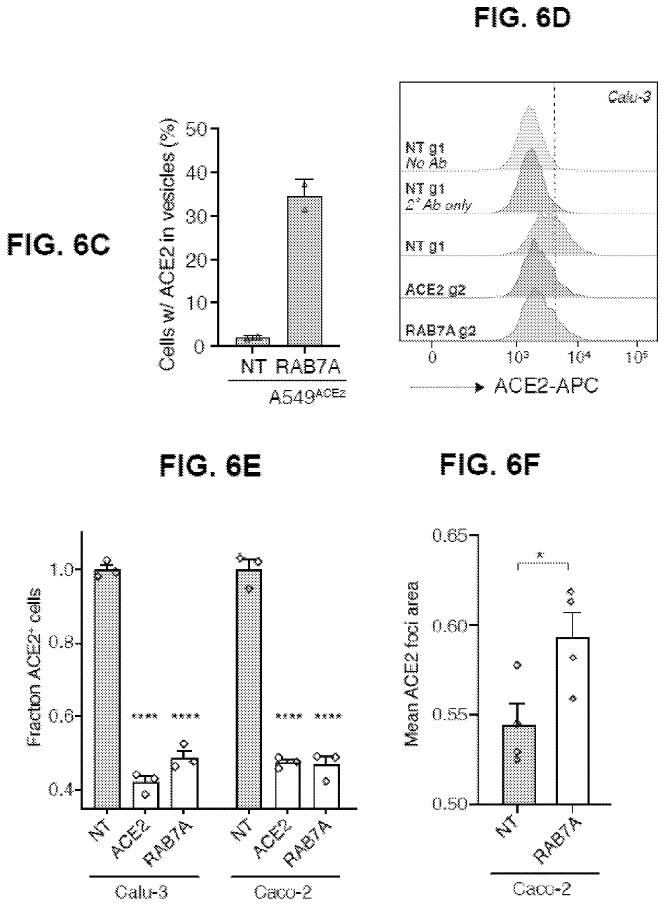

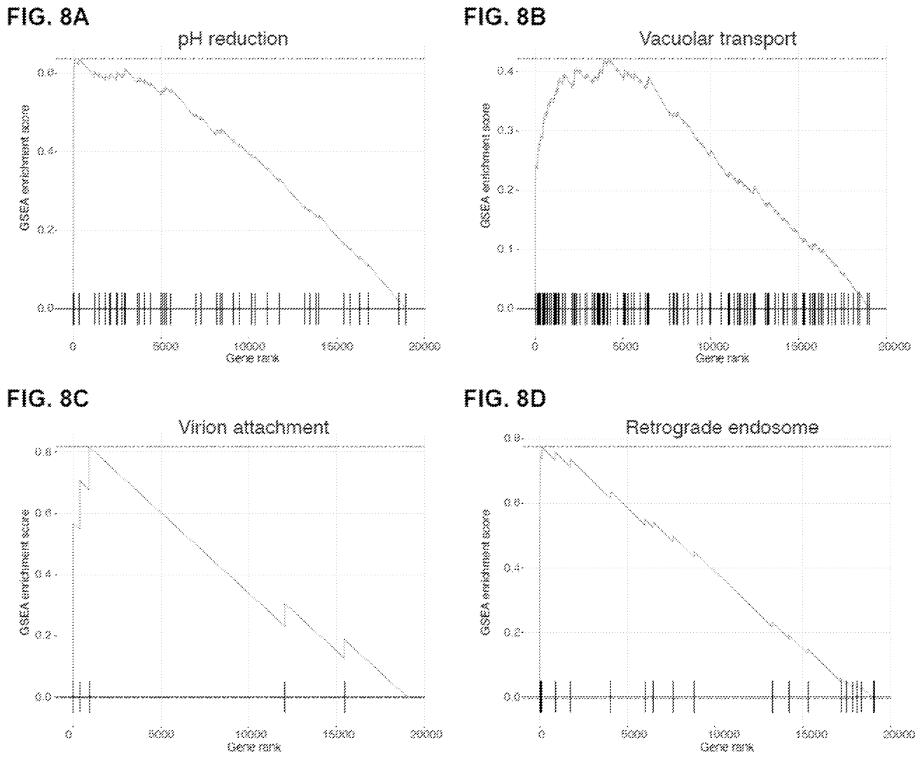

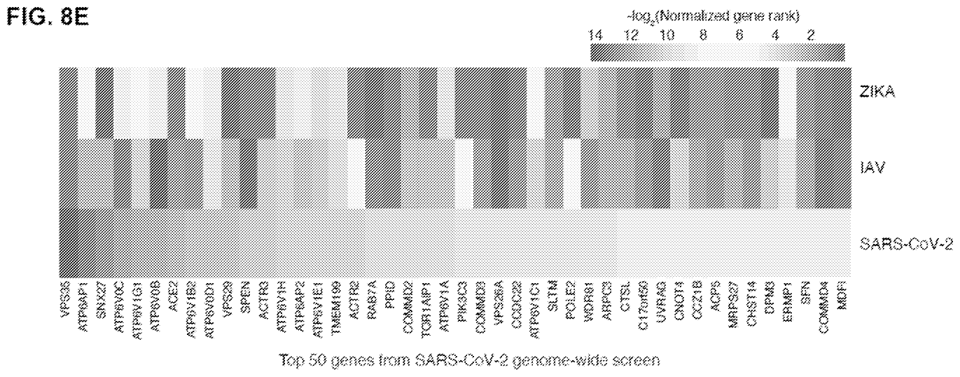

In yet another embodiment, the gene or combination comprises one or more of DPM3, ERMP1, PPID, CHST14, SLTM and SPEN. The various gene combinations can also include one or more of PIK3C3, Cathepsin L, PRKCA, MMP12, BRPF1, DRD2, MAPK3, CALR and HCAC9. Still other combinations or selections of genes are shown in FIG. 1D, in FIG. 2, in FIG. 3C, in FIG. 4A or 4D, in FIG. 5C, in FIG. 6B, in FIG. 8E. In still another embodiment, the genes include one or more of the 69 genes identified in Table III. In yet another embodiment, the genes to be inhibited are one or more of the 73 genes identified in Table IV. Still other combination of genes can be inhibited in the practice of the methods, therapeutic regimens or compositions described herein.

By the term “druggable gene” as used herein is meant a human gene for which an inhibitor of expression, activity or signalling is known and identified. For example, see the 69 low MOI druggable genes and inhibitors in Table III and the 73 high MOI druggable genes and corresponding inhibitors in Table IV. In one embodiment as mentioned above, the druggable gene is PIK3C3.

### C. Inhibitor, Blocker, Antagonist

By the general terms “blocker”, “inhibitor” or “antagonist” is meant an agent that inhibits, either partially or fully, the activity or production of a target molecule, e.g., as used herein, specifically one or more of one or a combination of the human subject's genes required for viral infection. In one embodiment, the viral infection is caused by SARS-CoV-2. In particular, these terms refer to a composition or compound or agent capable of decreasing levels of gene expression, mRNA levels, protein levels or protein activity of the target molecule. Illustrative forms of antagonists include, for example, proteins, polypeptides, peptides (such as cyclic peptides), antibodies or antibody fragments, peptide mimetics, nucleic acid molecules, antisense molecules, ribozymes, aptamers, RNAi molecules, and small organic molecules. Illustrative non-limiting mechanisms of antagonist inhibition include repression of ligand synthesis and/or stability (e.g., using, antisense, ribozymes or RNAi compositions targeting the ligand gene/nucleic acid), blocking of binding of the ligand to its cognate receptor (e.g., using anti-ligand aptamers, antibodies or a soluble, decoy cognate receptor), repression of receptor synthesis and/or stability (e.g., using, antisense, ribozymes or RNAi compositions targeting the ligand receptor gene/nucleic acid), blocking of the binding of the receptor to its cognate receptor (e.g., using receptor antibodies) and blocking of the activation of the receptor by its cognate ligand (e.g., using receptor tyrosine kinase inhibitors). In addition, the blocker or inhibitor may directly or indirectly inhibit the target molecule.

In one embodiment, the inhibitor(s) are selected from one or a combination of the inhibitors identified in Table III or Table IV. In another embodiment, the inhibitor or a combination of inhibitors is identified in FIG. 4D or 4E. In another embodiment, the inhibitor useful in the methods is one or a combination a combination of Remdesivir, PIK-III, Compound 19, SAR405, Autophinib, ALLN, Tamoxifen and Ilomastat. The selected inhibitor is administered to the subject pre-infection or post-infection at a dosage effective to mimic a loss of function of its corresponding gene.

i. Salts—The compositions described herein also includes salts of the specific compounds described in Tables III or IV. As used herein, “salts” refers to derivatives of the disclosed compounds wherein the parent compound is modified by converting an existing acid or base moiety to its salt form. Examples of salts include, but are not limited to, mineral acid (such as HCl, HBr, H₂SO₄) or organic acid (such as acetic acid, benzoic acid, trifluoroacetic acid) salts of basic residues such as amines; alkali (such as Li, Na, K, Mg, Ca) or organic (such as trialkyl ammonium) salts of acidic residues such as carboxylic acids; and the like. The salts of compounds described or referenced herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile (ACN) are preferred.

The “pharmaceutically acceptable salts” of compounds described herein or incorporated by reference include a subset of the “salts” described above which are, conventional non-toxic salts of the parent compound formed, for example, from non-toxic inorganic or organic acids. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

ii. Prodrug—By the term “prodrug” is meant a compound or molecule or agent that, after administration, is metabolized (i.e., converted within the body) into the parent pharmacologically active molecule or compound, e.g., inhibitor of one or more of the gene identified herein. Prodrugs are substantially, if not completely, in a pharmacologically inactive form that is converted or metabolized to an active form (i.e., drug)—such as within the body or cells, typically by the action of, for example, endogenous enzymes or other chemicals and/or conditions. Instead of administering an active molecule directly, a corresponding prodrug is used to improve how the composition/active molecule is absorbed, distributed, metabolized, and excreted. Prodrugs are often designed to improve bioavailability or how selectively the drug interacts with cells or processes that are not its intended target. This reduces adverse or unintended undesirable or severe side effects of the active molecule or drug.

iii. Biosimilar—A “biosimilar” is a biological product, generally a large and complex molecule, produced from living organisms, and monitored to ensure consistent quality that is highly similar to a reference product, e.g., an already FDA-approved biological drug. A biosimilar that receives FDA approval must have no clinically meaningful differences from the reference drug in purity, safety, molecular structure and bioactivity, or potency.

iv. Antibody and Fragments—By the term “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

The antibody may be a naturally occurring antibody or may be a synthetic or modified antibody (e.g., a recombinantly generated antibody; a chimeric antibody; a bispecific antibody; a humanized antibody; a camelid antibody; and the like). The antibody may comprise at least one purification tag. In a particular embodiment, the framework antibody is an antibody fragment. The term “antibody fragment” includes a portion of an antibody that is an antigen binding fragment or single chains thereof. An antibody fragment can be a synthetically or genetically engineered polypeptide. Examples of binding fragments encompassed within the term “antigen-binding portion” of an antibody include (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding fragment” of an antibody. These antibody fragments are obtained using conventional techniques known to those in the art, and the fragments can be screened for utility in the same manner as whole antibodies. Antibody fragments include, without limitation, immunoglobulin fragments including, without limitation: single domain (Dab; e.g., single variable light or heavy chain domain), Fab, Fab′, F(ab′)2, and F(v); and fusions (e.g., via a linker) of these immunoglobulin fragments including, without limitation: scFv, scFv2, scFv-Fc, minibody, diabody, triabody, and tetrabody. The antibody may also be a protein (e.g., a fusion protein) comprising at least one antibody or antibody fragment.

The antibodies useful in the methods are preferably “immunologically specific”, which refers to proteins/polypeptides, particularly antibodies, that bind to one or more epitopes of a protein or compound of interest, but which do not substantially recognize and bind other molecules in a sample containing a mixed population of antigenic biological molecules.

The antibodies useful in inhibiting the genes or gene products of the human genes identified herein may be further modified. For example, the antibodies may be humanized. In a particular embodiment, the antibodies (or a portion thereof) are inserted into the backbone of an antibody or antibody fragment construct. For example, the variable light domain and/or variable heavy domain of the antibodies of the instant invention may be inserted into another antibody construct. Methods for recombinantly producing antibodies are well-known in the art. Indeed, commercial vectors for certain antibody and antibody fragment constructs are available.

The antibodies of the instant invention may also be conjugated/linked to other components. For example, the antibodies may be operably linked (e.g., covalently linked, optionally, through a linker) to at least one cell penetrating peptide, detectable agent, imaging agent, or contrast agent. The antibodies of the instant invention may also comprise at least one purification tag (e.g., a His-tag). In a particular embodiment, the antibody is conjugated to a cell penetrating peptide.

V. Aptamer—The term “aptamer” refers to a peptide or nucleic acid that has an inhibitory effect on a target. Inhibition of the target by the aptamer can occur by binding of the target, by catalytically altering the target, by reacting with the target in a way which modifies the target or the functional activity of the target, by ionically or covalently attaching to the target as in a suicide inhibitor or by facilitating the reaction between the target and another molecule. Aptamers can be peptides, ribonucleotides, deoxyribonucleotides, other nucleic acids or a mixture of the different types of nucleic acids. Aptamers can comprise one or more modified amino acid, bases, sugars, polyethylene glycol spacers or phosphate backbone units as described in further detail herein.

vi. RNA and DNA—The terms “RNA interference,” “RNAi,” “miRNA,” and “siRNA” refer to any method by which expression of a gene or gene product is decreased by introducing into a target cell one or more double-stranded RNAs, which are homologous to a gene of interest (particularly to the messenger RNA of the gene of interest). Gene therapy, i.e., the manipulation of RNA or DNA using recombinant technology and/or treating or preventing viral disease by introducing modified RNA or modified DNA into cells via a number of widely known and experimental vectors, recombinant viruses and CRISPR technologies, may also be employed in delivering, via modified RNA or modified DNA, effective inhibition of the pathways and gene products of the host genes identified herein as necessary to viral infection to accomplish the outcomes described herein with the combination therapies described. Such genetic manipulation can also employ gene editing techniques such as CRISPR (Clustered Regularly Interspaced Short Palindromic Repeats) and TALEN (transcription activator-like effector genome modification), among others. See, for example, the textbook National Academies of Sciences, Engineering, and Medicine. 2017. Human Genome Editing: Science, Ethics, and Governance. Washington, DC: The National Academies Press. https://doi.org/10.17226/24623, incorporated by reference herein for details of such methods.

vii. Small Molecule—The term “small molecule” when applied to a pharmaceutical generally refers to a non-biologic, organic compound that affects a biologic process which has a relatively low molecular weight, below approximately 900 daltons. Small molecule drugs have an easily identifiable structure, that can be replicated synthetically with high confidence. In one embodiment a small molecule has a molecular weight below 550 daltons to increase the probability that the molecule is compatible with the human digestive system's intracellular absorption ability. Small molecule drugs are normally administered orally, as tablets. The term small molecule drug is used to contrast them with biologic drugs, which are relatively large molecules, such as peptides, proteins and recombinant protein fusions, frequently produced using a living organism. Among the compositions identified in Tables III or IV are a host of suitable small molecules.

### D. Effective Amounts, Routes

A “therapeutically effective amount” of a compound or a pharmaceutical composition refers to an amount effective to prevent, inhibit, treat, or lessen the symptoms of a particular viral disorder or disease. For example, “therapeutically effective amount” may refer to an amount sufficient to reduce the typical symptoms of fever, cough, inflammation, loss of smell, loss of breath and cytokine “storm” effects of SARS-CoV-2 infection, among other symptoms. Other viral diseases that may be treated similarly may be related ZIKA or MERS or other coronavirus infections.

A “pharmaceutically acceptable excipient or carrier” refers to, without limitation, a diluent, adjuvant, excipient, auxiliary agent or vehicle with which an active agent of the present invention is administered. Pharmaceutically acceptable carriers are those approved by a regulatory agency of the Federal or a state government or listed in the U.S. Pharmacopeia or other generally recognized pharmacopeia for use in animals, and more particularly in humans, can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “treatment” refers to any method used that imparts a benefit to the subject, i.e., which can alleviate, delay onset, reduce severity or incidence, or yield prophylaxis of one or more symptoms or aspects of the viral disease, disorder, or condition. For the purposes of the present invention, treatment can be administered before, during, and/or after the onset of symptoms. In certain embodiments, treatment occurs after the subject has received conventional therapy. In some embodiments, the term “treating” includes abrogating, substantially inhibiting, slowing, or reversing the progression of the viral disease or symptoms thereof, substantially ameliorating clinical or aesthetical symptoms of a condition, or substantially preventing the appearance of clinical or aesthetical symptoms of a condition, or decreasing the severity and/or frequency one or more symptoms resulting from the disease.

As used herein, the term “prevent” refers to the prophylactic treatment of a subject who is at risk of contracting the viral disease resulting in a decrease in the probability that the subject will develop the disease.

The term “therapeutic regimen” as used herein refers to the specific order, timing, duration, routes and intervals between administration of one of more therapeutic agents. In one embodiment a therapeutic regimen is subject-specific. In another embodiment, a therapeutic regimen is virus-specific or symptom-specific. In another embodiment, the therapeutic regimen changes as the subject responds to the therapy. In another embodiment, the therapeutic regimen is fixed until certain therapeutic milestones are met. In one embodiment, the therapeutic regimen involves one or more doses of an inhibitor of one such gene, coupled with administration of one or more doses of an inhibitor of another gene required for viral infection. In one embodiment, the one or more doses of a first inhibitor is administered simultaneously or sequentially with one or more doses of a second or additional inhibitor (the term additional meaning any number of different inhibitors of a different gene. In another embodiment, the therapeutic regimen involving the combination of a first inhibitor and a second or additional inhibitor involves administering the first inhibitor less frequently than that inhibitor would be administered if it was administered as a sole therapeutic agent. In another embodiment, the therapeutic regimen involving the combination of inhibitors of the activity or expression of different host genes required for infection involves administering all combined inhibitors less frequently than the inhibitors would be administered if they were administered as sole therapeutic agents.

By “therapeutic effect” or “treatment benefit” as used herein is meant an improvement or diminution in severity of a symptom of the viral disease, for example, an increase in ease of breathing, a decrease in fever or inflammation, etc, including an improvement or diminution in severity of treatment side effect, or reduction the dosage amount or repetition or duration of administration of a therapeutic composition or agent, e.g., less frequent administration of a particular inhibitor. In one embodiment, the therapeutic benefit of the two or more inhibitors of one or more host genes is enhanced relative to the administration of any inhibitor alone. In another embodiment, the therapeutic benefit is a synergistic therapeutic effect or treatment benefit, e.g., a benefit more than would be expected by the treatment benefits of each inhibitor of Table III or Table IV additively. In another embodiment, the administration of multiple inhibitors produces improved tolerability of one or more inhibitors. In another embodiment, the administration of multiple inhibitors of one or more of the genes or gene products described herein permits use of a dosage amount of at least one inhibitor that is lower than the dose approved for single agent use. In a further embodiment, the administration of multiple inhibitors of multiple of the genes or gene products or activity described herein enhances the duration of the therapeutic effect or treatment benefit achieved with any inhibitor administered alone.

An “effective amount” is meant the amount of the inhibitors, e.g., those specified in Tables III or IV, taken alone, or in combination or with other conventional anti-viral medicaments, sufficient to provide a therapeutic benefit or therapeutic effect after a suitable course of administration.

It should be understood that the “effective amount” for an inhibitor varies depending upon the inhibitor/antagonist selected for use in the method. Regarding doses, it should be understood that “small molecule” drugs are typically dosed in fixed dosages rather than on a mg/kg basis. With an injectable a physician or nurse can inject a calculated amount by filling a syringe from a vial with this amount. In contrast, tablets come in fixed dosage forms. Some dose ranging studies with small molecules use mg/kg, but other dosages can be used by one of skill in the art, based on the teachings of this specification. In one embodiment an effective amount for the selected inhibitor(s) includes without limitation about 0.001 to about 25 mg/kg subject body weight. Such a range includes 0.001, at least 0.01, at least 0.1, at least 1, at least 10, at least 20, or at least 25 mg/kg body weight. In another embodiment, the range of effective amount is 1 to 5 mg/kg body weight. In another embodiment, the range of effective amount is 1 to 10 mg/kg body weight. In another embodiment, the range of effective amount is 1 to 20 mg/kg body weight. Still other doses falling within these ranges are expected to be useful. Where the inhibitor is a protein, e.g., antibody, antibody fragment or recombinant protein or peptide, the effective amount can be about 0.01 to 25 mg antibody/injection. In one embodiment, the effective amount is 0.01 to 10 mg antibody/injection. In another embodiment, the effective amount is 0.01 to 1 mg antibody/injection. In another embodiment, the effective amount is 0.01 to 0.10 mg antibody/injection. In another embodiment, the effective amount is 0.2, 0.5, 0.8, 1.0, 1.2, 1.4, 1.6, 1.8, 2.0, 2.2, 2.4, 2.6, 2.8, 3.0 up to more than mg antibody/injection. Still other doses falling within these ranges are expected to be useful.

In another embodiment of the combination methods of this invention, the effective amount is a “low dose” of inhibitor. Such a low dose is defined as a dose lower than that used if the inhibitor were to be administered alone or for another condition. In another embodiment, the lose dose is less than 1 mg/kg subject body weight. In one embodiment, the low dose is less than 0.1 mg/kg subject body weight. In one embodiment, the low dose is less than 0.01 mg/kg subject body weight. In one embodiment, the low dose is less than 0.001 mg/kg subject body weight. In one embodiment, the low dose is between 0.001 and 0.0001 mg/kg subject body weight. In one embodiment, the low dose is a normal dose administered less frequently than a monotherapy dose, e.g., less than once a day. A low dose for the inhibitor in one embodiment, can be from 0.01 to 25 mg antibody/injection, with the injections administered at extended intervals.

Low doses can also include regimens of administration where the dosages are administered over extended periods of time in comparison to known anti-viral doses and regimens. In one embodiment, a dosage of one or more inhibitors can be administered daily, weekly, bi-weekly, monthly, or for as long as the virus persists in the subject. Further when in a vaccine composition, effective dosages can be administered once every 6 months or once/year, as needed.

In one embodiment, the dose and dosage regimen of the selected inhibitor(s) that is suitable for administration to a particular patient may be determined by a physician considering the patient's age, sex, weight, general medical condition, and whether the inhibitor is administered to prevent viral infection or to treat viral infection and its symptoms. The physician may also consider the route of administration of the agent, the pharmaceutical carrier with which the agents may be combined, and the agents' biological activity.

By “administration” or “routes of administration” include any known route of administration that is suitable to the selected inhibitor or inhibitors, and that can deliver an effective amount to the subject. Routes of administration useful in the methods described herein include one or more of oral, parenteral, intravenous, intra-nasal, sublingual, intraocular injection, intravitreal injection, via a depot formulation or device, by inhalation, or any other route suitable to the selected inhibitor.

### F. Pharmaceutical Compositions

Pharmaceutical compositions may include one or more of the inhibitors identified herein contained in a single composition comprising at least one carrier (e.g., pharmaceutically acceptable carrier). Alternatively, multiple inhibitors may be administered separately (e.g., administered in separate compositions comprising at least one carrier (e.g., pharmaceutically acceptable carrier)). The pharmaceutical preparations containing the inhibitors may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the inhibitors to be administered, its use in the pharmaceutical preparation is contemplated.

In a particular embodiment, multiple inhibitors may be administered sequentially and/or concurrently. For example, an inhibitor of one host gene may be administered before, after, and/or at the same time as an inhibitor of another host gene. When the inhibitor compositions are not administered at the same time, the compositions should be administered close enough in time such that the two compositions are capable of acting synergistically, additively or in a manner to achieve a treatment benefit in the subject.

Selection of a suitable pharmaceutical preparation depends upon the method of administration chosen. For example, inhibitor compositions may be administered parenterally by intravenous injection into the blood stream, or by subcutaneous, intramuscular or intraperitoneal injection. Pharmaceutical preparations for parenteral injection are known in the art. If parenteral injection is selected as a method for administering the antibodies, steps must be taken to ensure that sufficient amounts of the molecules reach their target cells to exert a biological effect. The lipophilicity of the agents, or the pharmaceutical preparation in which they are delivered, may be increased so that the molecules can better arrive at their target locations.

Pharmaceutical compositions containing the inhibitors/antagonists described herein as the active ingredient in intimate admixture with a pharmaceutical carrier can be prepared according to conventional pharmaceutical compounding techniques. The carrier may take a wide variety of forms depending on the form of preparation desired for administration. In preparing the agent in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric coated by standard techniques. For parenteral compositions, the carrier will usually comprise sterile water, though other ingredients, for example, to aid solubility or for preservative purposes, may be included. Injectable suspensions may also be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of the compositions of the invention may be determined by evaluating the toxicity of the active therapeutic inhibitor in animal models. Various concentrations of the above-mentioned inhibitors including those in combination may be administered to a mouse model of the viral disease, and the minimal and maximal dosages may be determined based on the results of significant reduction of fever, shortness of breath, and side effects as a result of the treatment. Appropriate dosage unit may also be determined by assessing the efficacy of the inhibitor compositions in combination with other standard drugs for treatment or prophylaxis of viral diseases. The dosage units of the inhibitors may be determined individually or in combination.

The compositions comprising the inhibitors of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The present invention also includes pharmaceutical kits useful, for example, in the treatment or prevention of viral infection or symptoms referred to herein which include one or more containers containing one or more selected inhibitors in therapeutically effective amounts or for administration according to a desired therapeutic regimen. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit. Components that permit these efficacy studies can also be included in the kits.

In the descriptions of the compositions and methods discussed herein, the various components can be defined by use of technical and scientific terms having the same meaning as commonly understood by one of ordinary skill in the art to which this invention belongs and by reference to published texts. Such texts provide one skilled in the art with a general guide to many of the terms used in the present application. The definitions contained in this specification are provided for clarity in describing the components and compositions herein and are not intended to limit the claimed invention.

Throughout this specification, the words “comprise”, “comprises”, and “comprising” are to be interpreted inclusively rather than exclusively. The words “consist”, “consisting”, and its variants, are to be interpreted exclusively, rather than inclusively. It should be understood that while various embodiments in the specification are presented using “comprising” language, under various circumstances, a related embodiment is also described using “consisting of” or “consisting essentially of” language.

The term “a” or “an”, refers to one or more, for example, “a gene,” is understood to represent one or more genes. As such, the terms “a” (or “an”), “one or more,” and “at least one” are used interchangeably herein.

As used herein, the term “about” means a variability of 10% from the reference given, unless otherwise specified.

### EXAMPLES

The following examples should be construed to encompass any and all variations that become evident as a result of the teaching provided herein.

### Example 1: Materials and Methods

### Human Cell Culture

Human alveolar basal epithelial adenocarcinoma cells (A549, ATCC CCL-185), human hepatocellular carcinoma (Huh7.5, a kind gift from C. Rice), human colorectal carcinoma (Caco-2, ATCC HTB-37), and human embryonic kidney cells HEK293FT (Thermo) were maintained at 37° C. and 5% CO₂ in D10 media, which consists of DMEM (Caisson Labs) with 10% Serum Plus II Medium Supplement (Sigma-Aldrich). Lung adenocarcinoma (Calu-3, ATCC HTB-55) and monkey kidney epithelial cells (Vero E6, ATCC CRL-1586) were maintained in EMEM (ATCC) media with 10% Serum Plus II Medium Supplement (Sigma-Aldrich).

### Viral Strains

SARS-related coronavirus 2 (SARS-CoV-2), isolate USA-WA1/2020 (NR-52281), was produced as previously described (Blanco-Melo et al., 2020; Daniloski et al., 2020). SARS-CoV-2 was grown in Vero E6 cells in DMEM supplemented with 2% FBS, 4.5 g/L D-glucose, 4 mM L-glutamine, 10 mM non-essential amino acids, 1 mM sodium pyruvate and 10 mM HEPES. Plaque assays were used to determine infectious titers of SARS-CoV-2 by infection of Vero E6 cells in Minimum Essential Media supplemented with 2% FBS, 4 mM L-glutamine, 0.2% BSA, 10 mM HEPES and 0.12% NaHCO3 and 0.7% agar.

A549 overexpressing human ACE2 (A549^{ACE2}) cells (a gift from B. Rosenberg, Icahn School of Medicine at Mount Sinai) were previously described (Bouhaddou et al., 2020). To generate ACE2 expressing cells, the human ACE2 coding sequence was amplified and cloned into the BamHI site of the lentiviral vector pHR-PGK (Addgene 79120). We generated lentiviral particles (as described below) and transduced 5×10⁴ A549 cells plated in a 12-well plate in the presence of polybrene (8 μg/ml). We confirmed hACE2 expression by western blot analysis (Thermo MA5-32307).

### Lentiviral Production, Transduction and SARS-CoV-2 Infection

The Human GeCKOv2 A and B libraries (Addgene, 1000000048) were used for genome-scale CRISPR knockout screens (Sanjana et al., 2014). We mixed equal amount of the A and B library plasmids to target each gene with 6 guide RNAs. Briefly, 225 cm² flasks of 80% confluent HEK293FT cells (Thermo) were transfected with 25 μg GeCKOv2 plasmid, 14 μg pMD2.G and 20 μg psPAX2 mixed in 2.5 mL OptiMEM (Thermo) and 175 μL Polyethylenimine (1 mg/ml) (Polysciences). After 6 hours, media was changed to D10 media with 1% bovine serum albumin (Sigma) added to improve virus stability. After 60 hours, viral supernatants were harvested and centrifuged at 3,000 rpm at 4° C. for 10 min to pellet cell debris and filtered using 45 μm PVDF filters (CellTreat). The supernatant was then ultracentrifuged for 2 hours at 100,000 g (Sorvall Lynx 6000) and the pellet resuspended overnight at 4° C. in PBS with 1% BSA.

Following lentiviral titrations, about 330 million A549^{ACE2} expressing cells were transduced at MOI of 0.5. Cells were selected with 2 μg/μL puromycin (Thermo) for 12 days to ensure proper selection. Throughout the experiment the representation was monitored such that each guide RNA is represented by at least 1000 cells (˜125 M cells). Roughly 1000× representation of cells expressing GeCKOv2 library were infected with SARS-CoV-2 isolate USA-WA1/2020 (NR-52281) at either MOI of 0.01 (low MOI) and 0.3 (high MOI). Surviving cells were collected on day 6 post-infection for genomic DNA isolation.

### Genomic DNA Isolation, Guide RNA Amplification and Quantification

We used a two-step PCR protocol (PCR1 and PCR2) to amplify the guide RNA cassette for Illumina sequencing. Briefly, genomic DNA (gDNA) was extracted using a previously described protocol (Chen et al., 2015). For the first PCR reaction, we used all gDNA available for each sample. We performed the PCR1 using Taq-B polymerase (Enzymatics) and used multiple reactions where each reaction contained up to 10 μg gDNA per 100 μL PCR reaction. PCR1 products for each sample were pooled and used for amplification with barcoded PCR2 primers. For each sample, we performed 12 PCR2 reactions (using 5 μL of the pooled PCR1 product per PCR2 reaction) with Q5 polymerase (NEB). PCR2 products were pooled and then normalized within each biological sample before combining uniquely-barcoded separate biological samples. The pooled product was then gel-purified from a 2% E-gel EX (Life Technologies) using the QiaQuick gel extraction kit (Qiagen). The purified, pooled library was then quantified with Tapestation 4200 (Agilent Technologies). PCR products were run on a 2% agarose gel and the correct size band was extracted. Sequencing was performed on the NextSeq 550 instrument using the HighOutput Mode v2 with 75 bp paired-end reads (Illumina). For PCR1 the following primer set was used: 5′ GAGGGCCTATTTCCCATGATTC 3′ (SEQ ID NO: 1) and 5′ GTTGCGAAAAAGAACGTTCACGG 3′ (SEQ ID NO: 2). For PCR2 the following primers were used: 5′ AATGATACGGCGACCACCGAGATCTACACTCTTTCCCTACACGACGCTCTTCC GATCT (N₁₋₉) (BC₈) TCTTGTGGAAAGGACGAAACACCG 3′ (SEQ ID NO: 3) and 5′ CAAGCAGAAGACGGCATACGAGAT (BC₈) GTGACTGGAGTTCAGACGTGTGCTCTTCCGATCT (N₁₋₉) TCTACTATTCTTTCCCCTGCACTGT 3′, (SEQ ID NO: 4) where N is a stagger of 1 to 9 nucleotides and BC is a barcode of 6 nucleotides. All PCR1 and PCR2 primer sequences, including full barcodes, are listed on the GeCKO website (http://genome-engineering.org/gecko/).

Sequencing reads were demultiplexed upon sequencing based on Illumina i7 barcodes present in PCR2 reverse primers using Illumina BaseSpace. We performed adaptor trimming by treating the hU6 promoter sequence as a 5′ adapter, using cutadapt v1.13 [-e 0.2-O 5-m 20-g TCTTGTGGAAAGGACGAAACACCG] (SEQ ID NO: 5). Processed guide RNA sequences were aligned to the GeCKOv2 reference allowing for up to 1 mismatch using bowtie v1.1.2 [-a-best-strata-v 1-norc] with alignment rates of 81% to 86%.

### Computational Analyses of Genome-Scale CRISPR Screens

Guide RNA counts were processed using the MaGeCK pipeline with an output of RRA p-values and gene ranks (Chen et al., 2018). We separately ranked the gene using the RIGER (weighted-sum) and second-best rank methods, as previously described (Chen et al., 2015). Gene Set Enrichment Analyses were performed using the fgsea package with Gene Ontology for biological processes (c5.bp.v7.1.symbols) (Korotkevich et al., 2019). GTEx v8 tissue specific enrichment was performed using the Multi Gene Query function available on the GTEx website: www.gtexportal.org/home/multiGeneQueryPage (accessed Aug. 1, 2020) (Aguet et al., 2019). GO enrichments for SARS-CoV-2, ZIKA, and IAV CRISPR screens were performed using GOrilla to find all significant enrichments (FDR p-value <10−3) (Eden et al., 2009). FDR p-values were log 10-transformed and normalized to create the heatmap shown in FIG. 3E.

### Generation of Gene-Perturbed A549^{ACE2} Lines and SARS-CoV-2 Infection

For each gene of interest, 3 guides were designed using GUIDES software and were subsequently cloned into an all-in-one vector with an optimized Cas9 scaffold (pCC_01, Addgene 139086) containing Cas9, a guide RNA cassette and a puro resistant cassette (Legut et al., 2020; Meier et al., 2017). Following sequence confirmation by Sanger sequencing, lentivirus was produced individually for each plasmid and the target cells were transduced in presence of polybrene (10 μg/mL, Santa Cruz). Gene-perturbed A549^{ACE2} cells were selected for at least 10 days with 2 μg/mL (A549ACE2, Calu-3), 3 μg/mL (Caco-2) or 5 μg/mL (Huh7.5ACE2) puromycin (Thermo). To determine the SARS-2 infection susceptibility of the A549^{ACE2} gene-perturbed lines, 10,000 cells were plated per well of 96-well plates. After 24 hours, the cells were infected with SARS-CoV-2 at MOI of 0.1. At 36 hours post-infection, the cells were either fixed and processed for immunofluorescence or cellular RNA was harvested for qRT-PCR analyses. All infections with SARS-CoV-2 were performed with 3 biological replicates.

### Immunofluorescence of Nucleocapsid (N) Protein

Cells were fixed with 5% formaldehyde and immunostained for nucleocapsid (N) protein and visualized with a secondary antibody labeled with AlexaFlour-568 (Thermo). SARS-CoV-2 nucleocapsid (N) antibody (clone 1C7C7) was obtained from the Center for Therapeutic Antibody Discovery at the Icahn School of Medicine at Mount Sinai. Nuclei were stained with DAPI. Full wells were imaged and quantified for SARS-CoV-2 infected cells using a Celigo imaging cytometer (Nexcelom Biosciences). All infections with SARS-CoV-2 were performed with 3 biological replicates. Representative images from the top gene knockout hits were acquired using the EVOS M5000 Imaging System (Thermo).

### Identification of Druggable Genes and Drug Treatments

To identify druggable genes among the top CRISPR screen hits, we cross-referenced the top 500 genes from the RRA analysis with the data about drugs and their gene targets obtained from Drug Gene Interaction database (DGIdb, retrieved on Jun. 3, 2020) as well as manual literature search (Cotto et al., 2018). Inhibitors that target the genes of interest (and remdesivir) was obtained from 2 companies: SelleckChem and MedChemExpress. The catalog number and vendor information is available in the Key Resource section.

To test drug efficacy in reducing SARS-CoV-2 infection, 10,000 A549^{ACE2} cells were seeded per well of a 96-well plate. Cells were treated with inhibitors at 10 μM for two hours before infection and inhibitors were maintained throughout the course of infection. Cells were infected with SARS-CoV-2 at MOI of 0.1 for 36 hours and the cells were collected for analysis via qRT-PCR or processed for immunofluorescence (N protein and quantified by Celigo). For FIG. 4D, the remdesivir data was collected in an independent experiment.

### Cell Viability Assays

Cell viability following drug treatments was performed the same way as described above. Thirty-six hours post drug treatment the cells were collected and stained with LIVE-DEAD Violet (Thermo). Cell acquisition was performed using a Sony SH800S cell sorter with a 100 μm sorting chip. We used the following gating strategy: 1) We excluded the cell debris based on the forward and reverse scatter; 2) Doublets were excluded by plotting FSC height vs FCS area, 3) Dead cells were quantified using live-dead violet stain. For all samples, we recorded at least 5,000 cells that pass the gating criteria described above. Flow cytometry analyses were performed using FlowJo v10. Cell viability for A549-ACE2 treated with DMSO or 10 μM amlodipine was performed 24 hours post treatment using Trypan Blue (Thermo Fisher) and automated cell counting (Nexcelom AutoT4).

Quantitative Reverse-Transcription PCR (qRT-PCR) of Viral RNA

RNA was extracted from cells grown in 96-well plates by using the RNeasy 96 Kit (Qiagen) per the manufacturer's instructions. RNA was reverse-transcribed and PCR amplified using KAPA SYBR FAST One-step Universal qRT-PCR Kit (Roche). SARS-CoV-2 replication was assessed by using primers specific to the N mRNA (Forward 5′-CTCTTGTAGATCTGTTCTCTAAACGAAC-3′ (SEQ ID NO: 6); Reverse 5′-GGTCCACCAAACGTAATGCG-3′ (SEQ ID NO: 7)). SARS-CoV-2 N mRNA levels were normalized to beta tubulin (Forward 5′-GCCTGGACCACAAGTTTGAC-3 (SEQ ID NO: 8); Reverse 5′-TGAAATTCTGGGAGCATGAC-3′ (SEQ ID NO: 9)). Reactions were ran and analyzed on a Lightcycler 480 II Instrument (Roche). Relative quantification was calculated by comparing the cycle threshold (Cₜ) values using ΔΔCₜ.

siRNA Transfections and SARS-CoV-2 Infection

All of the siRNAs were ordered from Dharmacon and their catalog number can be found in the Key Resource (data not shown). To knockdown individual genes, 10,000 A549^{ACE2} cells were seeded in 96-well plates and transfected with siRNAs using Lipofectamine RNAiMAX (Thermo) following the manufacturer's protocol. Forty-eight hours later, the cells were infected with SARS-CoV-2 at an MOI of 0.1 for 36 hours. Cells were fixed with 5% formaldehyde, stained with nucleocapsid protein (clone 1C7C7, ISMMS), and visualized with AlexaFluor-568 conjugated secondary antibody (Thermo). Nuclei were stained with DAPI, and full wells were imaged with a Celigo imaging cytometer (Nexcelom Biosciences).

### Minipool CRISPR Library and Lentiviral Production

To generate the minipool, we combined equimolar amounts the same guide RNA vectors (cloned in pCC1) that we used for arrayed validation (see above Generation of gene-perturbed A549^{ACE2} lines and SARS-CoV-2 infection). In total, we combined 3 guide RNAs x 30 genes plus 9 non-targeting guide RNAs. The non-targeting guide RNA plasmids were added at half the molar ratio of the other plasmids. Lentivirus was produced via transfection of the minipool with appropriate packaging plasmids (psPAX2: Addgene 12260; pMD2.G: Addgene 12259) using polyethylenimine (PEI) reagent in HEK293FT. The target A549^{ACE2} cells were transduced in presence of polybrene (10 μg/mL, Santa Cruz) at very low MOI to ensure entry of a single virus per cell. The survival was 2.4% after 2 days of selection with 2 μg/mL puromycin (Thermo). On day 10 post-transduction, the cells were collected and processed for ECCITE-seq.

### Expanded CRISPR-Compatible Cellular Indexing of Transcriptomes and Epitopes (ECCITE-Seq)

We divided the ECCITE-seq pool into several sub-pools and performed cell-hashing. Cell hashing was performed as described in a previously published protocol (Mimitou et al., 2019), Hashed cells were pooled. For each pool, we resuspended 1 million cells in 100 μl staining buffer (2% BSA, 0.01% Tween in PBS). We then added 10 μl Human TruStain FcX Fc Receptor Blocking Solution (BioLegend) and incubated on ice for 10 minutes. We then added hashing antibodies (BioLegend), incubated on ice for 30 minutes, and washed cells 3 times with staining buffer. In total, we ran four lanes of a Chromium Single Cell Immune Profiling Solution v1.0 5′ kit (10× Genomics) targeting recovery of 12,000 cells per lane (superloading) in experiment 1 and 20,000 cells in experiment 2. Gene expression (mRNA), hashtags (Hashtag-derived oligos, HTOs) and guide RNA (Guide-derived oligos, GDOs) libraries were constructed (Smibert et al., 2019). Each replicate was sequenced on two NextSeq550 75-cycle high-output runs (Illumina). Sequencing reads from the mRNA library were mapped to the hg38 reference genome (Ensembl v97) using Cellranger (v3.0.1, 10× Genomics). To generate count matrices for HTO and GDO libraries, the CITE-seq-count package was used (https://github.com/Hoohm/CITE-seq-Count v1.4.2). Count matrices were then used as input into the Seurat R package (v3.2) for downstream analyses (Stuart et al., 2019). Cell viability was determined to be ˜95%, and then cells were profiled using Chromium Single Cell 5′ Solution (10× Genomics).

### ECCITE-Seq Library Construction and Sequencing

For the ECCITE-seq experiment, we ran two lanes of a 10× Genomics 5′ kit (Chromium Single Cell Immune Profiling Solution v1.0, #1000014, #1000020, #1000151) aiming for recovery of 12,000 cells per lane (superloading). Gene expression (mRNA), hashtags (Hashtag-derived oligos, HTOs), protein (Antibody-derived oligos, ADTs) and guide RNA (Guide-derived oligos, GDOs) libraries were constructed by following 10× Genomics and ECCITE-seq protocols. All libraries were sequenced together on two NextSeq550 75-cycle high-output runs (Illumina). Sequencing reads from the mRNA library were mapped to the hg38 reference genome (Ensembl v97) using Cellranger (v3.0.1, 10× Genomics). To generate count matrices for HTO and GDO libraries, the CITE-seq-count package was used (https://github.com/Hoohm/CITE-seq-Count v1.4.2). Count matrices were then used as input into the Seurat R package (v3.2) for downstream analyses (Stuart et al., 2019).

### Data Pre-Processing in Seurat

Cells with low quality metrics, high mitochondrial gene content (>17.5%) and low number of genes detected (≤1800) were removed. The median number of detected genes was 3309. RNA counts were log-normalized using the standard Seurat workflow. HTO counts were normalized using the centered log-ratio transformation approach, with margin-2 (normalizing across cells). To identify cell doublets and assign experimental conditions to cells, we used the HTOseqDemux function (Stoeckius et al., 2018). HTOseqDemux-defined cell doublets and negatives were removed from any downstream analyses.

Cellular guide RNA identity for cells in the ECCITE-seq pool was assigned based on GDO unique molecular identifier (UMI) counts. We considered a guide RNA detected with ≥16 μMI counts (the median UMI counts per guide RNA was 503 counts, 93% of cells with ≥1 sgRNA). We observed that 64% of the cells had >1 detected guide RNA (29% 1 guide RNA, 27% 2 guide RNAs, 17% 3 guide RNAs), which is surprising given a MOI <0.05 and an experimentally determined 2.4% survival rate upon selection (single integration probability of ˜98% assuming Poisson statistics). To maximize cell recovery, we retained cells with 1-3 guide RNAs per cell (n=10,265) for downstream analysis but with several stringent filtering steps that we describe in detail.

First, we collapsed the guide RNA information down to target gene level (3 guide RNA per target gene) in the following way: 1) For all cells (with 1-3 guide RNA), we assigned a unique target gene if all detected guide RNAs targeted the same gene, or if the additional guide RNA detected was a non-targeting (NT) guide RNA. 2) For remaining cells, we assigned 2 or 3 target genes. Next, we removed all cells with 3 guide RNAs targeting 3 separate genes. We also removed cells if the combination of 2 unique target genes contained guide RNAs for 2 genes that were classified as ‘perturbed’ (see below) to avoid mixing of transcriptomic perturbation signatures. Similarly, we removed cells with a combination of guide RNAs for 2 unique target genes, if cells with guide RNAs for both genes were classified as ‘non-perturbed’ to avoid introduction of genetic interaction effects for gene KOs that do not lead to an RNA perturbation signature on their own. Lastly, we only retained cells with 2 unique target genes, if cells with guide RNAs for one of the 2 target genes could be classified as ‘perturbed’, while the other retained a non-perturbed (‘escaping’) classification. For this last case, we tested and verified that the second target gene does not contribute to the observed RNA perturbation signature. Based on this selection process, we assigned the target gene label based on 1) the uniqueness of the target gene by guide RNA labels, or 2) based on the driving target gene (1 target gene classified as ‘perturbed’, while the other was classified ‘non-perturbed’, see below). This filtering process yielded 6,522 cells total with the majority (4,715 cells) containing a single best guide RNA or unique target gene.

### RNA-Based Clustering of Single Cells and ECCITE-Seq Data

ECCITE-seq single-cell clustering was done in a multistep process. First, we classified cells based on their perturbation status to discriminate between ‘perturbed’ cells (cells detectable with guide RNA-dependent perturbation signature/gene expression changes) or ‘escaping’ (non-perturbed) cells that can occur if the Cas9 introduced mutation was inconsequential or if loss of the target gene did not lead to transcriptomic changes. We classified cells to be perturbed or non-perturbed relative to cells with non-targeting (NT) guide RNAs using mixscape with default parameters (Papalexi et al., 2020).

In brief, we calculated the local perturbation signature to remove confounding sources of variation (e.g. cell cycle) by subtracting the average expression of the k=20 nearest NT neighbors from each cell's original RNA profile using the first 40 principal components. The perturbation signatures were centered but not scaled using the ScaleData function. Next, we used RunMixscape, which determines for each target gene class, all of the differentially expressed genes (DEGs) relative to NT cells on the original RNA counts. Then RunMixscape calculates the perturbation score of every cell by using the DEG perturbation signatures and projected cells into a single dimension. Lastly, a Gaussian mixture model is fitted to the distribution of perturbation scores of NT and each target gene class separately, with the assumption that non-perturbed (‘escaping’) cells cannot be discriminated from NT cells, while ‘perturbed’ cells group separately. In this way, all cells targeting a gene for which no DEGs could be identified (min.de.genes=5 with log fc.threshold=0.25) were classified as non-perturbed. For all other target genes with DEGs, all cells that had a similar perturbation score with NT cells were classified as non-perturbed.

We first applied mixscape to all cells with a sgRNAs targeting a unique target gene (n=4,715 cells). We identified perturbed cells for 11 out of 30 target genes. We then added cells with sgRNA combination of a perturbed target gene and a non-perturbed target gene as described above (n=6,522 cells) and repeated the classification. Again, we identified perturbed cells for 11 out of 30 target genes. We removed all non-perturbed (‘escaping’) cells. Downstream analyses were conducted using this set of n=2,023 perturbed cells and n=352 NT cells. For visualization, we performed linear discriminate analysis (LDA) using the mixscape-derived class labels and performed UMAP dimensionality reduction using all (n=11) component shown in FIG. 5B.

### Differential Expression and Gene Set Enrichment Analyses

We used FindMarkers in Seurat to find differentially expressed genes between non-targeting cells and cells that belonged to a targeted gene class. We used all genes with adjusted p-value <0.01 and minimal logₑFC threshold >0.1 filter as input into the heatmap in FIG. 5C. The same FindMarkers analysis (but using the non-adjusted p-value <0.01 with a maximum of the 300 most significant DEGs) was used as input into the EnrichR package to run pathway analysis using the human WikiPathways database (v. 2019) (Kuleshov et al., 2016; Slenter et al., 2018). FIG. 5D shows-log₁₀ transformed p-values for the union of all enriched pathways with an adjusted p-value <0.01 across the 11 target genes with perturbation signatures.

### Flow Cytometry of ACE2 Cell Surface Expression

Cells were harvested, counted and about 100,000 cells were washed with Dulbecco's phosphate-buffered saline (PBS, Caisson Labs) and then stained with LIVE/DEAD Violet stain (Thermo, 34864). Following a wash with 1×PBS, all subsequent washes and antibody dilutions were performed using 1× PBS supplemented with 2% FBS. Wild-type or Cas9-perturbed A549^{ACE2}, Caco-2 and Calu-3 cells were stained for 30 min on ice with 0.25 μg of anti-ACE2 antibody (R&D Systems, AF933) in ˜50 μL residual volume. Following two washes with 1×PBS with 2% FBS, samples were stained on ice for 20 min with 7.5 μL of anti-goat-APC secondary antibody (R&D Systems, F0108). Cell acquisition and sorting was performed using a Sony SH800S cell sorter with a 100 μm sorting chip. We used the following gating strategy: 1) We excluded the cell debris based on the forward and reverse scatter; 2) Doublets were excluded by plotting FSC height vs FCS area, 3) Dead cells were excluded by live-dead stain. We recorded at least 5,000 cells for A549ACE2 and at least 3,000 cells for Calu-3 and Caco-2 that pass the gating criteria described above. Gates to determine ACE2-APC+ cells were set based on control A549 wild type cells or only secondary antibody stained Calu-3 and Caco-2, where the percent of ACE2 positive cells was set as <5% (background level). Flow cytometry analyses were performed using FlowJo v10.

### Immunofluorescence of ACE2 and Endo/Lysosomal Markers

A549^{ACE2} Cas9-transduced (specific gene or non-targeting) cell lines were seeded on poly-D-lysine-coated coverslips (Electron Microscopy Sciences) 24 hours before they were fixed with 4% formaldehyde (Sigma) diluted in 1× PBS (Caisson Labs) (Daniloski et al., 2020). Following 3 washes with 1×PBS, cells were blocked with 1×PBS with 2% BSA for 30 min at room temperature. Cells were stained with mouse anti-2A antibody diluted at 1:250 (clone 3H4, Millipore Sigma, MABS2005) to recognize ACE2-2A, and rabbit anti-EEA1 diluted at 1:100 (Thermo, MA5-14794). Cell were incubated with primary antibodies for 3 hours at room temperature in a moisturized chamber. Coverslips were then washed 5 times with 1×PBS with 2% BSA and then incubated with Alexa-conjugated secondary antibodies (Jackson ImmunoResearch) diluted at 1:1000 for 45 min at room temperature. Coverslips were then washed 5 times with 1×PBS with 2% BSA, and DAPI (Sigma) was added in the fourth wash at a concentration of 0.1 μg/mL. To stain the lysosomes, LysoTracker DeepRed (Thermo) was diluted to 70 nM in culture media and was added to actively growing cells on coverslips and were incubated for 1 hour at 37° C.

Following incubation with LysoTracker, cells were processed for immunofluorescence as described above. Confocal images were acquired on a Zeiss LSM 780 with a 20×/0.8 Plan-APOCHROMAT objective or a 63×/1.4 Plan-APOCHROMAT objective (Zeiss). Epifluorescence images were acquired using the same system; both fitted with a Zeiss Axiocam 506 mono (Zeiss). Images were processed using Zen Black 2012 (Zeiss) and FIJI 2.1.0; Java 1.8.0-202. Linear histogram adjustments were applied uniformly within experiments for clarity of presentation.

ACE2 localization was determined on images taken on a Zeiss LSM 780 with a 20×/0.8 Plan-APOCHROMAT objective using Zen Black (Zeiss). The A549-ACE2 polyclonal cell line had both ACE2 positive and negative cells. Only ACE2 positive cells were then manually scored if they had a distinct accumulation of cytoplasmic vesicles-like hollow structures, compared to the non-targeting (NT) control. In the NT control, the ACE2 staining was seen on the plasma membrane and diffuse in the cytoplasm. The scoring was performed on 2 biological replicates (that is, two separate non-targeting controls and two separate Rab7A knockout lines engineered using 4 separate Cas9 guide RNAs). We scored ˜430 cells per replicate.

### Western Blots

A549^{ACE2} or Huh7.5^{ACE2} cells were collected, washed with 1×PBS and lysed with TNE buffer (10 mM Tris-HCl, pH 7.4, 150 mM NaCl, 1 mM EDTA, 1% Nonidet P-40) in presence of protease inhibitor cocktail (Bimake B14001) for 1 hour on ice. Cells lysates were spun for 10 min at 10,000 g, and protein concentration was determined with the BCA assay (Thermo). Equal amounts of cell lysates (20 μg) were denatured in Tris-Glycine SDS Sample buffer (Thermo LC2676) and loaded on a Novex 4-20% Tris-Glycine gel (Thermo XP04205BOX). PageRuler pre-stained protein ladder (Thermo 26616) was used to determine the protein size. The gel was run in 1× Tris-Glycine-SDS buffer (IBI Scientific IBI01160) for about 120 min at 120V. Proteins were transferred on a nitrocellulose membrane (BioRad 1620112) in presence of prechilled 1× Tris-Glycine transfer buffer (FisherSci LC3675) supplemented with 20% methanol for 100 min at 100V. Immunoblots were blocked with 5% skim milk dissolved in 1×PBS with 1% Tween-20 (PBST) and incubated overnight at 4° C. separately with the following primary antibodies: rabbit anti-RAB7A (0.1 μg/mL, NovusBio, NBP1-87174), rabbit anti-GAPDH (0.1 μg/mL, Cell Signaling, 2118S), Following the primary antibody, the blots were incubated with IRDye 680RD donkey anti-rabbit (0.2 μg/mL, LI-COR 926-68073) or with IRDye 800CW donkey anti-mouse (0.2 μg/mL, LI-COR 926-32212). The blots were imaged using Odyssey CLx (LI-COR).

### T Cell Isolation and Activation

Human primary T cells were sourced from a de-identified healthy donor's LeukoPaks (New York Blood Center). First, peripheral blood mononuclear cells (PBMCs) were isolated via gradient centrifugation using Lymphoprep (StemCell Technologies 07811). CD8+ T cells were isolated from PBMCs by positive magnetic selection with the EasySep Human CD8 Positive Selection Kit (StemCell Technologies 17853). CD4+ T cells were isolated from PBMCs by negative magnetic selection with the EasySep Human CD4 T cell Isolation Kit (StemCell Technologies 17952). Isolated T cells were immediately plated in ImmunoCult-XF T Cell Expansion Medium (StemCell Technologies 10981) supplemented with 10 ng μL⁻¹ recombinant human IL-2 (StemCell Technologies 78036) at 1×10⁶ cells mL⁻¹. T cells were then activated with the ImmunoCult Human CD3/CD28 T Cell Activator (StemCell Technologies 10971) and expanded for 14 days. At days 3, 5 and 7 post-activation, T cells were diluted to 2.5×10⁵ cells mL⁻¹ with fresh ImmunoCult-XF T Cell Expansion Medium supplemented with IL2. At days 9, 11 and 13 post-activation, T cells were maintained at a density of 2×10⁶ cells mL⁻¹, and half of the culture media was replaced with fresh media supplemented with IL2. At day 14 post activation, T cells were washed off to remove IL2 and rested overnight in ImmunoCult-XF T Cell Expansion Medium prior to the activation assay.

T cell activation was measured by intracellular cytokine staining following anti-CD3/CD28 antibody stimulation. First, CD4+ and CD8+ T cells were pre-incubated for 1 hour at 37° C. with selected inhibitors at 10 μM concentration, or with vehicle control (DMSO). Each inhibitor was tested in triplicate. Following pre-incubation, T cells were activated with the ImmunoCult Human CD3/CD28 T Cell Activator, according to manufacturer's recommendations. 5 μg mL⁻¹ brefeldin A (BioLegend 420601) and 2 μM monensin (BioLegend 420701) were added alongside the T Cell Activator to accumulate the cytokines inside the cell. After 4 h of activation at 37° C., CD4+ and CD8+ T cells were combined for staining, washed with PBS and stained with LIVE/DEAD Fixable Violet Dead Cell Stain Kit (Thermo Fisher Scientific L34955) for 5 minutes at room temperature in the dark. Then, FITC anti-human CD8a (clone RPA-T8, BioLegend 301014) was added followed by 20 min incubation on ice. The cells were then washed with PBS, fixed and permeabilized using Fixation Buffer (BioLegend 420801). Fixation was performed for 20 min on ice. Then, the cells were washed with Intracellular Staining Permeabilization Wash Buffer (BioLegend 421002) and stained with PE/Cy7 anti-human TNF-α (clone MAb11, BioLegend 502930) and PE anti-human IFN-γ (clone 4S.B3, BioLegend 502508) antibodies. The staining was performed for 20 min on ice. Afterwards, the cells were washed and resuspended in the Intracellular Staining Permeabilization Wash Buffer.

Acquisition was performed using Sony SH800S Cell Sorter with a 100 μm sorting chip. Cells were sequentially gated to 1) include leukocytes based on forward and side scatters, 2) exclude doublets, 3) exclude dead cells and 4) include CD8+ cells. At least 10,000 viable CD8+ events were collected per sample.

Inflation of COVID-19 GWAS Signal for Variants that are Cis-eQTLs

To estimate the importance of CRISPR screen hit genes in susceptibility to COVID-19 in humans, we tested for inflation of COVID-19 GWAS signal for variants that are eQTLs in GTEx v8 lung for highly-ranked genes enriched in the CRISPR screens (n=588 genes found in the top 500 genes by at least 2 analysis methods of RRA, RIGER, and second-best ranks for at least one screen MOI) (Aguet et al., 2019). We used summary statistics of the six COVID-19 GWAS generated by the COVID-19 Human Genetics Initiative (round 3 meta-analyses, released Jul. 2, 2020): 1) ANA_A2_V2-very severe respiratory confirmed COVID vs. population, 2) ANA_B1_V2-hospitalized COVID vs. not hospitalized COVID, 3) ANA_B2_V2-hospitalized COVID vs. population, 4) ANA_C1_V2-COVID vs. lab/self-reported negative, 5) ANA_C2_V2-COVID vs. population, 6) ANA_D1_V2-predicted COVID from self-reported symptoms vs. predicted or self-reported non-COVID (COVID-19 Host Genetics Initiative, 2020). We measured inflation using the lambda inflation statistic relative to the chi-square quantile function of 0.1, λ_{0.1}, i.e. estimating the inflation among 10% of the most significant tests. We calculated λ_{0.1} for two sets of eQTLs: 1) all lead eQTLs in lung (14,113 genes with significant eQTLs in lung at FDR 5%, serving as a background set), 2) all lead eQTLs in lung for CRISPR hit genes (274 out 588 top-ranked genes have eQTLs in lung at FDR <0.05).

To test the significance of λ_{0.1}, we used a permutation-based test. We selected n lead eQTLs from the background set k=10,000 times, where n is the number of lead eQTLs for CRISPR hit genes tested in a given COVID-19 GWAS and calculated λ_{0.1} on the permuted data. We calculated permutation p-values as the proportion of permuted λ_{0.1} as extreme as or more extreme than the observed λ_{0.1.}

Prioritization of Genes in the 3p21.31 Locus Based on eQTL Data

We focused on eight genes, LIMD1, SACM1L, SLC6A20, LZTFL1, CCR9, FYCO1, CXCR6, and XCR1, in the 3p21.31 locus that has been shown to robustly associate with COVID-19 susceptibility (COVID-19 Host Genetics Initiative, 2020; Ellinghaus et al., 2020). We gathered summary statistics for 110 eQTL data sets from the eQTL Catalogue (61 data sets, mostly in immune cell types with and without treatment) (Kerimov et al., 2020) and GTEx v8 (49 tissues) (Aguet et al., 2019). We first prioritized the genes based on the p-value in eQTL studies for the lead GWAS variant 3:45867022:C:G from the COVID vs population GWAS (ANA_C2_V2, largest COVID-19 GWAS to date) generated by the COVID-19 HGI. Since observing low eQTL p-value for the lead GWAS variant does not necessarily translate into shared genetic causality, we further performed colocalization analysis.

### Colocalization Analysis of COVID-19 GWAS and Cis-eQTLs in the 3p21.31 Locus

To assess evidence for shared causal variant of a cis-eQTL and a COVID-19 GWAS, we used the Bayesian statistical test for colocalization, coloc (Giambartolomei et al., 2014), assuming one causal variant per trait. We only included cis-eQTLs for genes for colocalization test, if there was a cis-eQTL with a nominal p-value <10⁻⁴ within 100 kb of the lead ANA_C2_V2 COVID-19 GWAS variant. Coloc was run on a 1 Mb region centered on the lead GWAS variant (+/−500 kb from the variant) with prior probabilities set to p₁=10⁻⁴, p₂=10⁻⁴, p3=5×10⁻⁶. We defined suggestive support for colocalization between the ANA_C2_V2 COVID-19 GWAS and cis-eQTL signal if posterior probability for one shared causal variant (PP4)>0.25.

Allelic heterogeneity of gene expression in cis is widespread (Aguet et al., 2019), and it violates the assumption of one causal variant per trait. Thus, we also used the development version of coloc (github.com/chr1swallace/coloc/tree/condmask) (Wallace, 2020) in a wider 2 Mb region centered at the lead ANA_C2_V2 COVID-19 GWAS variant. The enhanced version of coloc allows conditioning and masking to overcome the single causal variant assumption. For eQTL datasets from the eQTL Catalogue, we used method=mask with LD data from the 1000 Genomes Project (1000 Genomes Project Consortium et al., 2015) CEU population that matches the genetic ancestry of the population in majority of the studies in the eQTL Catalogue. For eQTL data sets from the GTEx Project, we used method=cond with LD calculated from the individuals that had gene expression data in the given tissue in GTEx. We used the mode=iterative and allowed for a maximum of three variants to condition/mask. We set the r² threshold to 0.01 to call two signals independent when masking, and we set the p-value threshold to 10⁻⁴ to call the secondary signal significant. We used method=single for the ANA_C2_V2 COVID-19 GWAS.

As a result, the maximum PP4 (posterior probability for shared causal variants) estimates with conditioning/masking were similar to PP4 estimates from the standard coloc run, suggesting no additional colocalization events with secondary independent cis-eQTLs.

### Regional Association Plots for the 3p21.31 Locus

Immunofluorescence co-stained for ACE2, EEA1 and Lysotracker in A549ACE2 cells with a CRISPR guide RNA targeting RAB7A revealed that ACE2 shows a distinct colocalization with EEA1 (an early endosome marker) and a less frequent colocalization with Lysotracker (a lysosomal maker) (data not shown). Data points in the locuszoom-like regional association plots as shown in FIG. 6C are colored by the LD between the lead ANA_C2_V2 COVID-19 GWAS variant 3:45867022:C:G and other variants in the region. For plotting data for COVID-19 GWAS and eQTLs from the eQTL Catalogue, we used genotype data from the 1000 Genomes Project, and calculated a weighted average r² value based on the counts of global ancestral populations in the analysis (if multiple global ancestry populations were studied). Note that for European populations, we only included non-Finnish populations in the 1000 Genomes Project. For plotting data for eQTLs from the GTEx project, we used genotype data available via dbGaP, accession phs000424.v8, and calculated r² using the specific individuals that had expression data from the given tissue.

### Quantification and Statistical Analysis

General data analysis—In all cases, we have mentioned specific software tools used in the respective section of the Methods. All other data analysis was performed in GraphPad Prism 8 and RStudio.

Data representation—In all boxplots, boxes indicate the median and interquartile ranges, with whiskers indicating either 1.5 times the interquartile range, or the most extreme data point outside the 1.5-fold interquartile. All transfection experiments show the mean of three replicate experiments, with error bars representing the standard error of mean.

### Results—a High-Throughput Screen to Identify Genes Required for SARS-CoV-2 Infection

To identify key genes required for SARS-CoV-2 infection, we performed a genome-scale loss-of-function targeting 19,050 genes in the human genome using the GeCKOv2 CRISPR-Cas9 library (Sanjana et al., 2014). The GeCKOv2 library contains 122,411 CRISPR single-guide RNAs (6 guide RNAs per gene) and has previously been used in several previous CRISPR screens, including for drug resistance, immunotherapy, synthetic lethality, mitochondrial disease, and therapeutic discovery for muscular dystrophy (Erb et al., 2017; Jain et al., 2016; Lek et al., 2020; Patel et al., 2017; Shalem et al., 2014). First, we transduced a human alveolar basal epithelial cell line (A549) that constitutively expresses ACE2 (referred to as A549ACE2) with an all-in-one lentiviral vector containing Cas9, guide RNAs from the GeCKOv2 human library, and a puromycin resistance gene. The transduction was performed at a low multiplicity of infection (MOI ˜0.2) to ensure that most cells would receive only one guide RNA construct (FIG. 1A). We then selected with puromycin so that only library-transduced cells remained. We also measured the survival rate after puromycin selection was complete (3 days) to ensure high coverage of the 122,411 guide RNAs (˜1,000 cells per guide RNA). After puromycin selection was complete, we cultured the cells for 9 days to ensure protein depletion after CRISPR gene targeting.

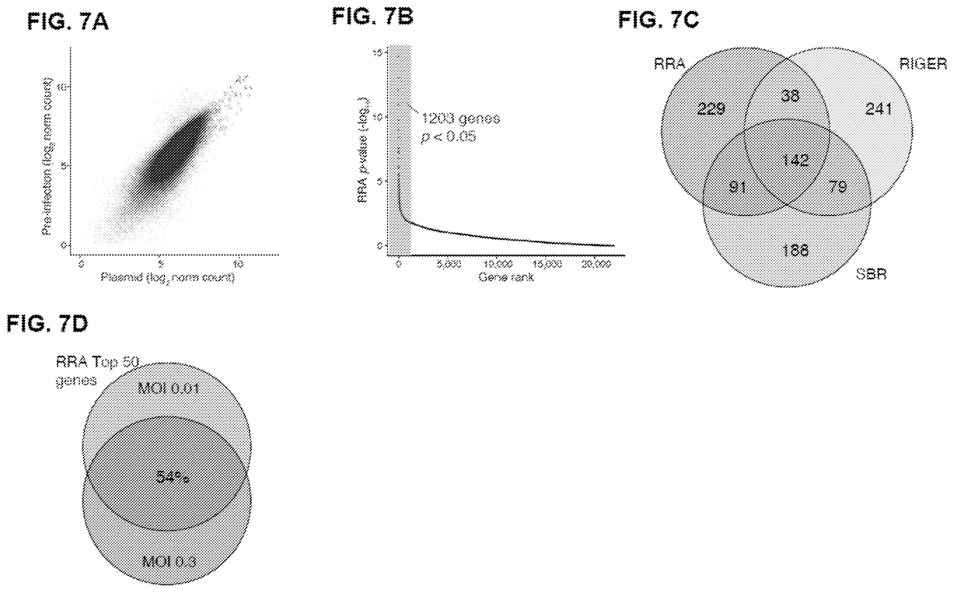

Next, we infected the GeCKOv2 pool of A549ACE2 cells with SARS-CoV-2 virus (Isolate USA-WA1/2020 NR-52281) at either a high (0.3) or a low (0.01) MOI. We verified that SARS-CoV-2 infects A549ACE2 cells by staining for the nucleocapsid (N) protein at 24 hours post-infection (data not shown) and, at day 6 post-infection, we measured cell survival for both the high and low MOI conditions (FIG. 1B). As expected, the higher MOI infection resulted in fewer surviving cells at day 6 post-infection. Next, we extracted genomic DNA and, via amplicon sequencing, we quantified guide RNA abundance in each biological condition (FIG. 1A). To confirm that library representation was properly maintained, we computed the correlation between the guide RNA representation in the plasmid library and after puromycin selection (r=0.84) (FIG. 7A). In contrast, after SARS-CoV-2 infection, there was a much greater degree of guide RNA dropout, as expected given that SARS-CoV-2 rapidly kills A549ACE2 cells without CRISPR perturbations (FIGS. 1C, D).

Using robust-rank aggregation (RRA) on the guide relative enrichments, we computed gene-level scores to identify genes where loss-of-function mutations led to enrichment within the pool (FIG. 1D) (Kolde et al., 2012). We identified approximately 1,000 genes with significant RRA enrichment (p<0.05) (FIG. 7B). We also used two other previously published methods to compute gene enrichments (RIGER weighted-sum and second-best rank) and found a high degree of overlap between enriched genes identified by all three methods (FIG. 7C) (Chen et al., 2015; Luo et al., 2008). We also found a high degree of shared genes across both the low and high SARS-CoV-2 MOI conditions: When examining the top 50 most enriched genes, we found that 27 of them were shared between the low and high MOI conditions (FIG. 7D), suggesting that several host genes involved in viral pathogenesis function independently of viral dose.

### Results—Enriched Genes are Involved in Multiple Aspects of the Viral Lifecycle and are Broadly Expressed

Upon close examination of the most enriched genes, we found genes involved in key aspects of viral entry and replication (FIG. 2). For example, the well-established entry receptor angiotensin-converting enzyme 2 (ACE2) receptor was ranked as the 12th most-enriched gene in the low MOI screen and 7th in the high MOI screen (See the list in Table I) (Hoffmann et al., 2020a; Zhou et al., 2020). Among the top 50 enriched genes, we identified several sets of related genes that function together in complexes, giving us further confidence in the genome-scale screen (FIGS. 2 and 3A). We found genes essential for initial attachment and endocytosis (ACE2, RAB7A and 4 members of the ARP2/3 complex: ACTR2, ACTR3, ARPC3, and ARPC4), Spike protein cleavage and viral membrane fusion (CTSL and 13 members of the vacuolar-ATPase proton pump: ATP6AP1, ATP6AP2, ATP6V0B ATP6V0C, ATP6V0D1, ATP6V1A, ATP6V1B2, ATP6V1C1, ATP6V1E1, ATP6V1G1, ATP6V1H, TMEM199, and TOR1AIP1), endosome recycling (4 members of the endosomal protein sorting Retromer complex: VPS26A, VPS29, VPS35, and SNX27; 4 members of the endosomal trafficking Commander complex: COMMD2, COMMD3, COMMD3-BMI1, and COMMD4; and 3 members of the PI3K pathway: PIK3C3/VPS34, WDR81, and ACP5), ER-Golgi trafficking (DPM3, ERMP1, PPID, and CHST14), and transcriptional modulators (SLTM and SPEN). A consistent theme among the enriched complexes is endosome function and regulation (V-ATPase proton pump, Retromer, Commander, Class 3 PI3Ks) (Banerjee and Kane, 2020; Mallam and Marcotte, 2017; McNally and Cullen, 2018). Gene Set Enrichment Analysis on the full ranked list of genes identified significantly-enriched Gene Ontology (GO) categories for endosome processing, transport and acidification and categories related to cytokinesis and virion attachment (FDR q<0.1) (FIGS. 3B and 8A-8D; and data not shown) (Subramanian et al., 2005).

Although we performed our CRISPR screen in human lung cells, we explored whether the expression of host genes whose loss reduces SARS-CoV-2 infection were lung-specific or more broadly expressed. To answer this question, we took the top-ranked genes and examined their expression across 12 tissues using 4,790 RNA-sequencing datasets from the Genotype-Tissue Expression (GTEx) v8 database (FIG. 3C) (Aguet et al., 2019). Virtually all of the top gene hits were broadly expressed across all tissues, implying that these mechanisms may function independent of cell or tissue type. Among the top-ranked genes, only ACE2 showed tissue-specific expression with a particular enrichment in testis, small intestine, kidney and heart (FIG. 3C).

Results—Enriched Genes Directly Interact with Viral Proteins and are Also Essential for Other Viral Pathogens

Recently, Krogan and colleagues performed an in-depth study of SARS-CoV-2 protein-protein interaction networks by over-expressing affinity-tagged versions of each protein encoded in the viral genome followed by tandem mass spectrometry after pulldown (Gordon et al., 2020). Their study identified 332 high-confidence SARS-CoV-2-human protein-protein interactions (PPIs). We found that many of the highly-ranked genes from our loss-of-function screen had direct PPI with different viral proteins (FIG. 3D, and data not shown). For example, two highly-ranked subunits of the vacuolar-ATPase proton pump, ATP6AP1 and ATP6V1A, interact with SARS-CoV-2 non-structural protein 6 (nsp6) and membrane (M) protein, respectively. ATP6AP1 was ranked 4th in the low MOI CRISPR screen and 2nd in the high MOI CRISPR screen (See the list of genes in Table I) and its role in viral pathogenesis is further reinforced by its strong PPI interaction (Mass spectrometry Interaction Statistics [MIST] score=0.99) (Verschueren et al., 2015). Another key endocytosis protein, RAB7A, is ranked in the top 50 genes in both CRISPR screens and interacts strongly with non-structural protein 7 (nsp7) (MIST score=0.97). We also compared the top-ranked genes with another proteomic study that used proximity labeling in A549 cells over-expressing BioID-tagged viral proteins and found that 22 out of the top 50 low MOI CRISPR screen genes had direct interactions with viral genes—a significant enrichment over randomly chosen genes (p=2×10−4) (Samavarchi-Tehrani et al., 2020).

Since similar loss-of-function CRISPR screens have been performed to identify host genes required for other viral pathogens, we next sought to understand whether the hits identified in our SARS-CoV-2 screen were shared with those identified in prior screens for ZIKA and pandemic H1N1 avian influenza (IAV) (Y. Li et al. 2019, B. Li et al., 2020). We examined whether top-ranked hits from the ZIKA and IAV screens shared similar genes and similar functional categories. Overall, there was greater similarity between GO categories of enriched genes for SARS-CoV-2 and ZIKA (FIG. 3E) and data not shown. However, when examining the top 50 genes from the SARS-CoV-2 screen, we found several genes that were highly enriched in all 3 viral pathogen screens (FIG. 8E). This group included subunits of the vacuolar-ATPase proton pump, a well-known family of genes essential for acidification and endosomal processing (Banerjee and Kane, 2020).

### Results—Validation of Enriched Genes Using CRISPR Knock-Out, RNA Interference and Small Molecule Inhibitors

To test the ability of top-ranked genes to block SARS-CoV-2 viral infection, we picked 30 genes ranked among the top 200 genes in our RRA analysis for independent validation. Each gene was targeted with 3 guide RNAs distinct from the guides present in the GeCKOv2 genome-wide CRISPR library. The guides were synthesized individually and cloned into the pCC1 lentiviral vector, a modified version of lentiCRISPRv2 with the F+E optimized guide RNA scaffold (Legut et al., 2020).

Cas9-perturbed A549^{ACE2} lines were infected with SARS-CoV-2 at an MOI of 0.1 and the percentage of infected cells was determined by immunofluorescence against the SARS-CoV-2 N protein at 36 hours post-infection (hpi). For all of the Cas9-perturbed cell lines we observed a reduced percentage of infected cells with an up to 10-fold reduction in SARS-CoV-2 infection, compared to the cell lines with non-targeting sgRNAs. Immunofluorescence images of A549ACE2 knockout lines infected with SARS-CoV-2 at MOI of 0.1 and fixed 36 hours post-infection showed SARS-CoV-2 N protein and DAPI (data not shown). Among the genes where loss provided the greatest protection against SARS-2 infection were vesicular trafficking genes like RAB7A, CCDC22 and VPS35, and other genes with well-established roles such as the ACE2 receptor and the protease cathepsin L (CTSL) (FIG. 4A). As an orthogonal gene perturbation method, we also validated a more extensive list of genes via siRNA knockdown for 48 hours, followed by infection with SARS-CoV-2 at MOI of 0.1 (See Table I). Quantification of N-protein immunofluorescent images revealed a substantial reduction of the percent infected cells (FIG. 10A). We found a significant correlation between the percent infection in the arrayed validation and the median fold-change (log 2) from the genome-wide CRISPR screen (rs=−0.6, p=5×10−4) (FIG. 4B).

For these genes, we performed a full multi-step viral replication growth curve and found that the CRISPR perturbations decrease viral load across all time points (5, 10, 24, and 48 h) compared to the non-targeting control (data not shown). To test if the identified hits block viral infection in other cell lines, for 8 genes we generated polyclonal CRISPR knockouts in a human liver cell line (Huh7.5^{ACE2}), which were then infected with SARS-CoV-2 at an MOI of 0.1. Compared to non-targeting guide RNA controls, we found reduced infection with all 8 genes tested in the Huh7.5ACE2 cells ( ).

As an orthogonal gene perturbation method, we also validated a more extensive list of top-ranked genes via siRNA knockdown for 48 h, followed by infection with SARS-CoV-2 at MOI of 0.1. Quantification of N-protein immunofluorescent images revealed a substantial reduction of the percent infected cells (data not shown).

By cross-referencing the top 500 genes from the CRISPR screen with the Drug Gene Interaction database (DGIdb), we identified a set of 69 druggable genes (FIG. 4C; see list of genes in Tables II and III) (Cotto et al., 2018). Based on this list and a manual literature search, we selected 9 genes that were a primary or a secondary target of one of the 26 small molecule inhibitors (FIG. 4D). Among the 26 inhibitors, 9 are FDA approved and 7 are in Phase 2 or Phase 3 clinical trials for diverse ailments. We pretreated A549^{ACE2} cells for 2 hours with 10 μM of each inhibitor and then infected with SARS-CoV-2 and analyzed the cells at 36 hpi. As a positive control, we also included remdesivir, which inhibits the viral RNA polymerase and is the only currently approved treatment for COVID-19 in the United States (Beigel et al., 2020). We assessed the efficacy of each inhibitor on blocking viral infection using immunofluorescence and quantitative PCR (qPCR).

Seven of the 26 inhibitors that we tested (PIK-III, Compound-19, SAR405, autophinib, ALLN, tamoxifen and ilomastat) resulted in >100-fold reduction of viral load as measured by qPCR (FIG. 4D). Among the best performing inhibitors, 4 of them target the same gene VPS34 and 2 inhibitors (autophinib and ALLN) reduce the viral load more than 1000-fold. Similar results were obtained by immunofluorescent imaging of SARS-CoV-2 N protein (FIG. 10B). By testing the top four PIK3C3 inhibitors in combination with CRISPR targeting of PIK3C3, we found that Compound-19, PIK-III and autophinib were specific while SAR405 resulted in greater viral inhibition in PIK3C3 CRISPR-perturbed cells, suggesting some potential off-target activity for SAR405 (FIG. 10, and data not shown).

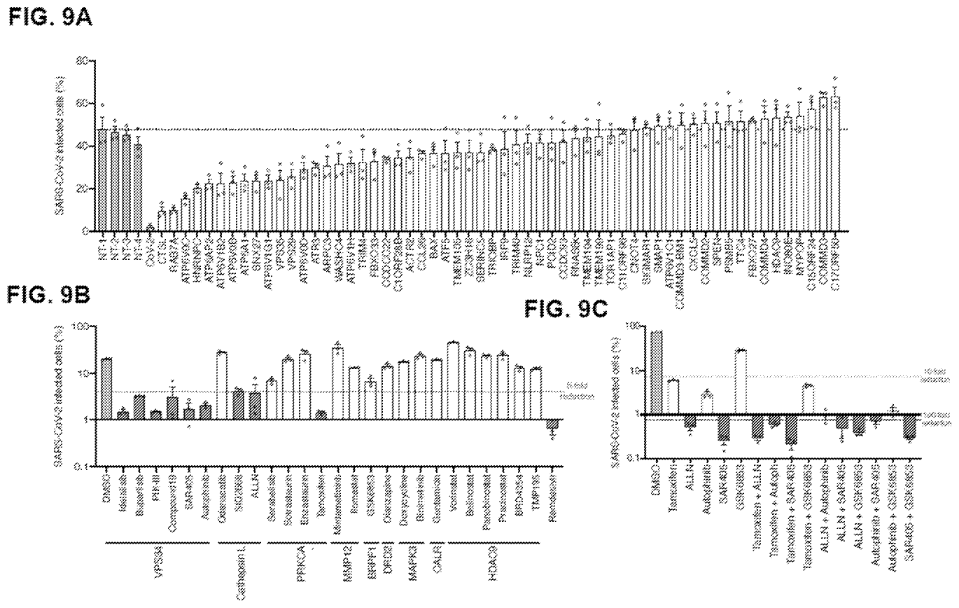

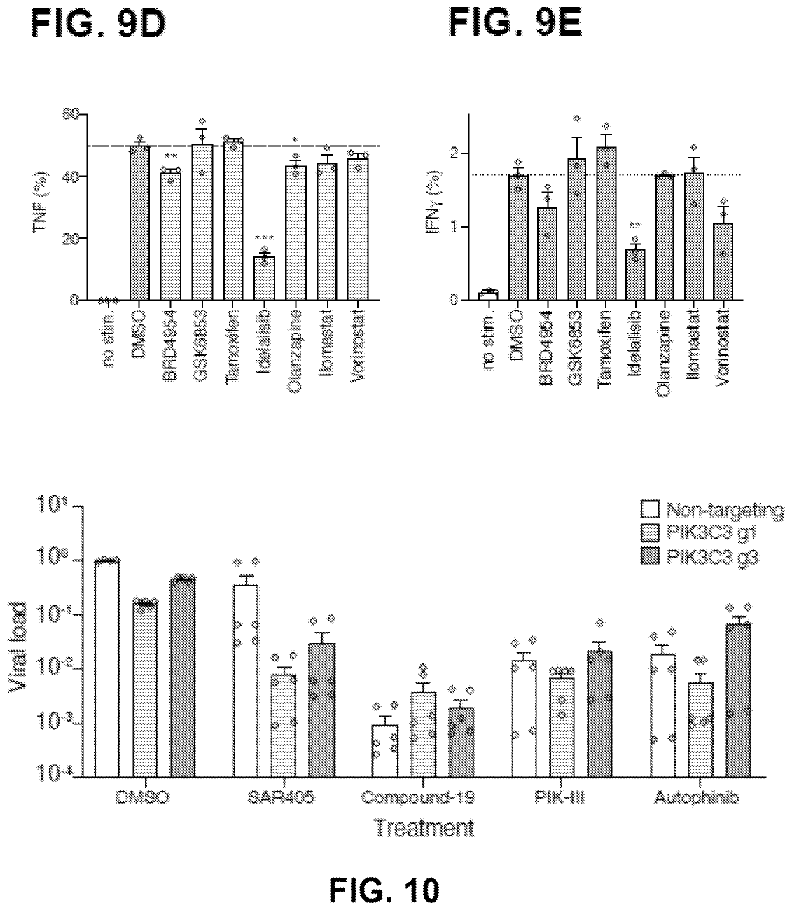

Combinations of the top performing inhibitors overall showed an additive effect and further protected the cells from SARS-2 infection, with the best protection observed by combining tamoxifen and SAR405 (FIG. 10C). Next, we measured cell viability at 36 hours post inhibitor treatment using flow cytometry. We observed more than 50% reduction of A549^{ACE2} cell viability with two pan-HDAC inhibitors, panobinostat and pracinostat (FIG. 4F). To explore the effects of the inhibitors on other important cell types during viral infection, we tested the effect of select inhibitors on primary human CD8+ T-cell activation. Human primary CD8+ T-cell gating strategy was as follows. Cells were first gated by the forward and side scatter area and then doublets were excluded by gating through the forward scatter area and width. Live CD8+ cells were selected by gating on LIVE-DEAD violet and CD8+ FITC. The non-stimulated or CD3/CD28 stimulated viable CD8+ T cells were gated on TNF-a PE/Cy7 and IFNg PE to determine the percentage of TNF-α and IFNg positive cells. (data not shown). Among the tested inhibitors, only idelalisib resulted in a significant reduction in release of cytokines tumor necrosis factor (TNF) and interferon-gamma (IFNγ), consistent with previous reports (FIG. 9D, 9E) (Martinelli et al., 2018). Other inhibitors with higher activity in blocking SARS-CoV-2 infection (e.g. tamoxifen) had no effect on T-cell activation and cytokine secretion (FIG. 9C, 10D-10E).

Results—Single-Cell Sequencing Identifies Cholesterol Biosynthesis as a Common Mechanism Underlying Multiple Enriched Genes from the CRISPR Screen

Next, we sought to understand the mechanisms underlying how individual genes identified in our loss-of-function screen prevent SARS-CoV-2 infection and if host gene loss alters cell transcriptional programs. For this, we utilized the Expanded CRISPR-compatible Cellular Indexing of Transcriptomes and Epitopes by sequencing (ECCITE-seq) method to couple pooled CRISPR perturbations of our top hit genes with a single-cell transcriptomic and proteomic readout (Mimitou et al., 2019) (FIG. 5A; and data not shown). Importantly, cells were infected at low MOI to maximize the fraction of cells that express a single guide RNA, and therefore can be assigned a specific gene perturbation. For this, we pooled all 90 individual guide RNA plasmids (3 per target gene) used to validate our genome-scale screen and 9 non-targeting (NT) sgRNAs (FIG. 4A).

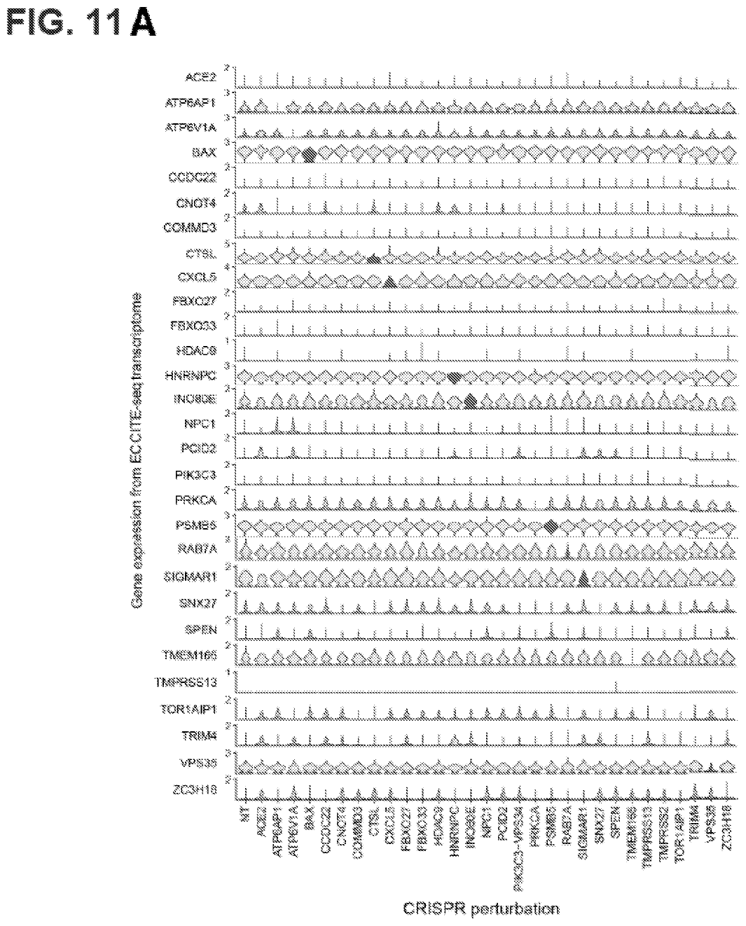

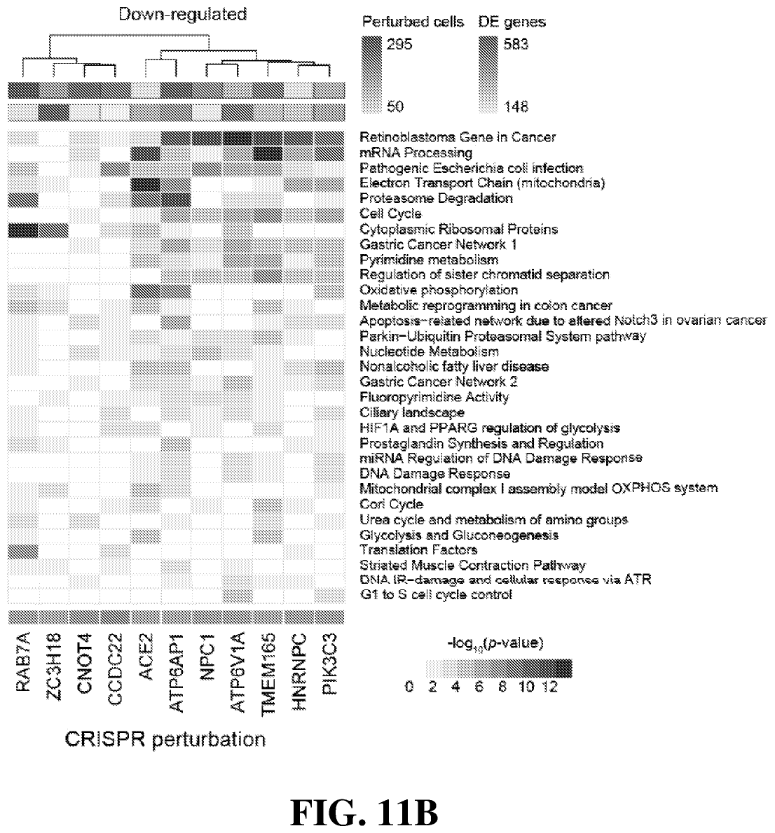

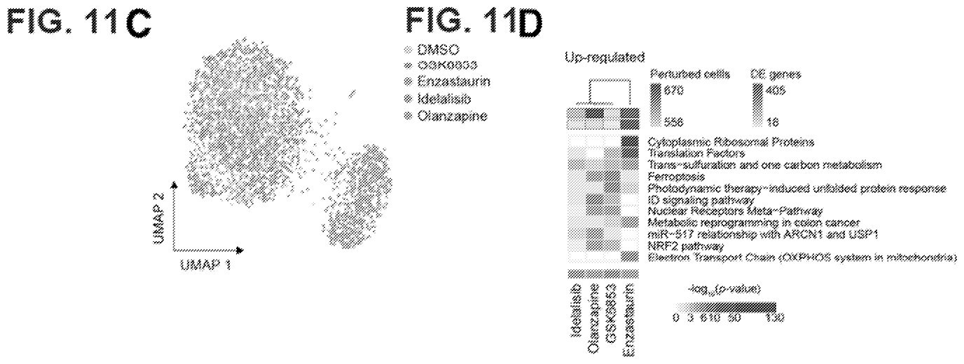

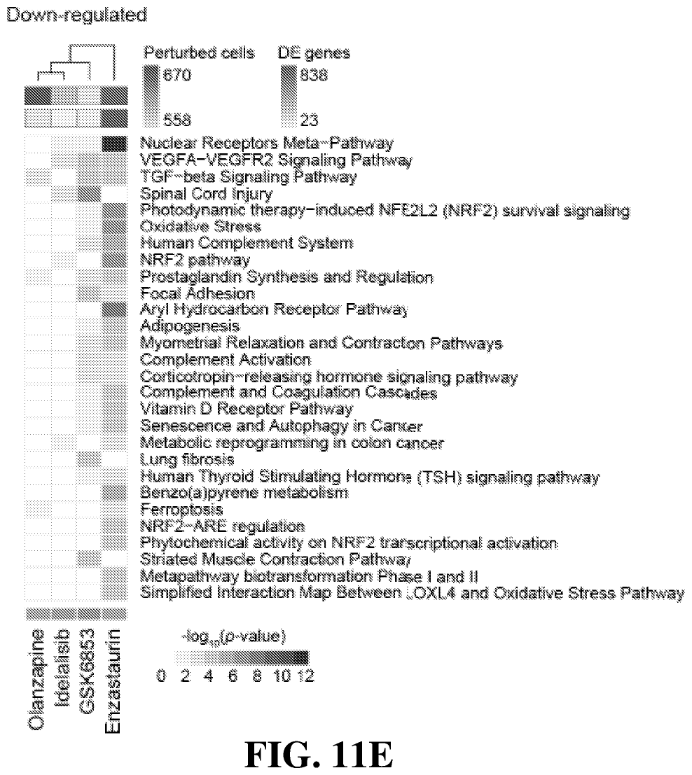

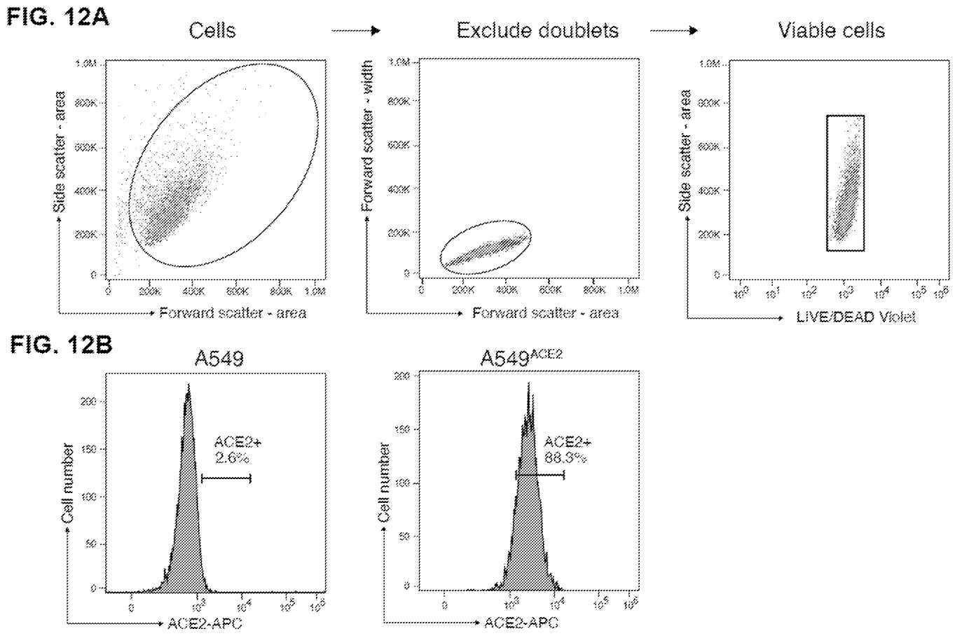

Using ECCITE-seq, we identified a median of 152 single cells per target gene with a single guide RNA. We observed specific reduction of target gene expression in cells grouped by target gene, indicating nonsense-mediated decay of transcripts with frameshift indel mutations after CRISPR modification (FIG. 11A). This effect was more pronounced for genes with higher expression. To classify cells for each target gene as either ‘perturbed’ (different from cells with NT guide RNAs) or ‘escaping’ (similar to cells with NT sgRNAs), we used a recently described Gaussian mixture model framework (Papalexi et al., 2020). Using this framework, we classified cells for 11 of the 30 target genes more than 5 differentially expressed “perturbed” genes with a minimal log fold change (FIG. 5B), implying that loss of these genes results in a detectable transcriptomic shift. It is likely that loss of the other 19 genes results in more subtle changes in only a few genes; however, even for ‘non-perturbed’ genes with sufficient basal expression, we detected clear changes in the CRISPR target gene (FIG. 11A). We repeated the ECCITE-seq experiment focusing on the 11 genes with detectable transcriptomic shifts upon target gene perturbation. Combining both replicate experiments, we obtained 18,853 cells that expressed only one guide RNA with a median of 1,388 cells per target gene.

We found that loss of 6 of the ‘perturbed’ genes—ATP6AP1, ATP6V1A, CCDC22, NPC1, PIK3C3 and RAB7A, which are part of the endosomal entry pathway—yielded similar gene expression signatures among upregulated differentially expressed genes (FIGS. 5C, 5D and 11B). These 6 target gene perturbations all led to upregulation of pathways affecting lipid and cholesterol homeostasis (FIG. 5D). Recently, we performed a large survey of >20,000 potential pharmacological treatments for COVID-19 and, for compounds effective at preventing viral infection, we identified induction of the cholesterol biosynthesis pathway as a potential mechanism of viral inhibition (Hoagland et al., 2020). Loss-of-function mutations in these 6 genes may function through a similar mechanism (induction of cholesterol synthesis) that combats the virus-mediated suppression of cholesterol synthesis. Among the significant differentially-expressed genes, we also found 61 genes from the enriched CRISPR screen genes (n=20 genes upregulated; n=41 genes down-regulated). For example, NPC1, ATP6V1F and ATP6V1E1 are upregulated in most of the 6 endosomal entry pathway gene-perturbed cells (FIG. 5C), suggesting compensatory upregulation of related genes to mitigate target gene loss.

To understand how these changes impact lipid production, we measured cholesterol levels in cells after CRISPR perturbation and found that loss of these genes increases cholesterol by between 10% and 50%, depending on the perturbation (FIG. 5D). To show that increases in cholesterol leads to increased SARS-CoV-2 resistance, we treated A549^{ACE2} cells with amlodipine, a calcium-channel antagonist that increases intracellular cholesterol (Mori et al., 1988; Ranganathan et al., 1982). We verified that amlodipine increases cholesterol levels in A549^{ACE2} cells (data not shown) and found that pre-treatment with amlodipine results in reduced SARS-CoV-2 viral infection, as measured by qPCR for nucleocapsid RNA, plaque formation, and number of viral RNA reads from RNA-sequencing, with only a modest impact on cell viability (data not shown). RNA-sequencing of cells treated with amlodipine shows a similar differential gene expression profile as seen in our ECCITE-seq with CRISPR perturbations with the most significant upregulated pathway as cholesterol biosynthesis (data not shown).

### Results—RAB7A Knockout Results in a Reduced Cell Surface Expression and an Increased Endosomal Accumulation of ACE2

Next, we sought to determine if any of the top-ranked genes regulate cell surface expression of ACE2, as surface ACE2 is required for SARS-CoV-2 infection (Hoffmann et al., 2020a). To measure the cell surface expression of ACE2, we performed flow cytometry on A549 wild-type cells and a panel of A549^{ACE2} CRISPR-perturbed cells. ACE2 expression was detected in A549^{ACE2}, but not in A549 wild type cells, validating the antibody specificity (FIGS. 6A and 12A-12C). Flow cytometry analysis of the A549^{ACE2} CRISPR-perturbed lines revealed that RAB7A knock-out cells have a significantly reduced cell surface expression of ACE2 compared to cells transduced with a non-targeting guide RNA (FIG. 6B). Rab7a is a small GTPase that is involved in regulating cellular processes such as vesicular transport and membrane trafficking (Guerra and Bucci, 2016). We hypothesized that the observed reduction of ACE2 at the cell surface in RAB7A knock-out cells may be due to impaired vesicular trafficking and accumulation of ACE2 in the cytoplasm.

Immunofluorescence staining of ACE2 on the A549^{ACE2} polyclonal cell line was targeted with a non-targeting (NT) or a RAB7A-targeting guide. In NT cells, ACE2 localizes at the cell membrane and in the cytoplasm, while in RAB7A-targeted cells, ACE2 shows a distinct pattern of localization to vesicles. ACE2 is detected in most cells, and RAB7A knockout leads to revealed an increased accumulation of ACE2 in the cytoplasm and in vesicle-like hollow structures reminiscent of endo-lysosomes (data not shown). We detected these accumulations in about 35% of RAB7A knockout cells with ACE2 staining, compared to the control where ACE2 was primarily localized at the plasma membrane and small cytoplasmic puncta (FIG. 6C). Finally, we investigated which cellular compartments accumulate ACE2 in RAB7A knockout cells. Co-immunofluorescence images showed that in RAB7A knockout cells ACE2-containing vesicles often colocalize with EEA1, an early endosomal marker and less frequently with LysoTracker, a lysosome marker (data not shown). Efficient Rab7a protein depletion across the RAB7A CRISPR-perturbed lines used was confirmed by western blot (FIG. 12C).

Since ACE2 was overexpressed in our A549^{ACE2} cells, we considered whether Rab7A loss would lead to similar cytoplasmic sequestration in cells with endogenous ACE2 expression. Flow cytometry analysis in Caco-2 colon and Calu-3 lung cells revealed that RAB7A knock-out cells have significantly reduced cell surface expression of ACE2 compared to cells transduced with a non-targeting guide RNA (FIG. 6D, 6E). Immunofluorescence staining of ACE2 on Caco-2 cells transduced with a NT or a RAB7A-targeting guide showed that in NT cells, ACE2 localizes at the cell membrane and in the cytoplasm, while in RAB7A-targeted cells, ACE2 shows a distinct pattern of localization to vesicles. We also found that RAB7A knock-out in Caco-2 cells results in larger cytoplasmic ACE2 puncta compared to the control cells, further supporting cytoplasmic accumulation of ACE2 in the absence of Rab7A (FIG. 6F).

### Results—Genes Required for SARS-CoV-2 Infection In Vitro Contribute to COVID-19 Risk in Human Patients

Quantile-quantile (Q-Q) plots showed the expected and observed p-value distribution of COVID-19 hospitalized versus not hospitalized genome-wide association study (GWAS) for three sets of variants: all variants tested in the GWAS, variants that are lead eQTLs in GTEx Lung, and variants that are lead eQTLs in GTEx Lung for the top-ranked genes enriched in at least one CRISPR screen (data not shown). Inflation estimate λ_{0.1} measures the inflation of test statistics relative to the χ² quantile function of 0.1. i.e., 10% of the most significant tests. A histogram of the permuted λ_{0.1} estimate to test the significance of the inflation of the signal for variants that are eQTLs for CRISPR screen hit genes (n=243 variants) in Lung in the COVID-19 hospitalized versus not hospitalized GWAS was performed. The observed λ_{0.1} value was 2.14 (data not shown). Prioritization of genes in the 3p21.31 locus associated with COVID-19 versus population GWAS was studied. Genes in the locus were ranked according to the second-best guide RNA score in the low MOI and high MOI pooled CRISPR screens. eQTL p-values for the lead GWAS variant 3:45867022:C:G was shown in different cell types and tissues from the eQTL Catalogue and GTEx v8, 110 eQTL data sets in total. Nine cell types/tissues were highlighted, where the eQTL p-value for the lead GWAS variant is <10⁻⁴ for at least one gene in the region. Suggestive support for colocalization between the GWAS and eQTL signal (posterior probability for one shared causal variant (PP4)>0.25 was shown. The transcripts of the eight protein-coding genes (LIMD1, SACM1L, SLC6A20, LZTFL1, CCR9, FYCO1, CXCR6 and XCR1) in the locus were obtained. Ranks and eQTL p-values were aligned to match the start of the. The regional association of the COVID-19 versus population GWAS and cis-eQTLs for the eight genes from the associated locus in the cell type/tissue where the lead GWAS variant 3:45867022:C:G has the lowest eQTL p-value was shown. All above data was shown in the provisional application but not shown here.

Lastly, we sought to determine to what extent top-ranked genes from the CRISPR screen mediate COVID-19 risk. Genetic variants have been shown to often exert their effect on complex traits or disease via cis-regulation of gene expression (Gamazon et al., 2018). Thus, we hypothesized that genetic regulatory variants for genes pinpointed by the CRISPR screen would show an increased signal for genetic association for COVID-19 in the human population. To this end, we analyzed if expression quantitative trait loci (eQTLs) from lung tissue for these genes would be enriched for overall association signal in data from the COVID-19 Host Genetic Initiative (HGI) with six COVID-19-related genome-wide association studies (GWAS) (Aguet et al., 2019; COVID-19 Host Genetics Initiative, 2020). The Q-Q plots compared the expected and observed P-value distribution of COVID-19 GWAS P-value (−log 10) for all the variants tested in GWAS, variants that are lead eQTLs in GTEx Lung, variants that are lead eQTLs in GTEx Lung for the top-ranked genes enriched in at least one CRISPR screen for ANA_A2_V2 (very severe respiratory confirmed COVID-19 vs. population), ANA_B2_V2 (hospitalized COVID-19 vs. population), ANA_C1_V2 (COVID-19 vs. lab/self reported negative), ANA_C2_V2 (COVID-19 vs. population), and ANA_D1_V2 (predicted COVID-19 from self-reported symptoms vs. predicted or self-reported non-COVID-19) GWAS, respectively. Inflation estimate λ0.1 measures the inflation of test statistics relative to the chi-square quantile function of 0.1. i.e., 10% of the most significant tests (data not shown) The histogram of the permuted 70.1 estimate was observed to test the significance of the inflation of variants that are eQTLs for CRISPR screen hit genes in GTEx Lung in the given COVID-19 GWAS (data not shown). We observed inflation of GWAS association signal for variants that are cis-eQTLs for top-ranked CRISPR genes in the hospitalized versus not hospitalized COVID-19 GWAS, compared to all lung eQTLs (λ_{0.1}=2.14, permutation p-value=0.03,). We did not see a significant signal for the other five GWASs, which could be explained by the low power of these early GWAS data (data not shown). Nevertheless, our results suggest that genes that are required for SARS-CoV-2 infection in vitro contribute to COVID-19 risk in humans.

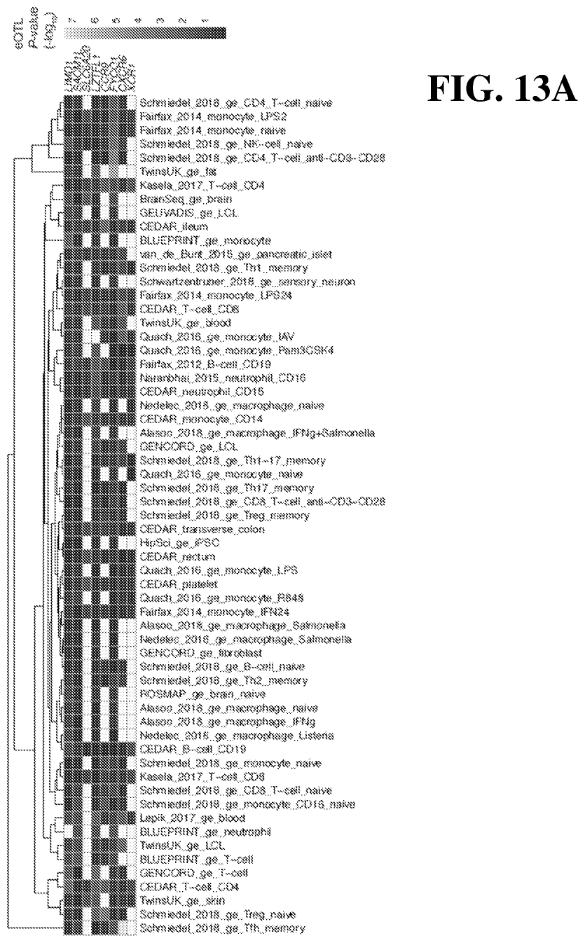

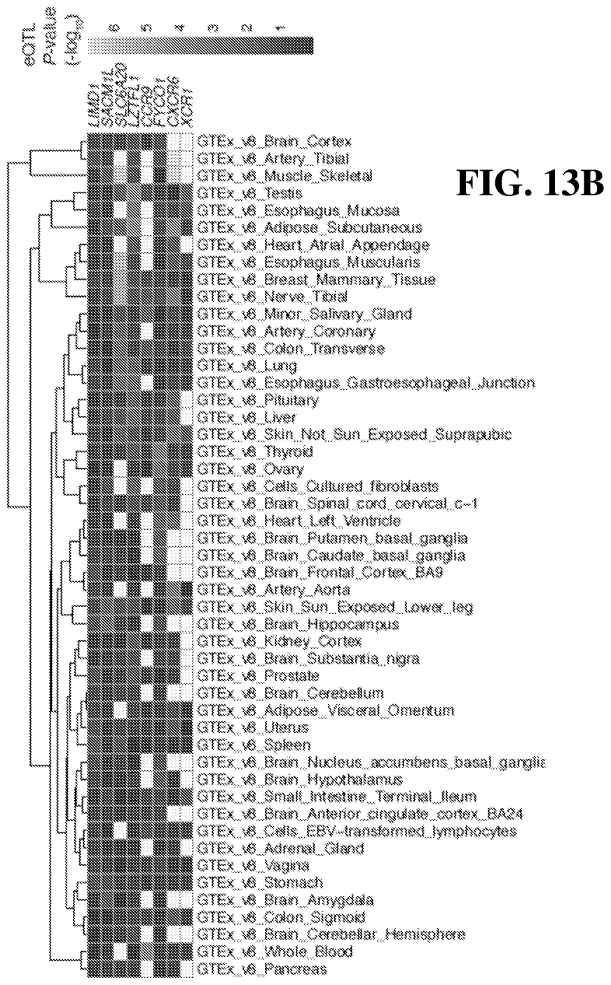

The only individual locus with a robust human genetic association to COVID-19 susceptibility is 3p21.31, but the functional mechanisms of this association have been unclear and the locus includes multiple protein-coding genes (for example, LIMD1, SACM1L, SLC6A20, LZTFL1, CCR9, FYCO1, CXCR6, and XCR1) (COVID-19 Host Genetics Initiative, 2020; Ellinghaus et al., 2020). To elucidate which genes might mediate the genetic association, we first observed that the SLC6A20 gene and the CXCR6 gene have a relatively high rank in our CRISPR screen (data not shown). Next, we analyzed if the COVID-19 ANA_C2_V2 GWAS lead variant 3:45867022:C:G affects the expression of any of these genes. Using data from the eQTL Catalogue and GTEx v8 (Aguet et al., 2019; Kerimov et al., 2020), we analyzed all eQTL associations for this variant, and performed colocalization analysis to assess if the eQTL and GWAS signal share the same causal variant. CXCR6 and SLC6A20 stood out, with the eQTL data indicating that the COVID-19-associating variant affects the expression of these two genes (FIGS. 13A, 13B) (Giambartolomei et al., 2014; Wallace, 2020). The eQTL affecting CXCR6 is active in memory T follicular helper (Tfh) CD4⁺ T cells and has a suggestive colocalization with the GWAS (data not shown) (Schmiedel et al., 2018). The colocalization signal is very strong for the cis-eQTL for SLC6A20 in four tissues from GTEx. Colocalization analysis of COVID-19 GWAS and eQTLs was performed in different cell types and tissues. Posterior probability (y-axis) for the five different hypothesis H0 (no association), H1 (association in GWAS), H2 (association in eQTL), H3 (association both in GWAS and eQTL, two independent SNPs), H4 (association both in GWAS and eQTL, one shared SNP) from coloc is shown for each of the eight genes (LIMD1, SACM1L, SLC6A20, LZTFL1, CCR9, FYCO1, CXCr6, and XCR1) used in the colocalization analysis, with results from the eQTL Catalogue and GTEx v8. We used the ANA-C2-V2 (COVID-19 vs. population) GWAS. For eQTL studies using microarray technology with multiple probes mapping to the same gene, we chose the probe which resulted in the lowest eQTL p-value for the lead COVID-19 GWAS variant to represent the gene. While we observe suggestive signal for colocalization with eQTLs for CXCR6 in Tfh memory cells (Schmiedel et al. 2018), most posterior support is attributed to H1 indicating insufficient power. Also, there is suggestive signal for colocalization with eQTLs for LZTFL1 in Testis in GTEx, but the eQTL signal is not statistically significant (FDR >0.05) and lead eQTL variant has GWAS p-value=0.4011. (data not shown). Other genes in the region, such as LZTFL1, FYCO1, and XCR1, also have regulatory variants affecting their expression, but two distinct variants likely drive the GWAS and eQTL signals (data not shown). Given that both the CRISPR screen and eQTL data support the causal role of SLC6A20 and CXCR6, it is possible that the COVID-19 GWAS association in the 3p21.31 locus is driven by pleiotropic effects of the same variant on multiple genes in different cell types.

Each and every patent, patent application, and publication, including websites cited throughout specification, are incorporated herein by reference. Similarly, the SEQ ID NOs which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

### REFERENCES

1000 Genomes Project Consortium, Auton, A., et al. (2015). A global reference for human genetic variation. Nature 526, 68-74.
Aguet, F., et al. (2019). The GTEx Consortium atlas of genetic regulatory effects across human tissues. BioRxiv 787903.
Aizaki, H., et al. (2008). Critical role of virion-associated cholesterol and sphingolipid in hepatitis C virus infection. J. Virol. 82, 5715-5724.
Bajimaya, S., et al. (2017). Cholesterol is required for stability and infectivity of influenza A and respiratory syncytial viruses. Virology 510, 234-241.
Banerjee, S., and Kane, P. M. (2020). Regulation of V-ATPase Activity and Organelle pH by Phosphatidylinositol Phosphate Lipids. Front Cell Dev Biol 8, 510.
Beigel, J. H., et al. (2020). Remdesivir for the Treatment of Covid-19-Preliminary Report. N. Engl. J. Med.
Blanco-Melo, D., et al. (2020). Imbalanced Host Response to SARS-CoV-2 Drives Development of COVID-19. Cell 181, 1036-1045.e9.
Bouhaddou, M., et al. (2020). The Global Phosphorylation Landscape of SARS-CoV-2 Infection. Cell 182, 685-712.e19.
Chang, T.-Y., et al. (2005). Niemann-Pick type C disease and intracellular cholesterol trafficking. J. Biol. Chem. 280, 20917-20920.
Chen, C.-H., et al. (2018). Improved design and analysis of CRISPR knockout screens. Bioinformatics 34, 4095-4101.
Chen, S., et al. (2015). Genome-wide CRISPR screen in a mouse model of tumor growth and metastasis. Cell 160, 1246-1260.
Cotto, K. C., et al. (2018). DGIdb 3.0: a redesign and expansion of the drug-gene interaction database. Nucleic Acids Res. 46, D1068-D1073.
COVID-19 Host Genetics Initiative (2020). The COVID-19 Host Genetics Initiative, a global initiative to elucidate the role of host genetic factors in susceptibility and severity of the SARS-CoV-2 virus pandemic. Eur. J. Hum. Genet. 28, 715-718.
Daniloski, Z., et al. (2020). The Spike D614G mutation increases SARS-CoV-2 infection of multiple human cell types. BioRxiv 2020.06.14.151357.
Daniloski, Z. et al., (2021 January). Identification of Required Host Factors for SARS-CoV-2 Infection in Human Cells. Cell, 184(1): 92-105.e16 (published online Oct. 24, 2020)
Eden, E., et al. (2009). GOrilla: a tool for discovery and visualization of enriched GO terms in ranked gene lists. BMC Bioinformatics 10, 48.
Ellinghaus, D., et al. (2020). Genomewide Association Study of Severe Covid-19 with Respiratory Failure. N. Engl. J. Med.
ENCODE Project Consortium (2012). An integrated encyclopedia of DNA elements in the human genome. Nature 489, 57-74.
Erb, M. A., et al. (2017). Transcription control by the ENL YEATS domain in acute leukaemia. Nature 543, 270-274.
Funk, C. D., et al. (2020). A Snapshot of the Global Race for Vaccines Targeting SARS-CoV-2 and the COVID-19 Pandemic. Front Pharmacol 11, 937.
Gamazon, E. R., et al. (2018). Using an atlas of gene regulation across 44 human tissues to inform complex disease- and trait-associated variation. Nat. Genet. 50, 956-967.
Gardner, L. (2020). Johns Hopkins Coronavirus Resource Center.
Gates, B. (2020). Responding to Covid-19-A Once-in-a-Century Pandemic? N. Engl. J. Med. 382, 1677-1679.
Giambartolomei, C., et al. (2014). Bayesian test for colocalisation between pairs of genetic association studies using summary statistics. PLOS Genet. 10, e1004383.
Girard, E., et al. (2014). Rab7 is functionally required for selective cargo sorting at the early endosome. Traffic 15, 309-326.
Gordon, D. E., et al. (2020). A SARS-CoV-2 protein interaction map reveals targets for drug repurposing. Nature 583, 459-468.
Guerra, F., and Bucci, C. (2016). Multiple Roles of the Small GTPase Rab7. Cells 5.
Hoagland, D. A., et al. (2020). Modulating the transcriptional landscape of SARS-CoV-2 as an effective method for developing antiviral compounds. BioRxiv 2020.07.12.199687.
Hoffmann, M et al. (2020a). SARS-CoV-2 Cell Entry Depends on ACE2 and TMPRSS2 and Is Blocked by a Clinically Proven Protease Inhibitor. Cell 181, 271-280.e8.
Hoffmann, M., et al. (2020b). A Multibasic Cleavage Site in the Spike Protein of SARS-CoV-2 Is Essential for Infection of Human Lung Cells. Mol. Cell 78, 779-784.e5.
Jain, I. H., et al. (2016). Hypoxia as a therapy for mitochondrial disease. Science 352, 54-61.
Kerimov, N., et al. (2020). eQTL Catalogue: a compendium of uniformly processed human gene expression and splicing QTLs. BioRxiv 2020.01.29.924266.
Kim, D., et al. (2020). The Architecture of SARS-CoV-2 Transcriptome. Cell.
Kolde, R., et al. (2012). Robust rank aggregation for gene list integration and meta-analysis. Bioinformatics 28, 573-580.
Korotkevich, G., et al. (2019). Fast gene set enrichment analysis. BioRxiv 060012.
Kuleshov, M. V., et al. (2016). Enrichr: a comprehensive gene set enrichment analysis web server 2016 update. Nucleic Acids Res. 44, W90-97.
Legut, M., et al. (2020). High-throughput screens of PAM-flexible Cas9 variants for gene knock-out and transcriptional modulation. Cell Reports 2020.01.22.916064.
Lek, A., et al. (2020). Applying genome-wide CRISPR-Cas9 screens for therapeutic discovery in facioscapulohumeral muscular dystrophy. Sci Transl Med 12.
Li, B., et al. (2020). Genome-wide CRISPR screen identifies host dependency factors for influenza A virus infection. Nat Commun 11, 164.
Li, Y., (2019). Genome-wide CRISPR screen for Zika virus resistance in human neural cells. Proc. Natl. Acad. Sci. U.S.A. 116, 9527-9532.
Liu, Y., (2020). The reproductive number of COVID-19 is higher compared to SARS coronavirus. J Travel Med 27.
Luo, B., et al. (2008). Highly parallel identification of essential genes in cancer cells. Proc. Natl. Acad. Sci. U.S.A. 105, 20380-20385.
Mallam, A. L., and Marcotte, E. M. (2017). Systems-wide Studies Uncover Commander, a Multiprotein Complex Essential to Human Development. Cell Syst 4, 483-494.
Marchant, J. (1976). Letter: Prophylactic chemotherapy for breast cancer. Br Med J 1, 1589.
Martinelli, S., et al. (2018). Idelalisib impairs T-cell-mediated immunity in chronic lymphocytic leukemia. Haematologica 103, e598-e601.
McNally, K. E., and Cullen, P. J. (2018). Endosomal Retrieval of Cargo: Retromer Is Not Alone. Trends Cell Biol. 28, 807-822.
Meier, J. A. et al. (2017). GUIDES: sgRNA design for loss-of-function screens. Nat. Methods 14, 831-832.
Millard, E. E., et al. (2000). Niemann-pick type C1 (NPC1) overexpression alters cellular cholesterol homeostasis. J. Biol. Chem. 275, 38445-38451.
Mimitou, E. P., et al. (2019). Multiplexed detection of proteins, transcriptomes, clonotypes and CRISPR perturbations in single cells. Nat. Methods 16, 409-412.
Neufeld, E. B., et al. (1996). Intracellular trafficking of cholesterol monitored with a cyclodextrin. J. Biol. Chem. 271, 21604-21613.
O'Brian, C. A., et al (1985). Inhibition of protein kinase C by tamoxifen. Cancer Res. 45, 2462-2465.
Papalexi, E., et al. (2020). Characterizing the molecular regulation of inhibitory immune checkpoints with multi-modal single-cell screens. BioRxiv 2020.06.28.175596.
Patel, S. J., et al. (2017). Identification of essential genes for cancer immunotherapy. Nature 548, 537-542.
Riva, L., et al. (2020). Discovery of SARS-CoV-2 antiviral drugs through large-scale compound repurposing. Nature.
Rush, J. S., and Ceresa, B. P. (2013). RAB7 and TSG101 are required for the constitutive recycling of unliganded EGFRs via distinct mechanisms. Mol. Cell. Endocrinol. 381, 188-197.
Sanjana, N. E., et al. (2014). Improved vectors and genome-wide libraries for CRISPR screening. Nat. Methods 11, 783-784.
Sanjana, N. E., et al. (2016). High-resolution interrogation of functional elements in the noncoding genome. Science 353, 1545-1549.
Schmiedel, B. J., et al. (2018). Impact of Genetic Polymorphisms on Human Immune Cell Gene Expression. Cell 175, 1701-1715.e16.
Shalem, O., et al. (2014). Genome-scale CRISPR-Cas9 knockout screening in human cells. Science 343, 84-87.
Slenter, D. N., et al. (2018). WikiPathways: a multifaceted pathway database bridging metabolomics to other omics research. Nucleic Acids Res. 46, D661-D667.
Stoeckius, M., et al. (2018). Cell Hashing with barcoded antibodies enables multiplexing and doublet detection for single cell genomics. Genome Biol. 19, 224.
Stuart, T., et al. (2019). Comprehensive Integration of Single-Cell Data. Cell 177, 1888-1902.e21.
Subramanian, A., et al. (2005). Gene set enrichment analysis: a knowledge-based approach for interpreting genome-wide expression profiles. Proc. Natl. Acad. Sci. U.S.A. 102, 15545-15550.
Uhlen, M., et al. (2010). Towards a knowledge-based Human Protein Atlas. Nat. Biotechnol. 28, 1248-1250.
Verschueren, E., et al. (2015). Scoring Large-Scale Affinity Purification Mass Spectrometry Datasets with MiST. Curr Protoc Bioinformatics 49, 8.19.1-8.19.16.
Visel, A., et al. (2009). Genomic views of distant-acting enhancers. Nature 461, 199-205.
Wallace, C. (2020). Eliciting priors and relaxing the single causal variant assumption in colocalisation analyses. PLOS Genet. 16, e1008720.
Zhou, P., et al. (2020). A pneumonia outbreak associated with a new coronavirus of probable bat origin. Nature 579, 270-273.

1. A method of treating or inhibiting a viral infection in a human subject comprising inhibiting in vivo the expression or activity of one or a combination of the subject's genes required for viral infection by administering to a mammalian subject in need thereof one or more inhibitors of the activity or expression of said gene or combination of genes.

2. The method according to claim 1,
wherein the viral infection is COVID-19,
wherein the gene or combination of genes comprises one or more of PIK3C3, PRKCA, Cathepsin L, MMP12 and ACE2, and
wherein:
(i) the inhibitor of PIK3C3 is one or more of Autophinib, SAR405, Compound 19, or PIK-III;
(ii) the inhibitor of PRKCA is Tamoxifen;
(iii) the inhibitor of Cathepsin L is ALLN, CAS No. 110044-82-1;
(iv) the inhibitor of MMP12 is Ilomastat; and
(v) the inhibitor of ACE2 is one or more of Compound 28 (PMID: 18324760), XNT (PMID: 18391097), MLN-4760, GL11001, and ABZ-Ser-Pro-Tyr (NO₂)—OH, and Lisinopril.

3. The method according to claim 1, wherein said gene or combination of genes is selected from the genes identified in Tables I, II, III or IV.

4. The method according to claim 1, wherein said inhibitor is selected from one or a combination of the inhibitors identified in Table III or Table IV.

5. The method according to claim 1, wherein said inhibitor is selected from one or a combination of the inhibitors identified in FIG. 4D or 4E.

6. The method according to claim 1, wherein said inhibitor is one or a combination of Remdesivir, PIK-III, Compound 19, SAR405, Autophinib, ALLN, Tamoxifen and Ilomastat.

7. The method according to claim 1, wherein the selected inhibitor is administered to the subject pre-infection or post-infection at a dosage effective to mimic a loss of function of its corresponding gene.

8. The method according to claim 1, wherein said gene is RAB7A, ACE2, ACTR2, ACTR3, ARPC3, and ARPC4.

9. The method according to claim 1, wherein said gene or combination comprises one or more of CTSL, ATP6AP1, ATP6AP2, ATP6V0B ATP6V0C, ATP6V0D1, ATP6V1A, ATP6V1B2, ATP6V1C1, ATP6V1E1, ATP6V1G1, ATP6V1H, TMEM199, and TOR1AIP1.

10. The method according to claim 1, wherein said gene or combination comprises one or more of VPS26A, VPS29, VPS35, and SNX27.

11. The method according to claim 1, wherein said gene or combination comprises one or more of COMMD2, COMMD3, COMMD3-BMI1, and COMMD4.

12. The method according to claim 1, wherein said gene or combination comprises one or more of PIK3C3/VPS34, WDR81, and ACP5.

13. The method according to claim 1, wherein said gene or combination comprises one or more of DPM3, ERMP1, PPID, CHST14, SLTM and SPEN.

14. The method according to claim 1, wherein said gene or combination comprises one or more of PIK3C3, Cathepsin L, PRKCA, MMP12, BRPF1, DRD2, MAPK3, CALR and HCAC9.

15. The method according to claim 1, wherein the genes are one or more of genes or subsets of genes identified in FIG. 1D, FIG. 2, FIG. 3C, FIG. 4A, FIG. 4D, FIG. 5C, FIG. 6B, or FIG. 8E.

16.-21. (canceled)

22. The method according to claim 1, wherein the genes are one or more of the 69 genes identified in Table III.

23. The method according to claim 1, wherein the genes are one or more of the 73 genes identified in Table IV.

24. The method according to claim 1, wherein the viral infection is caused by infection with the virus SARS-CoV-2.

25. The method according to claim 1, wherein the inhibitor comprises a small molecule, a nucleic acid therapeutic, a peptide therapeutic, or another biologic therapeutic that mimics in the subject a loss of function of one or more of the selected genes.

26. A method for identifying host target genes required for viral infection targeting lung cells comprising performing a genome-scale loss of function screen by
a) transducing a selected cell line with a lentiviral vector containing Cas9, guide RNA from the GeCKOv2 human library, and an antibiotic resistance gene at a low MOI to cause CRISPR gene targeting;
b) culturing the cells to ensure protein depletion after CRISPR gene targeting using said guide RNA;
c) infecting pooled cells of (b) with selected virus;
d) extracting genomic DNA and, via amplicon sequencing, quantifying guide abundance in each biological condition;
e) identifying genes where loss-of-function mutations led to enrichment within the pools.


SEQUENCE LISTING (9 sequences)

SEQ ID NO: 1 - DNA, 22 bp, Artificial Sequence
  Primer
gagggcctatttcccatgattc

SEQ ID NO: 2 - DNA, 23 bp, Artificial Sequence
  Primer
gttgcgaaaaagaacgttcacgg

SEQ ID NO: 3 - DNA, 97 bp, Artificial Sequence
  Primer
  misc_feature (59)..(73): n is a, c, g, or t
aatgatacggcgaccaccgagatctacactctttccctacacgacgctcttccgatctnnnnnnnnnnnnnnntcttgtggaaaggacgaaacaccg

SEQ ID NO: 4 - DNA, 98 bp, Artificial Sequence
  Primer
  misc_feature (25)..(30): n is a, c, g, or t
  misc_feature (65)..(73): n is a, c, g, or t
caagcagaagacggcatacgagatnnnnnngtgactggagttcagacgtgtgctcttccgatctnnnnnnnnntctactattctttcccctgcactgt

SEQ ID NO: 5 - DNA, 24 bp, Artificial Sequence
  5′ Adapter
tcttgtggaaaggacgaaacaccg

SEQ ID NO: 6 - DNA, 28 bp, Artificial Sequence
  Primer
ctcttgtagatctgttctctaaacgaac

SEQ ID NO: 7 - DNA, 20 bp, Artificial Sequence
  Primer
ggtccaccaaacgtaatgcg

SEQ ID NO: 8 - DNA, 20 bp, Artificial Sequence
  Primer
gcctggaccacaagtttgac

SEQ ID NO: 9 - DNA, 20 bp, Artificial Sequence
  Primer
tgaaattctgggagcatgac